(19) United States
(12) Patent Application Publication
(10) Pub. No.: US 2015/0057275 A1
(43) Pub. Date: Feb. 26, 2015

(54) METHODS AND MATERIALS FOR GENETIC ANALYSIS OF TUMORS

(71) Applicant: THE GENERAL HOSPITAL CORPORATION, BOSTON, MA (US)

(72) Inventors: Dora Dias-Santagata, Jamaica Plain, MA (US); Anthony John Iafrate, Boston, MA (US)

(21) Appl. No.: 14/475,155

(22) Filed: Sep. 2, 2014

Related U.S. Application Data
(63) Continuation of application No. 12/799,415, filed on Apr. 23, 2010.
(60) Provisional application No. 61/172,342, filed on Apr. 24, 2009.

(51) Int. Cl. C12Q 1/68

(52) U.S. Cl. CPC C12Q 1/6886; C12Q 2600/106; C12Q 2600/156; C12Q 2600/16; C12Q 2600/118
USPC 514/234.5; 506/2; 514/266.4; 506/16

(57) ABSTRACT

This invention relates generally to methods and materials for rapid detection of mutations for tumor genotyping.

### CROSS REFERENCE TO RELATED APPLICATION

This application is a continuation of U.S. application Ser. No. 12/799,415, filed on Apr. 23, 2010, which claims priority from U.S. Provisional Application Ser. No. 61/172,342, filed on Apr. 24, 2009, which are incorporated herein by reference in their entirety.

### TECHNICAL FIELD

The invention relates to methods and materials for rapid detection of mutations for tumor genotyping.

### BACKGROUND

The clinical management of cancer patients has traditionally relied on chemotherapeutic choices that are mostly dictated by pathologic tumor histology and organ of origin. In recent years, major efforts to define the molecular causes of cancer have revealed a wide number of genetic aberrations (Davies et al. (2005) Cancer Res 65, 7591-7595; Ding et al. (2008) Nature 455, 1069-1075; Greenman et al. (2007) Nature 446, 153-158; Rikova et al. (2007) Cell 131, 1190-1203; Sjoblom et al. (2006) Science 314, 268-274; Stephens et al. (2005) Nat Genet, 37 590-592; Thomas et al. (2007) Nat Genet 39, 347-351; Wood et al. (2007) Science 318, 1108-1113). A small subset of these defects, usually referred to as “drivers,” is frequently present across cancer types and appears to be essential for oncogenesis and tumor progression (Greenman et al. (2007) Nature 446, 153-158). A new generation of drugs has been developed to selectively target such cancer-promoting pathways (Druker et al. (2001) N Engl J Med 344, 1031-1037; Hanahan and Weinberg (2000) Cell, 100, 57-70; Weinstein, 2000) and hence, treatment dictated by genetic markers is starting to complement the more conventional therapeutic approaches.

### SUMMARY

The present invention is based, at least in part, on the discovery of a robust and highly sensitive tumor genotyping assay for real-time testing of tumors.

In one aspect, the invention features methods of providing a genetic profile of a tumor (e.g., a tumor cell from a lung, breast, colorectal, head and neck, or ovarian tumor, or any solid tumor or hematopoietic malignancy), the method comprising providing a sample comprising genomic DNA from a tumor cell and simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, thereby providing a genetic profile of the tumor.

In one embodiment, the methods described herein wherein the tumor cell is in a formalin-fixed paraffin-embedded biopsy sample.

In one embodiment, the methods described herein comprise determining the identity of about 6 to 9 alleles in a single reaction.

In one embodiment, the methods described herein comprise determining the identity of all alleles listed in Table 3B.

In one embodiment, the methods described herein comprise performing a plurality of reactions as set forth in Tables 8A and 8B.

In another aspect, the invention features methods of selecting an appropriate chemotherapy for a subject with cancer (e.g., lung cancer, breast cancer, colorectal cancer, head and neck cancer, ovarian cancer, any solid tumor or hematopoietic malignancy), the method comprising providing a sample comprising genomic DNA from a tumor cell from the subject; simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, to provide a genetic profile of the tumor; and selecting an appropriate chemotherapy based on the genetic profile of the tumor.

In one embodiment, if an EGFR 2369C>T, KRAS 34G>T, KRAS 34G>C, KRAS 34G>A, KRAS 35G>T, KRAS 35G>C, KRAS 35G>A, KRAS 37G>T, KRAS 37G>C, KRAS 37G>A, KRAS 38G>T, KRAS 38G>C, or KRAS 38G>A mutation is present, then a therapy comprising an EGFR inhibitor is not selected.

In one embodiment, the methods described herein comprise determining the identity of about 6 to 9 alleles in a single reaction.

In one embodiment, the methods described herein comprise determining the identity of all alleles listed in Table 3B.

In one embodiment, the methods described herein comprise performing a plurality of reactions as set forth in Tables 8A and 8B.

In one embodiment, the methods further comprise administering the selected chemotherapy (e.g., erlotinib or gefitinib) to the subject.

In one aspect, the invention features methods of determining a prognosis for a subject diagnosed with cancer (e.g., lung cancer, breast cancer, colorectal cancer, head and neck cancer, ovarian cancer, any solid tumor or hematopoietic malignancy), the method comprising providing a sample comprising genomic DNA from a tumor cell from the subject; simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, to provide a genetic profile of the tumor; and determining a prognosis for the subject based on the genetic profile of the tumor.

In one embodiment, the subject has a plurality of tumors and the method comprises determining the genetic profile of more than one tumor in the subject, wherein the presence of an identical profile in each tumor indicates that the cancer is metastatic (i.e., poor prognosis), and the presence of a different profile in each tumor indicates that the cancer is not metastatic (i.e., better prognosis). Further, a FTL3 2503G>T mutation indicates a poor prognosis in acute myeloid leukemia. All IDH1 mutations indicate better prognosis in glioblastoma.

In one embodiment, the methods described herein comprise determining the identity of about 6 to 9 alleles in a single reaction.

In one embodiment, the methods described herein comprise determining the identity of all alleles listed in Table 3B.

In one embodiment, the methods described herein comprise performing a plurality of reactions as set forth in Tables 8A and 8B.

In another aspect, the invention features kits comprising the primers listed in Table 7. In one embodiment, the primers are provided in a container in the combinations as listed in Tables 8A and 8B.

The term “single reaction” as used herein refers to a reaction occurring in a vessel, e.g., tube, well, area on an array, or other container, suitable for the purpose.

As used herein, an “allele” is one of a pair or series of genetic variants of a polymorphism at a specific genomic location. A “cancer susceptibility allele” is an allele that is associated with increased susceptibility of developing cancer.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

The term “chromosome” as used herein refers to a gene carrier of a cell that is derived from chromatin and comprises DNA and protein components (e.g., histones). The conventional internationally recognized individual human genome chromosome numbering identification system is employed herein.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figure, and from the claims.

### DESCRIPTION OF DRAWINGS

Tables 1 to 9 appear at the end of this text before the drawings.

The patent or application file contains at least one drawing executed in color. Copies of this patent or patent application publication with color drawings will be provided by the Office upon request and payment of the necessary fee.

FIG. 1A is a schematic representation of one embodiment of the present method of tumor genotyping. In this embodiment, the method consists of a multiplexed PCR step, followed by a single-base extension sequencing reaction, in which allele-specific probes interrogate loci of interest and are fluorescently labeled using dideoxynucleotides. These probes are designed to have different sizes and are subsequently resolved by electrophoresis and analyzed by an automated DNA sequencer. Thus, the identity of each locus is given by the position of its corresponding fluorescent peak in the spectrum, which is dictated by the length of the extension primer.

FIG. 1B is a detailed view of the single-base extension reaction. The identity of the nucleotide(s) present at each locus is given by two parameters: the molecular weight and the color of the fluorescently-labeled ddNTPs added to the allele specific probes during the extension step. Thus, mutant and wild-type alleles can be distinguished based on the slightly different positions and on the distinct colors of their corresponding peaks. These two factors are used to establish the bins used for automatic data analysis.

FIGS. 2A and 2B are each panels of five chromatograms from two representative assays. The section on the left represents the multiplexed panel containing the assay of interest; the middle section is a magnified image of the assay being tested and includes the bins used for automatic allele calling; and the section on the right represents traditional Sanger sequencing analysis of the same samples. In both cases, the top panel shows genotyping data obtained for normal male genomic DNA (SEQ ID NOS 237 and 239, respectively, in order of appearance) (Promega, Madison, Wis.). In the panels underneath, DNA derived from cancer cell lines harboring specific mutations (SEQ ID NOS 238 and 240, respectively, in order of appearance) was serially diluted against the wild-type genomic DNA (Promega), as specified by the percentage values on the left. Mutant alleles are indicated by arrows, and background signals are marked with asterisks. (A) The A427 lung carcinoma cell line was used to detect the KRAS G12D mutation (nucleotide change 35G>A). Sensitivity was ˜3% and the panel includes the following assays: (1) KRAS 35; (2) EGFR 2236_{—}50del R; (3) PTEN 517; (4) TP53 733; (5) FLT3 2503; (6) PIK3CA 3139; (7) NOTCH1 4724; and (8) NOTCH1 4802. (B) The NCI-H1975 lung adenocarcinoma cell line was used to identify the EGFR T790M mutation (nucleotide change 2369C>T). Assay sensitivity was ˜3% and the panel tests for: (1) KRAS 34; (2) EGFR 2235_{—}49del F; (3) EGFR 2369; (4) NRAS 181; (5) PIK3CA 1633; (6) CTNNB1 94; and (7) CTNNB1 121. As can be appreciated in the middle section, decreasing levels of “green” mutant signal (arrows), absent from wild-type DNA (top panel), can be easily distinguished from the nearby “red” background peak (asterisk), which is also found in the assay run on the normal control (top panel). Of note, the EGFR c.2369C assay was designed in the reverse orientation, thus the observed alleles are G (blue) for the wild-type and A (green) for the mutant. An in-depth view of sensitivity assessment for these two assays is illustrated in FIG. 7.

FIGS. 3A and 3B are two bar graphs showing the distribution of somatic mutations in primary human cancers. Mutational profiling of 250 cancer specimens is depicted across tumor types according to: (A) their mutational status and (B) the mutation frequency of individual genes.

FIGS. 4A-C are each three chromatogram profiles of primary tumors and matching normal tissue demonstrating assay specificity. Shown here are three examples of genotyping data obtained using total nucleic acid extracted from normal (top) (SEQ ID NOs:241, 243, and 245, respectively, in order of appearance) and tumor (middle) (SEQ ID NOs:242, 244, and 246, respectively, in order of appearance) FFPE tissue from the same individual, and a no-DNA negative control (bottom). Of note, the mutant allele (arrow) is only found in the tumor (middle panel). (A) Detection of the EGFR L858R (c.2573T>G) mutation in a case of lung adenocarcinoma. Assays: (1) EGFR 2236_{—}50del F; (2) EGFR 2573; (3) CTNNB1 133; (4) PIK3CA 1624; and (5) NRAS 35. (B) Identification of the KRAS G12V (c.35G>T) mutation in a pancreatic adenocarcinoma. Assays: (1) KRAS 35; (2) EGFR 2236_{—}50del R; (3) PTEN 517; (4) TP53 733; (5) FLT3 2503; (6) PIK3CA 3139; (7) NOTCH1 4724; and (8) NOTCH1 4802. (C) Detection of the BRAF V600E (c.1799T>A) mutation in melanoma. Assays: (1) EGFR 2235_{—}49del R; (2) NRAS 38; (3) BRAF 1799; (4) NRAS 182; (5) PIK3CA 263; (6) TP53 742; (7) CTNNB1 95; and (8) CTNNB1 122.

FIGS. 5A and 5B are each eight chromatograms showing representative spectra of the 58 SNAPSHOT® assays from (A) 20 ng of commercially available high-quality genomic DNA (Promega) and (B) 60 ng of total nucleic acid extracted from FFPE primary tumor tissue. Assays: I. (1) KRAS 34; (2) EGFR 2235_{—}49del F; (3) EGFR 2369; (4) NRAS 181; (5) PIK3CA 1633; (6) CTNNB1 94; and (7) CTNNB1 121. II. (1) EGFR 2235_{—}49del R; (2) NRAS 38; (3) BRAF 1799; (4) NRAS 182; (5) PIK3CA 263; (6) TP53 742; (7) CTNNB1 95; and (8) CTNNB1 122. III. (1) EGFR 2236_{—}50del F; (2) EGFR 2573; (3) CTNNB1 133; (4) PIK3CA 1624; and (5) NRAS 35. IV. (1) KRAS 35; (2) EGFR 2236_{—}50del R; (3) PTEN 517; (4) TP53 733; (5) FLT3 2503; (6) PIK3CA 3139; (7) NOTCH1 4724; and (8) NOTCH1 4802. V. (1) CTNNB1 110; (2) KRAS 38; (3) CTNNB1 134; (4) TP53 743; (5) TP53 817; and (6) APC 4666_{—}67insA. VI. (1) CTNNB1 98; (2) KRAS 37; (3) EGFR 2155; (4) KIT 2447; (5) PIK3CA 3145; (6) PIK3CA 1637; (7) APC 4012; and (8) TP53 818. VII. (1) PIK3CA 3140; (2) CTNNB1 101; (3) JAK2 1849; (4) BRAF 1798; (5) NRAS 37; (6) PIK3CA 1636; (7) APC 4348; and (8) APC 3340. VIII. (1) NRAS 34; (2) PTEN 388; (3) CTNNB1 109; (4) PTEN 697; (5) PTEN 800delA; (6) NRAS 183; (7) TP53 524; and (8) TP53 916.

FIGS. 6A and 6B are a table (A) and a bar graph (B) showing the sensitivity of the assay, which is on average 4.64%. A few examples of assay sensitivity are presented in FIGS. 2 and 8. A detailed illustration of data collection and the calculations involved in sensitivity assessment can be found in FIG. 7.

FIGS. 7A and 7B show chromatograms and tables of the sensitivity assessment illustrated in FIG. 2. The section on the left represents the assay being tested, with the sizes of wild-type and mutant alleles indicated on the left (f.u.=fluorescence units). Arrows in the high-power images in the middle section point to the background noise within the mutant bin in the genomic DNA sample (top) and to the mutant allele in the 3% dilution of the mutant sample (bottom). The top table depicts the levels of genomic (wild-type) and cell line (mutant) DNA within each sample, and the percentage of mutant allele obtained for each assay, calculated as a ratio of fluorescent peak heights [mutant*100/(wild type+mutant)]. The bottom table illustrates the calculations that selected the sample used to determine the sensitivity. Sensitivity of an assay was established as the lowest percentage of mutation in the test sample (arrow at the top table) yielding a mutant allele peak that was >3 times the background noise in the wild type sample (arrow at the bottom table). (A) The sensitivity of the KRAS G12D (c.35G>A) assay is 3.0%, which was determined using the sample with 3% of A427 cell line DNA. (B) The sensitivity of the EGFR T790M (c.2369C>T) SNAPSHOT® assay is 3.2%, which was established using the sample containing 3% of NCI-H1975 cell line DNA.

FIG. 8 is a series of chromatograms showing sensitivity testing using cancer cell line DNA. The NCI-H1975 lung adenocarcinoma cell line was used to identify the EGFR L858R (c.2573T>G) mutation. Sensitivity was ˜5%. Assays: (1) EGFR 2236_{—}50del F; (2) EGFR 2573; (3) CTNNB1 133; (4) PIK3CA 1624; and (5) NRAS 35. FIG. 8 discloses “Genomic DNA” as SEQ ID NO:247 and “NCI-H1975 100%” as SEQ ID NO:248.

FIGS. 9A and 9B are each three chromatograms validating the assay using synthetic oligonucleotides. Synthetic DNA primers designed to harbor specific mutations (Table 10) were used to validate the assays for absent primary tumor or cell line controls. Both cases illustrate the genotyping results obtained using wild-type genomic DNA (Promega) (top), 3 pmol of synthetic oligonucleotide added to wild-type genomic DNA (middle), and a no-DNA control (bottom). (A) The A.ctrl_CTNNB1_{—}110C>G control primer was used to identify the CTNNB1S37C (c.110C>G) mutation. Assays: (1) CTNNB1 110; (2) KRAS 38; (3) CTNNB1 134; (4) TP53 743; (5) TP53 817; and (6) APC 4666_{—}67insA. (B) The A.ctrl_PTEN_{—}388C>T control primer was used to detect the PTENR130X (c.388C>T) mutation. Assays: (1) NRAS 34; (2) PTEN 388; (3) CTNNB1 109; (4) PTEN 697; (5) PTEN 800delA; (6) NRAS 183; (7) TP53 524; and (8) TP53 916.

FIGS. 10A and 10B are each a series of chromatograms illustrating examples of rare mutations detected by SNAPSHOT® genotyping. (A) Co-occurrence of the KRASG12V (c.35G>T) (upper) and PIK3CAE545K (1633G>A) (lower) mutations in a case of breast lobular carcinoma. Both images show genotyping data obtained using total nucleic acid extracted from normal (top) and tumor (middle) FFPE tissue from the same individual, and a no-DNA negative control (bottom). Upper image assays: (1) KRAS 35; (2) EGFR 2236_{—}50del R; (3) PTEN 517; (4) TP53 733; (5) FLT3 2503; (6) PIK3CA 3139; (7) NOTCH1 4724; and (8) NOTCH1 4802. Lower image assays: (1) KRAS 34; (2) EGFR 2235_{—}49del F; (3) EGFR 2369; (4) NRAS 181; (5) PIK3CA 1633; (6) CTNNB1 94; and (7) CTNNB1 121. (B) Co-occurrence of the CTNNB1S37F (c.110C>T) (upper) and EGFRE746_A750del (c.2235_{—}2249del15) (lower) mutations in a case of fetal lung adenocarcinoma. Both images show the results obtained using wild type genomic DNA (Promega) (top), total nucleic acid extracted from FFPE primary tumor tissue (middle), and a no-DNA negative control (bottom). Upper image assays: (1) CTNNB1 110; (2) KRAS 38; (3) CTNNB1 134; (4) TP53 743; (5) TP53 817; and (6) APC 4666_{—}67insA. Lower image assays: (1) KRAS 34; (2) EGFR 2235_{—}49del F; (3) EGFR 2369; (4) NRAS 181; (5) PIK3CA 1633; (6) CTNNB1 94; and (7) CTNNB1 121.

FIGS. 11A and 11B are a series of two tables and a bar graph showing classes of mutations found in primary tumors (A) across tumor types and (B) correlation with smoking history.

FIGS. 12A-C are panels of chromatograms showing that targeted mutational profiling impacts clinical management. Genomic DNA or total nucleic acid extracted from normal (top) and tumor (middle) FFPE tissue from the same patient was run in parallel with a no-DNA negative control (bottom). (A) Identification of the PIK3CAH1047L (c.3140A>T) mutation in breast cancer. Of note, the PIK3CA c.3140A assay was designed in the reverse orientation, thus the observed alleles are T (red) for the wild-type and A (green) for the mutant. Assays: (1) PIK3CA 3140; (2) CTNNB1 101; (3) JAK2 1849; (4) BRAF 1798; (5) NRAS 37; (6) PIK3CA 1636; (7) APC 4348; and (8) APC 3340. (B) Detection of three mutations in a case of lung adenocarcinoma: EGFRE746_A750del (c.2235_{—}2249del15) and EGFRT790M (c.2369C>T) (upper) and TP53R175H (c.524G>A) (lower). Upper image assays: (1) KRAS 34; (2) EGFR 2235_{—}49del F; (3) EGFR 2369; (4) NRAS 181; (5) PIK3CA 1633; (6) CTNNB1 94; and (7) CTNNB1 121. Lower image assays: (1) NRAS 34; (2) PTEN 388; (3) CTNNB1 109; (4) PTEN 697; (5) PTEN 800delA; (6) NRAS 183; (7) TP53 524; and (8) TP53 916. (C) Distinct genotypes found in two tumor masses resected from a lung adenocarcinoma patient. Identification of the KRASG12C (c.34G>T) mutation in the right lung resection (upper), and the KRASG12A (c.35G>C) mutation in the left lung resection (lower). Of note, the proportion of tumor vs. normal cells was different in the two specimens (75% of tumor in the right lung resection and 30-40% of tumor in the left lung resection), which partly explains the distinct mutant vs. wild-type allele ratios observed in the two samples. Upper image assays: (1) KRAS 34; (2) EGFR 2235_{—}49del F; (3) EGFR 2369; (4) NRAS 181; (5) PIK3CA 1633; (6) CTNNB1 94; and (7) CTNNB1 121. Lower image assays: (1) KRAS 35; (2) EGFR 2236_{—}50del R; (3) PTEN 517; (4) TP53 733; (5) FLT3 2503; (6) PIK3CA 3139; (7) NOTCH1 4724; and (8) NOTCH1 4802.

FIGS. 13A and 13B are a series of chromatograms comparing the present methods and Sequenom MassARRAY genotyping methods. Wild-type genomic DNA (top) and total nucleic acid extracted from an FFPE lung adenocarcinoma specimen harboring the KRAS G12D mutation (bottom) were analyzed using SNAPSHOT® and Sequenom MassARRAY. The arrow marks the mutant allele. Three assays are depicted for each method. (A) SNAPSHOT® platform: automatic allele calling is based on a pre-established binning system that incorporates two sources of information: molecular weight (of the extension product) and color (of the fluorescently-labeled dideoxynocleotide that is added onto each extension probe during the single base extension reaction). Assays: (1) KRAS 35; (2) EGFR 2236_{—}50del R; and (3) PTEN 517. (B) Sequenom MassARRAY method: allele calling is based on the distinct molecular weights of each extension product. In addition to the wild-type (w) and three potential mutant (m) signals, the spectral output of each Sequenom MassARRAY assay will also include a peak corresponding to the remaining unextended primer (u). Assays: (1) KRAS 35; (2) EGFR 2235_{—}2249del R; and (3) EGFR 2236_{—}50del F. The baseline background noise for the Sequenom MassARRAY was higher than with SNAPSHOT®. Of note, to test one sample with the SNAPSHOT® assay presented in this study, eight multiplexed panels, one chemistry, and one extension reaction mix were used. The protocol designed by Sequenom scientists to test the same loci included: 14 multiplexed panels, two chemistries (IPLEX and hME), and four distinct extension reaction mixes, which would have been more labor intensive, more expensive, and would require ˜75% more tumor tissue than the methods described herein.

FIGS. 14A to 14AG show the coding sequences (nucleic acid and corresponding amino acid) for AKT1, APC, BRAF, CTNNB1, EGFR, FLT3, IDH1, JAK2, KIT, KRAS, MAP2K1, NOTCH1, NRAS, PIK3CA, PTEN, and TP53.

### DETAILED DESCRIPTION

The methods and materials described herein are based, at least in part, on the development of a robust and highly sensitive tumor genotyping assay for real-time testing of tumors, which can assist physicians in directing their cancer patients to the most appropriate targeted therapies.

While the clinical benefit observed with some targeted agents is encouraging, it is clear that for such strategies to be successful, it is necessary to identify the patient population carrying the genetic abnormalities targeted by each drug (McDermott et al. (2007) Proc Natl Acad Sci USA 104, 19936-19941; Sos et al. (2009) J Clin Invest 119, 1727-1740). In advanced non-small cell lung cancer (NSCLC), activating mutations in the region encoding the kinase domain of the epidermal growth factor receptor (EGFR) gene predict tumor sensitivity to the tyrosine kinase inhibitors (TKI) erlotinib and gefitinib (Lynch et al. (2004) N Engl J Med 350, 2129-2139; Paez et al. (2004) Science 304, 1497-1500; Pao et al. (2004) Proc Natl Acad Sci USA 101, 13306-13311; Sordella et al. (2004) Science 305, 1163-1167). Since NSCLC patients harboring EGFR mutations benefit from these specific inhibitors in the first-line setting compared to standard chemotherapy (Mok et al. (2009) N Engl J Med 361, 947-957), and only a small fraction of NSCLCs harbor these mutations, prospective screening for EGFR mutations at the time of diagnosis is becoming common practice (Sharma et al. (2007) Nat Rev Cancer 7, 169-181). Equally important is the identification of mutations that render tumors resistant to therapy. Activating mutations in KRAS predict resistance to EGFR TKI treatment in NSCLC (Pao et al. (2005b) PLoS Med 2, e17). In metastatic colorectal cancer, mutations in KRAS, BRAF, and PIK3CA are associated with resistance to treatment with monoclonal antibodies cetuximab and panitumumab, which target the extracellular domain of EGFR (Di Nicolantonio et al. (2008) J Clin Oncol 26, 5705-5712; Lievre et al. (2006) Cancer Res 66, 3992-3995; Sartore-Bianchi et al. (2009) Cancer Res, 69, 1851-1857). Similarly in breast cancer, oncogenic mutations in PIK3CA or low levels of PTEN expression may confer resistance to treatment with trastuzumab, a monoclonal antibody that targets the HER2/NEU receptor (Berns et al. (2007) Cancer Cell 12, 395-402).

Pharmacogenomics is the branch of pharmacology that deals with the influence of genetic variation on drug response in patients by correlating gene expression or single-nucleotide polymorphisms with a drug's efficacy or toxicity. As the repertoire of selective therapeutic compounds continues to expand, the need to evaluate larger numbers of genetic mutations is a major challenge (Chin and Gray (2008) Nature 452, 553-563). Pharmacogenomics aims to develop rational means to optimize drug therapy, with respect to a subject's genotype, to ensure maximum efficacy with minimal adverse effects. Such approaches promise the advent of “personalized medicine,” in which drugs and drug combinations are optimized for an individual's unique genetic makeup.

In addition to the dilemma of selecting the most relevant abnormalities, the tissue samples themselves pose many obstacles, including minute specimens derived from small core biopsies, poor quality fragmented nucleic acid due to formalin fixation and paraffin embedding (FFPE) required for histology-based diagnosis (Srinivasan et al. (2002) Am J Pathol 161, 1961-1971), and heterogeneous tumor samples comprised of normal tissue and cancerous cells which dilute the mutant alleles of interest. Thus, a clinical assay should: (1) be multiplexed, to maximize information retrieval from limited tissue; (2) perform well with FFPE-derived material; and (3) be very sensitive to detect low-level mutations. Additionally, the turn-around-time for the entire specimen processing and mutation detection platform should be fast, in order to integrate into the rapid pace of clinical decision making and impact patient management.

Provided herein are methods for providing a genetic profile of a tumor. The present disclosure also describes predictive biomarkers (SNP alleles) to classify a tumor, e.g., as resistant or sensitive to a chemotherapeutic drug. The tumor can be from a subject, e.g., a human or animal, such as laboratory animals, e.g., mice, rats, rabbits, or monkeys, or domesticated and farm animals, e.g., cats, dogs, goats, sheep, pigs, cows, horses, and birds.

The biomarkers and methods are also useful in selecting appropriate therapeutic modalities for subjects with certain conditions, e.g., cancer, e.g., lung cancer, breast cancer, colon cancer, pancreatic cancer, renal cancer, stomach cancer, liver cancer, bone cancer, leukemia, lymphoma, multiple myeloma, hematological cancer, neural tissue cancer, melanoma, thyroid cancer, ovarian cancer, testicular cancer, prostate cancer, cervical cancer, vaginal cancer, or bladder cancer. A subject with cancer can be identified using methods known in the art, e.g., based on detection of a tumor or neoplasm, or on the presence of one or more symptoms of the condition. Symptoms of cancer vary greatly and are well-known to those of skill in the art and include, without limitation, breast lumps, nipple changes, breast cysts, breast pain, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, or pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, pancreas metastases, difficulty swallowing, and the like.

Furthermore, after performing any of the methods for characterizing the drug sensitivity of a tumor, the tumor can be subjected to any of a variety of chemotherapeutic drugs, e.g., any of those described above. It is understood that such therapies would be administered to a tumor that had been found by such a method to have an increased sensitivity to the therapy.

### Single Nucleotide Polymorphisms and Sensitivity to Drug Therapy

A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “T” at the polymorphic site, the altered allele can contain a “C”, “G” or “A” at the polymorphic site.

A series of SNP alleles have been identified that are associated with cancers (Tables 3A and 3B). Thus, the presence of one or more of these SNP alleles can be used to provide a genetic profile of a tumor and characterize the drug sensitivity of the tumor. The SNP genotypes (identified by their SNP site) are depicted in Tables 3A and 3B. Further information on the SNPs can be obtained from, for example, the COSMIC/Sanger Institute database that is accessible via the Internet.

In some embodiments, the allele(s) of at least one (e.g., at least two, at least three, at least four, at least five, at least six, at least seven, at least eight, at least nine, at least 10, at least 11, at least 12, at least 13, at least 14, at least 15, at least 20, at least 30, at least 40, at least 50, at least 80, at least 100, at least 120, or at least 140) of the SNP sites depicted in Table 3B can be determined and/or used to characterize the drug sensitivity of the tumor.

Methods for detecting the presence of a SNP are known in the art and include, for example, those set forth in the accompanying Examples. The methods of detecting a SNP can be performed in formats that allow for rapid preparation, processing, and analysis of multiple samples (see below). The methods will be described primarily with SNAPSHOT®, although it will be understood by skilled practitioners that they may be adapted for use with other platforms, which may include standard Sanger sequencing, Sequenom MassARRAY, SNPStream and SNPlex technologies, among others. Further, a variety of reporter molecules can be used to determine the identity of an allele. For example, rather than fluorescent dideoxynucleotides, the single base extension reaction can be performed with oligonucleotides labeled with quantum dots; see, e.g., Sapsford et al. (2006) Sensors 6, 925-953. Alternatively, SNP detection can be performed by analysis of the molecular weight of the extension products using MALDI-TOFF mass spectrometry (Tang et al. (1999) Proc Natl Acad Sci USA 96:10016-20).

### Samples and Sample Collection

Suitable biological samples for the methods described herein include any biological fluid, cell, tissue, or fraction thereof, which includes analyte biomolecules of interest such as nucleic acid (e.g., DNA). A biological sample can be, for example, a specimen obtained from a human subject or can be derived from such a subject. For example, a sample can be a tissue section obtained by biopsy, or cells that are placed in or adapted to tissue culture. A biological sample can also be a biological fluid such as urine, blood, plasma, serum, saliva, semen, sputum, cerebral spinal fluid, tears, or mucus, or such a sample absorbed onto a paper or polymer substrate. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample.

The biological samples can be obtained from a subject, e.g., a subject having a tumor. Any suitable methods for obtaining the biological samples can be employed, although exemplary methods include, e.g., phlebotomy, swab (e.g., buccal swab), or fine needle aspirate biopsy procedure. Non-limiting examples of tissues susceptible to fine needle aspiration include lymph node, lung, thyroid, breast, and liver. Samples can also be collected, e.g., by microdissection (e.g., laser capture microdissection (LCM) or laser microdissection (LMD)), bladder wash, smear (PAP smear), or ductal lavage.

Methods for obtaining and/or storing samples that preserve the activity or integrity of molecules (e.g., nucleic acids) in the sample are well known to those skilled in the art. For example, a biological sample can be further contacted with one or more additional agents such as appropriate buffers and/or inhibitors, including nuclease inhibitors, which preserve or minimize changes in the molecules (e.g., nucleic acids) in the sample. Such inhibitors include, for example, chelators such as ethylenediamine tetraacetic acid (EDTA) and ethylene glycol bis(P-aminoethyl ether) N,N,N1,N1-tetraacetic acid (EGTA). Appropriate buffers and conditions for isolating molecules are well known to those skilled in the art and can be varied depending, for example, on the type of molecule in the sample to be characterized (see, for example, Ausubel et al., Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz, Textbook of Clinical Chemistry, 3rd ed. Burtis and Ashwood, eds. W.B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography.

For use in the methods described herein, a sample can be in a variety of physical states. For example, a sample can be a liquid or solid, can be dissolved or suspended in a liquid, can be in an emulsion or gel, and can be absorbed onto a material.

Subjects of all ages can be affected by cancer. Therefore, a biological sample used in a methods described herein can be obtained from a subject (e.g., a human) of any age, including a child, an adolescent, or an adult, such as an adult having a tumor.

### Applications

The methods and compositions described herein can be used to, e.g., (a) provide a genetic profile of a tumor and/or (b) characterize the drug sensitivity of a tumor. The profile can include information that indicates the presence or absence of one or more SNP genotypes depicted in Tables 3A and 3B.

The genetic profiles described herein can include information on the presence or absence of at least one or more (e.g., at least two or more, at least three or more, at least four or more, at least five or more, at least six or more, at least seven or more, at least eight or more, at least nine or more, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22, at least 24 or more, at least 30 or more, at least 40 or more, at least 50 or more, at least 80 or more, at least 100 or more, at least 120 or more, or at least 140 or more) SNP alleles depicted in Table 3B.

Grouping of multiple SNPs (e.g., 2, 3, 4, 5, 6, 7, 8, 9, 10, 15, 20, 25, 30, 35, 40, 45, 50, 70, 100, 120, or 140 or more SNPs depicted in Table 3B) into sets or clusters can improve the sensitivity or specificity of the method. A group of SNPs comprising individual SNPs selected from each of the clusters can then be tested for predictive accuracy and the classifiers can be recalculated based on the group of SNPs.

After profiling and characterizing the drug sensitivity of a tumor, a medical practitioner (e.g., a physician) can select an appropriate therapeutic modality for the subject (e.g., chemotherapeutic drugs selected from the group consisting of erlotinib, gefitinib, cetuximab, panitumumab, cisplatin, carboplatin, procarbazine, mechlorethamine, cyclophosphamide, camptothecin, adriamycin, ifosfamide, melphalan, chlorambucil, bisulfan, nitrosurea, dactinomycin, daunorubicin, doxorubicin, bleomycin, plicomycin, mitomycin, etoposide, verampil, podophyllotoxin, tamoxifen, taxol, transplatinum, 5-flurouracil, vincristin, vinblastin, methotrexate, and an analog of any of the aforementioned. Selecting a therapy for a subject can be, e.g.: (i) writing a prescription for a medicament; (ii) giving (but not necessarily administering) a medicament to a subject (e.g., handing a sample of a prescription medication to a patient while the patient is at the physician's office); (iii) communication (verbal, written (other than a prescription), or electronic (email, post to a secure site)) to the patient of the suggested or recommended therapeutic modality (e.g., non-immunosuppresive therapy or immunosuppresive therapy); or (iv) identifying a suitable therapeutic modality for a subject and disseminating the information to other medical personnel, e.g., by way of patient record. The latter (iv) can be useful in a case where, e.g., more than one therapeutic agent are to be administered to a patient by different medical practitioners.

It is understood that genetic profile of a tumor can be in electronic form (e.g., an electronic patient record stored on a computer or other electronic (computer-readable) media such as a DVD, CD, or floppy disk) or written form.

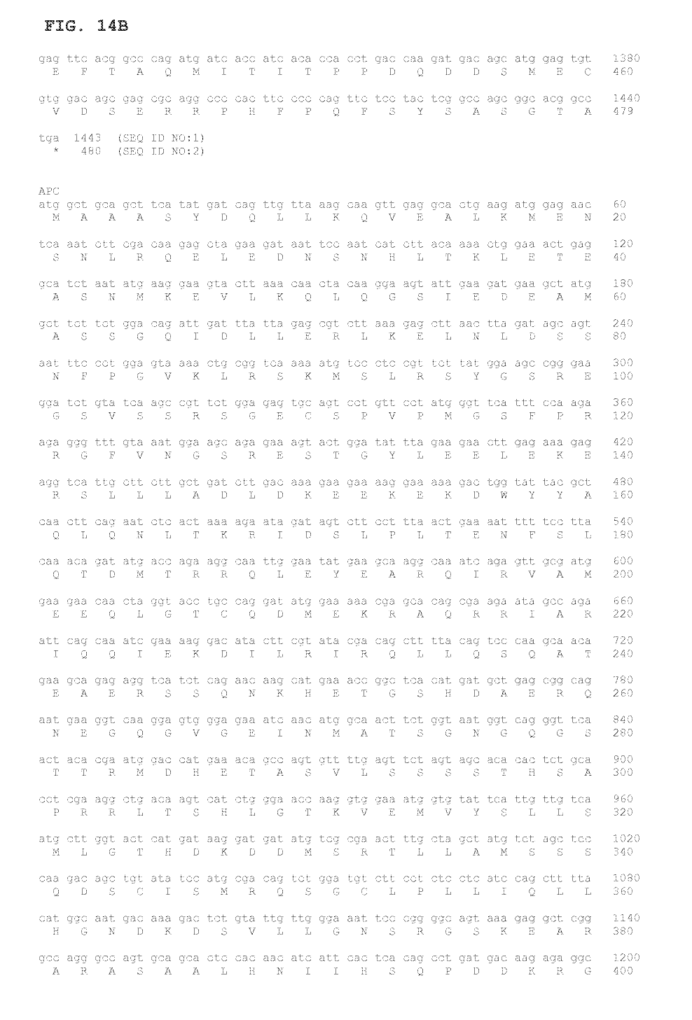

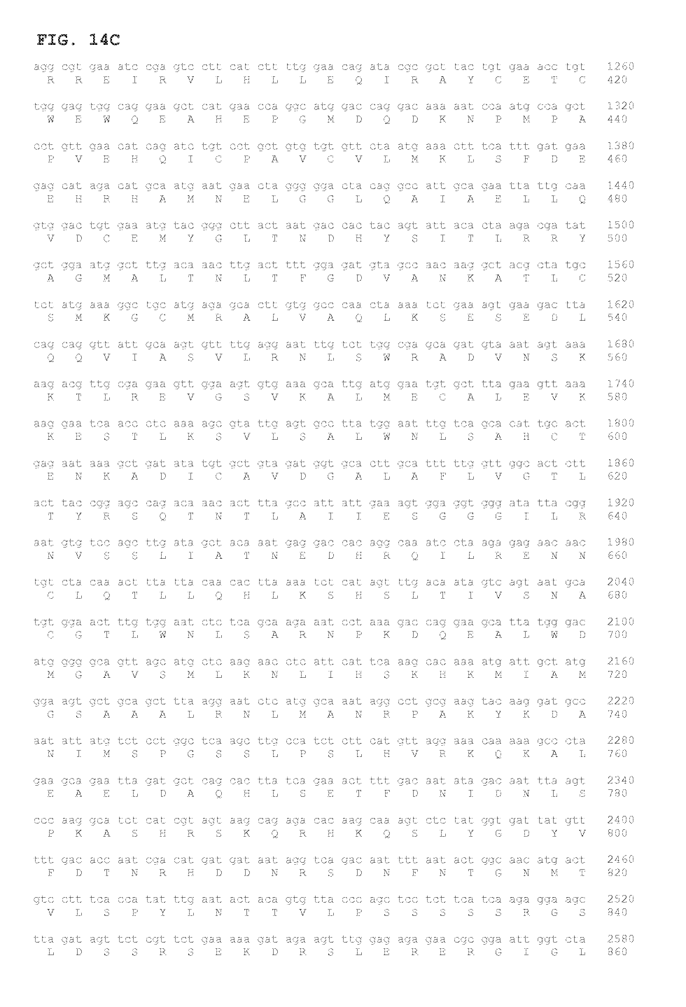

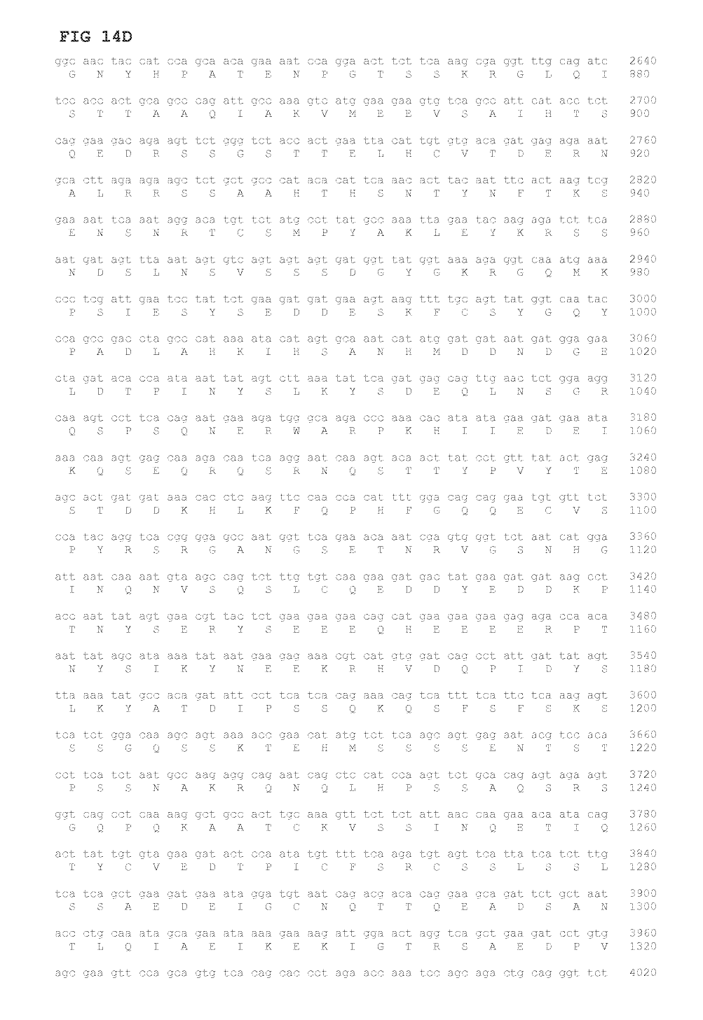

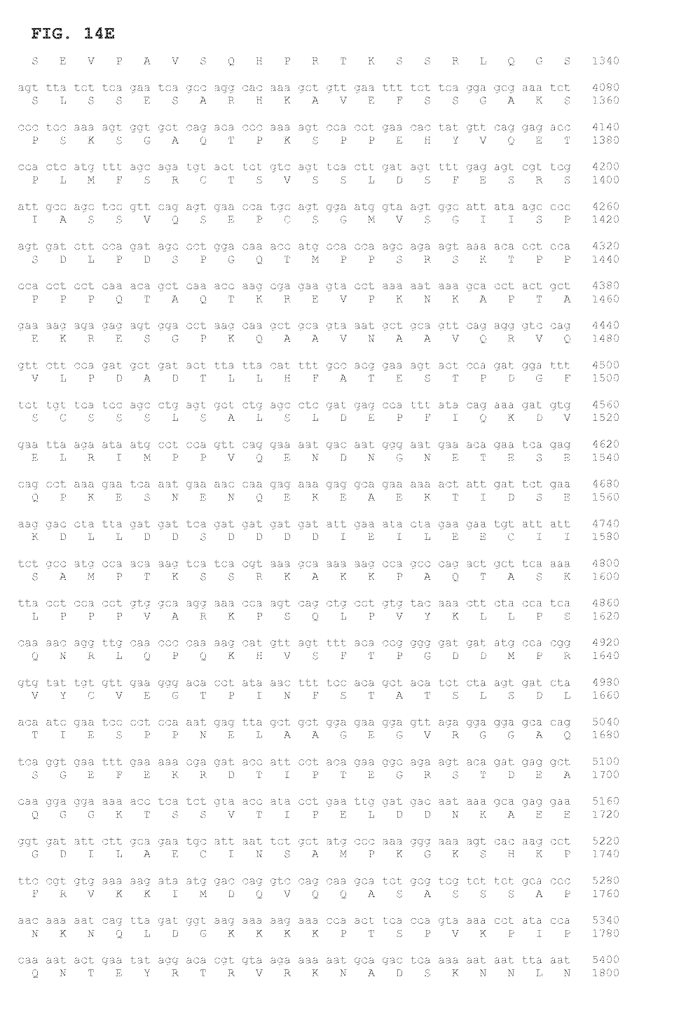

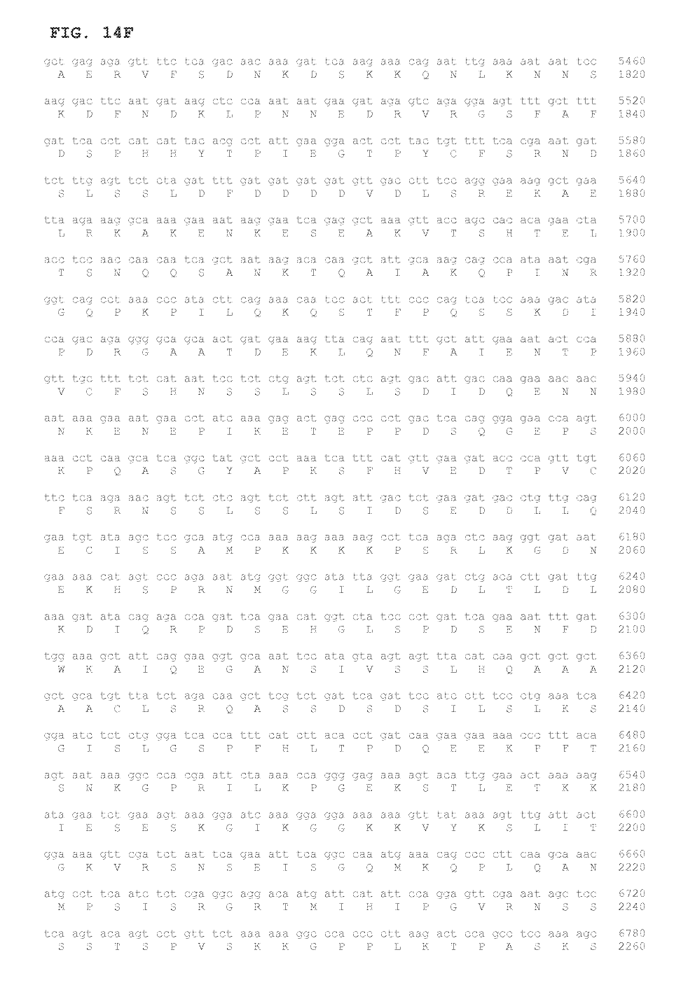

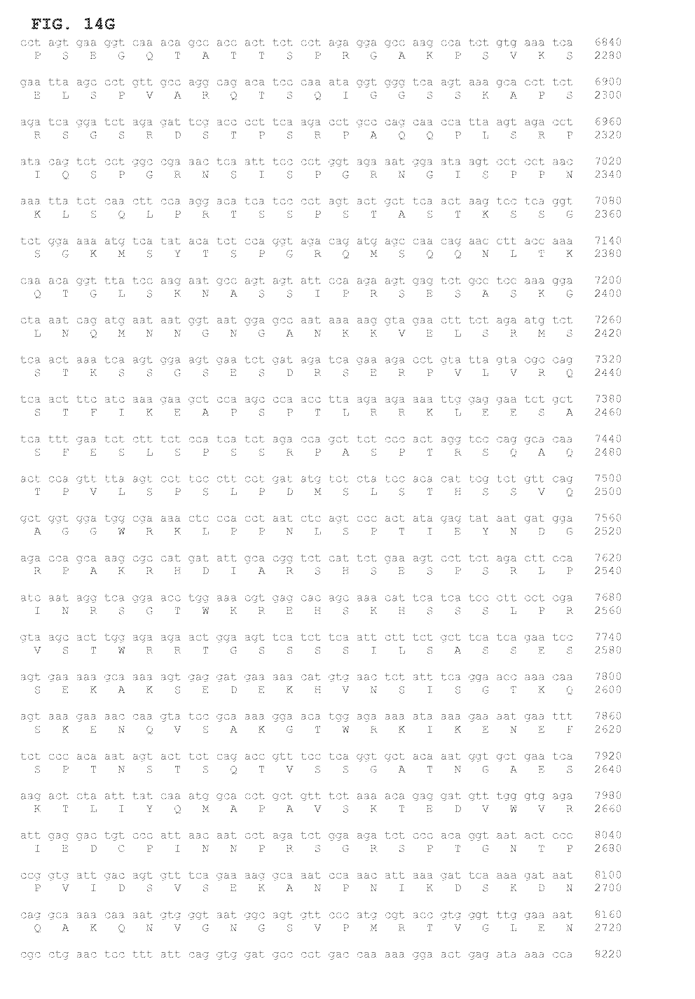

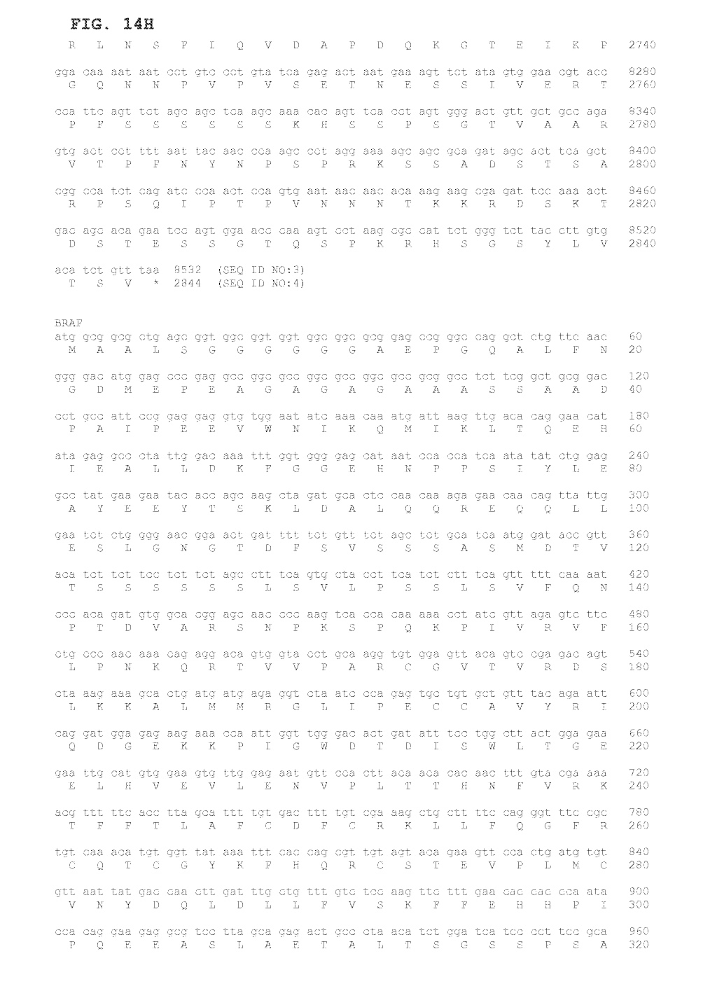

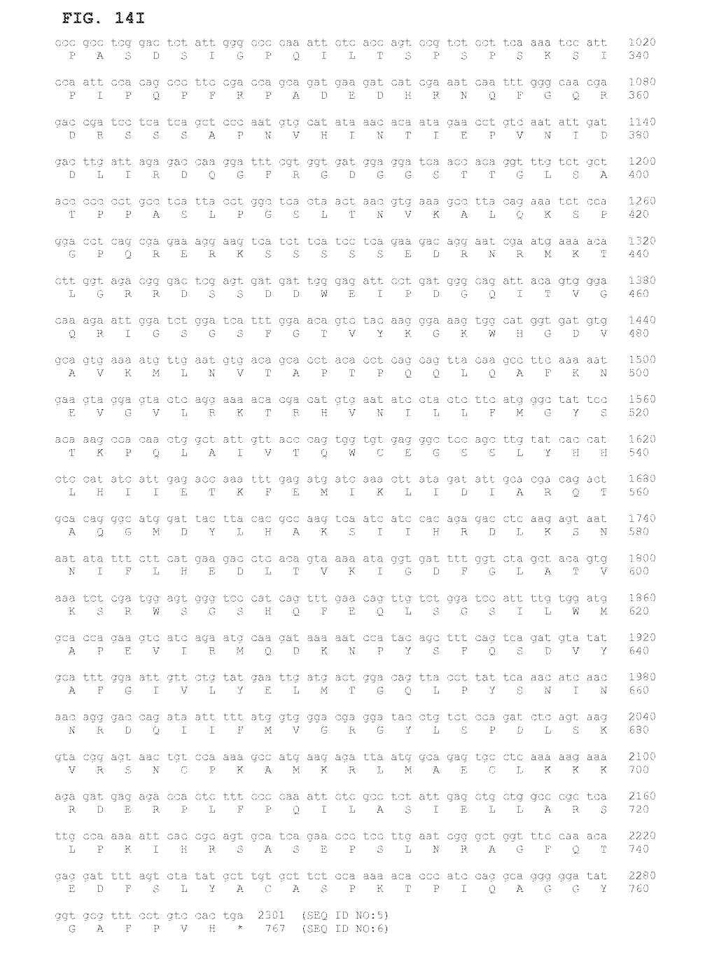

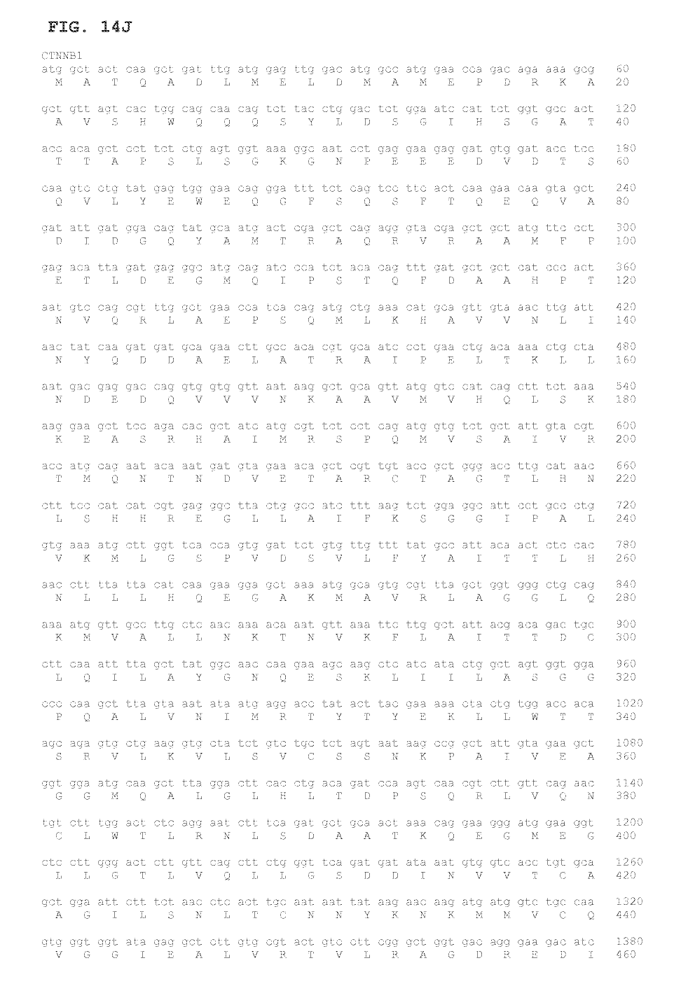

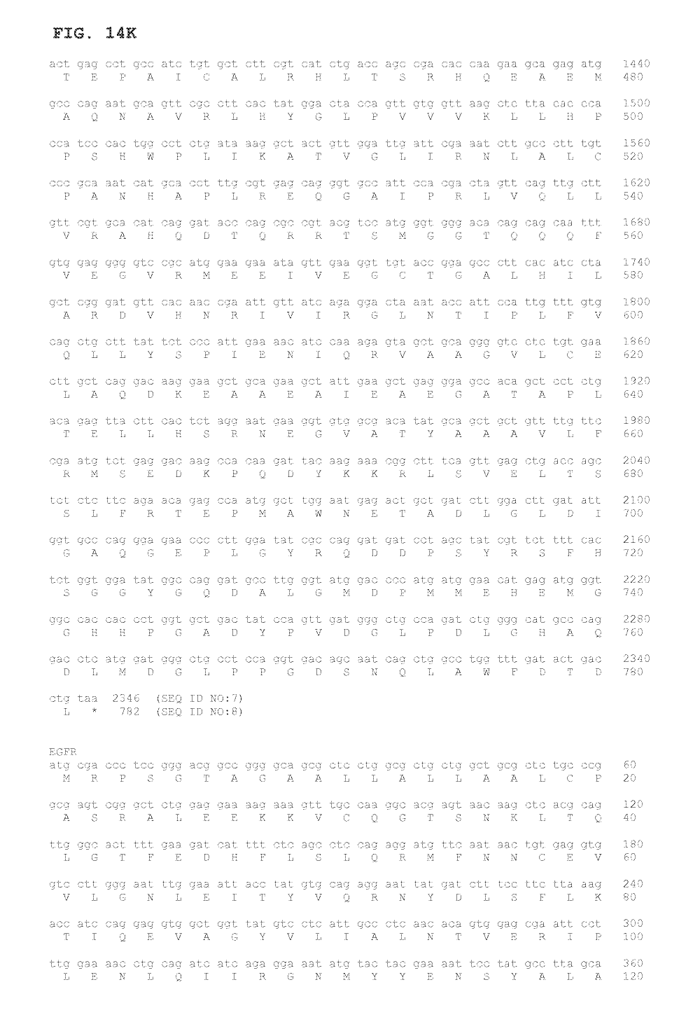

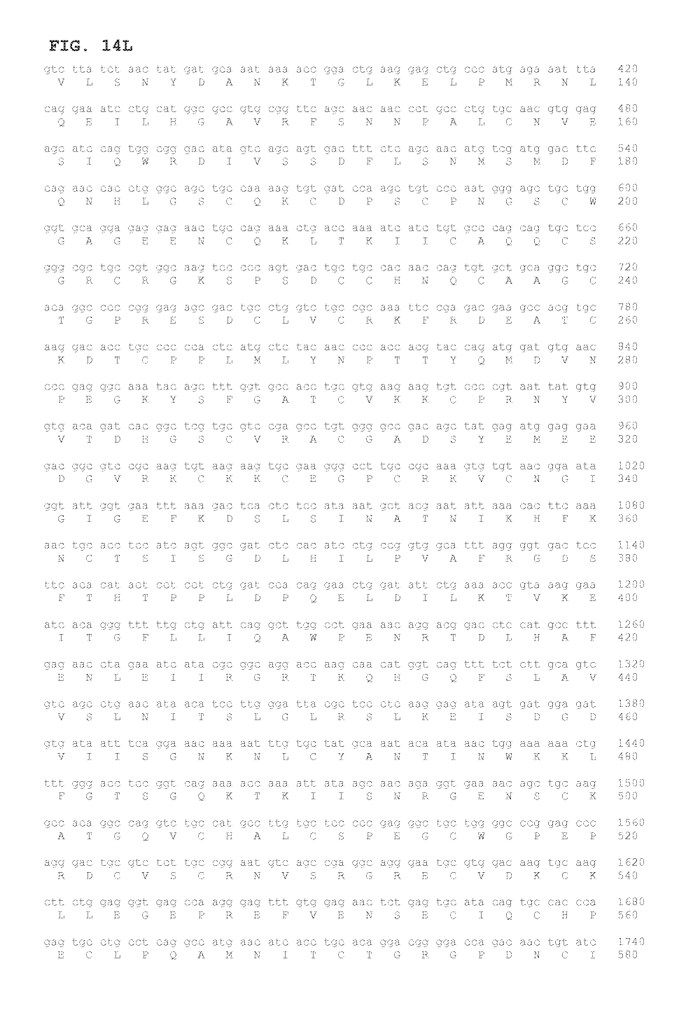

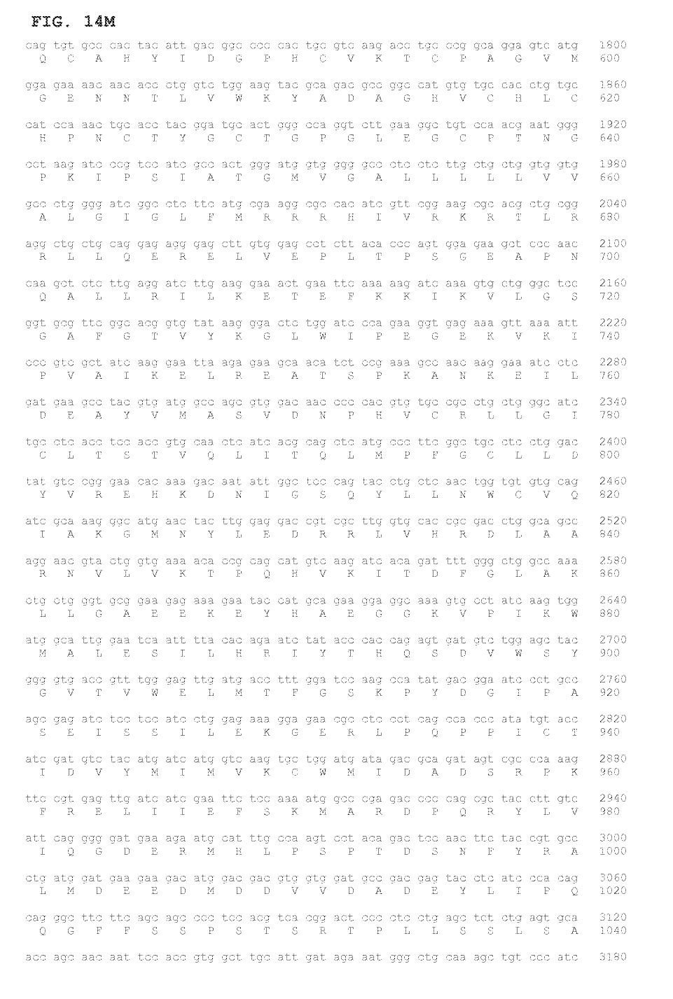

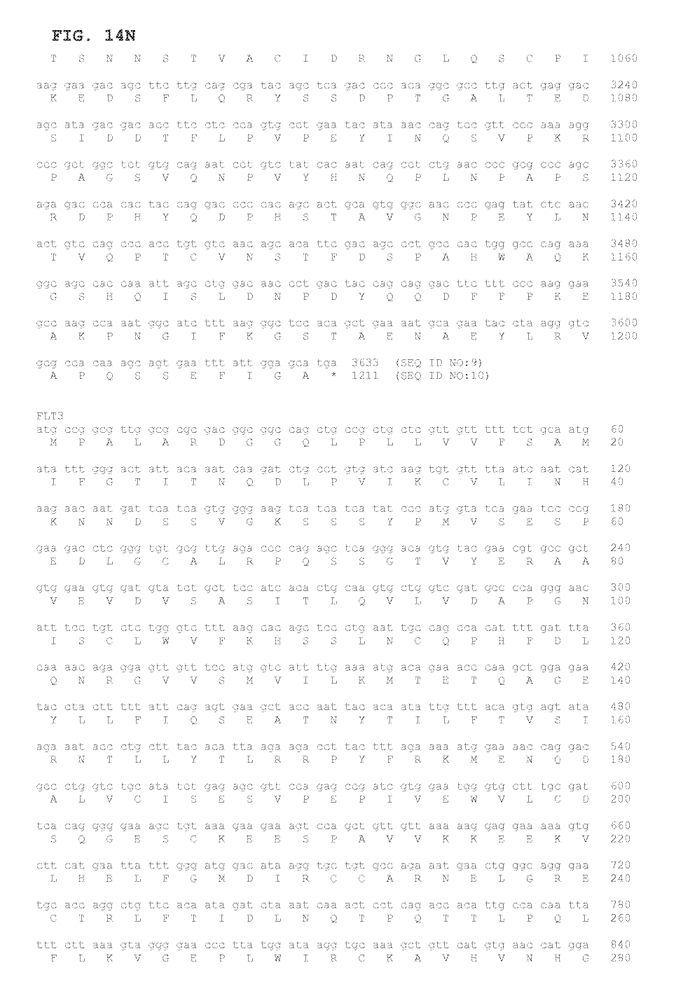

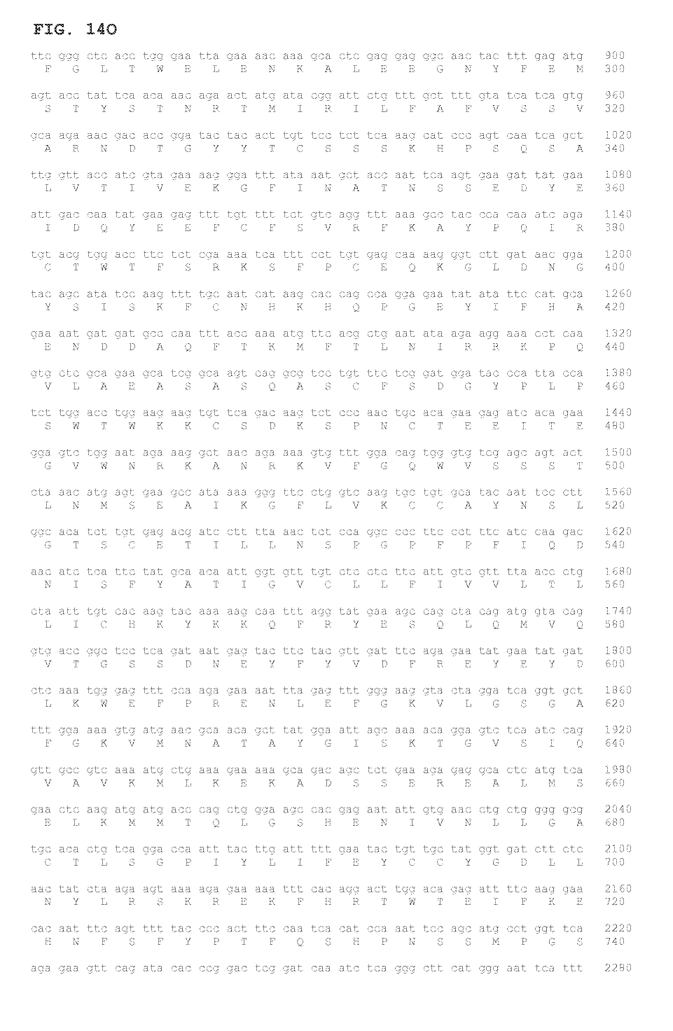

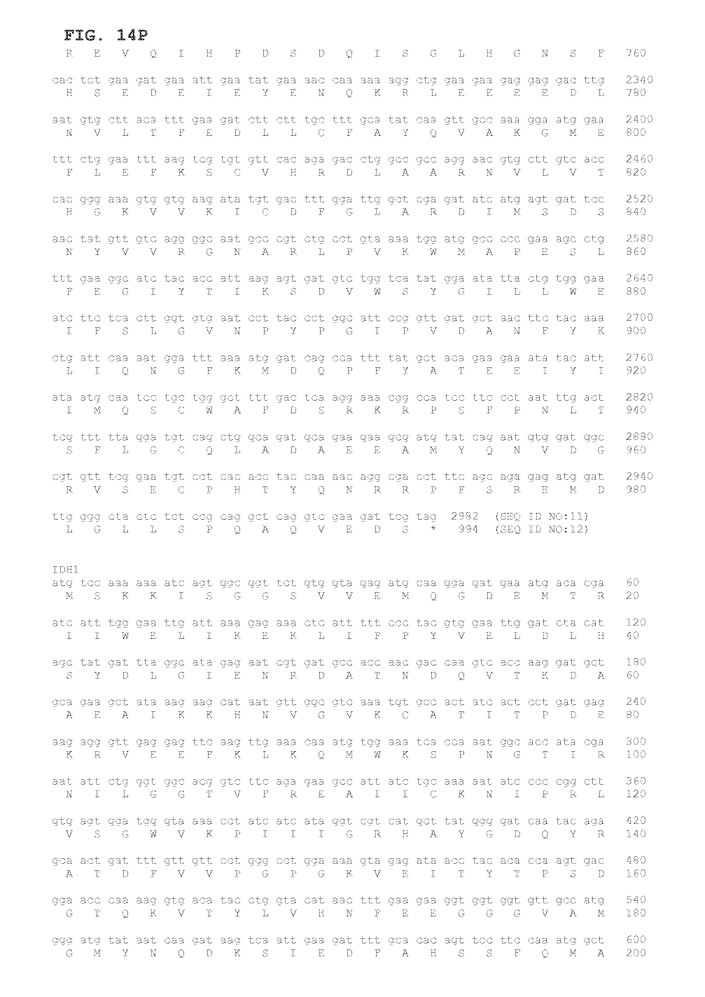

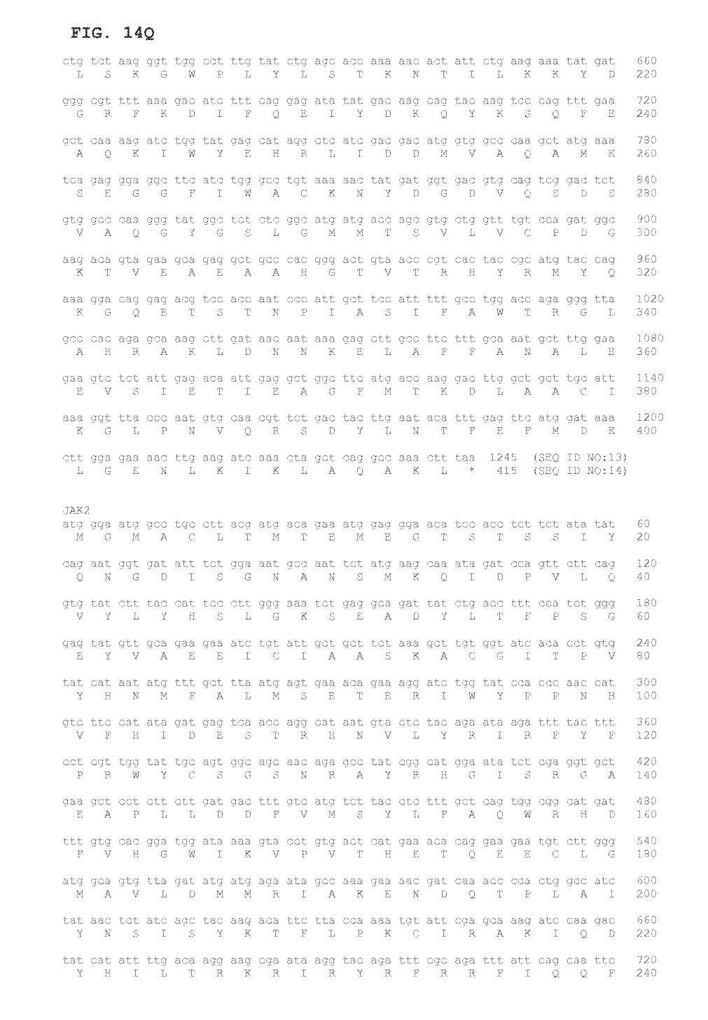

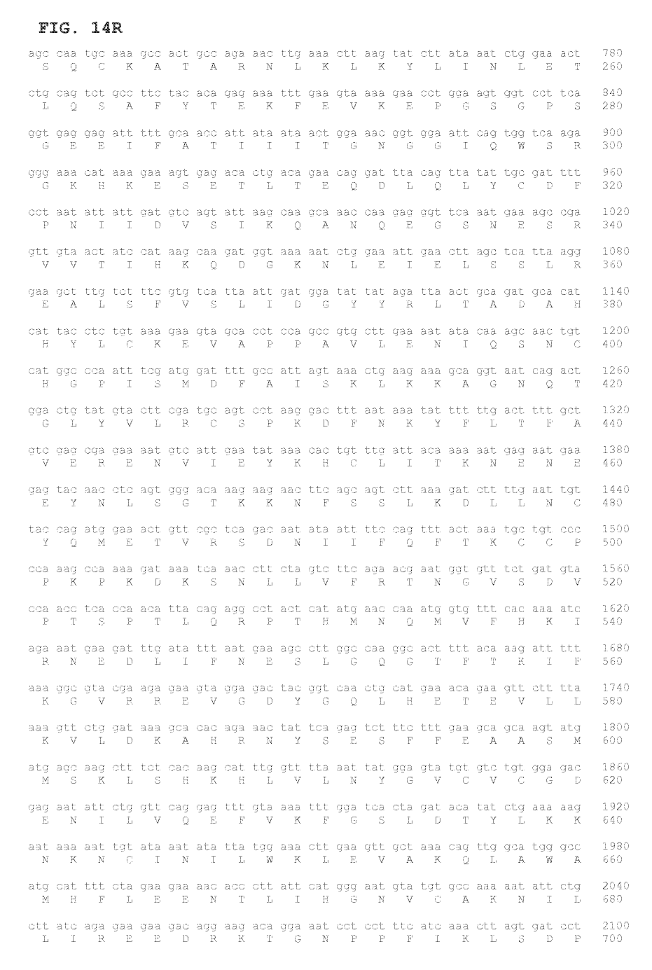

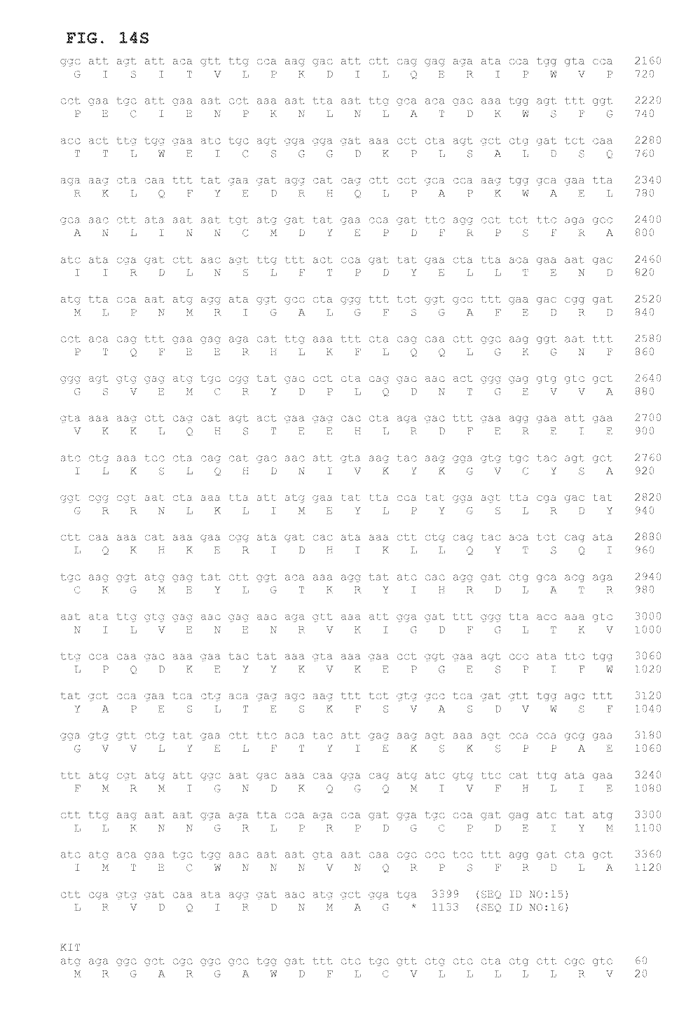

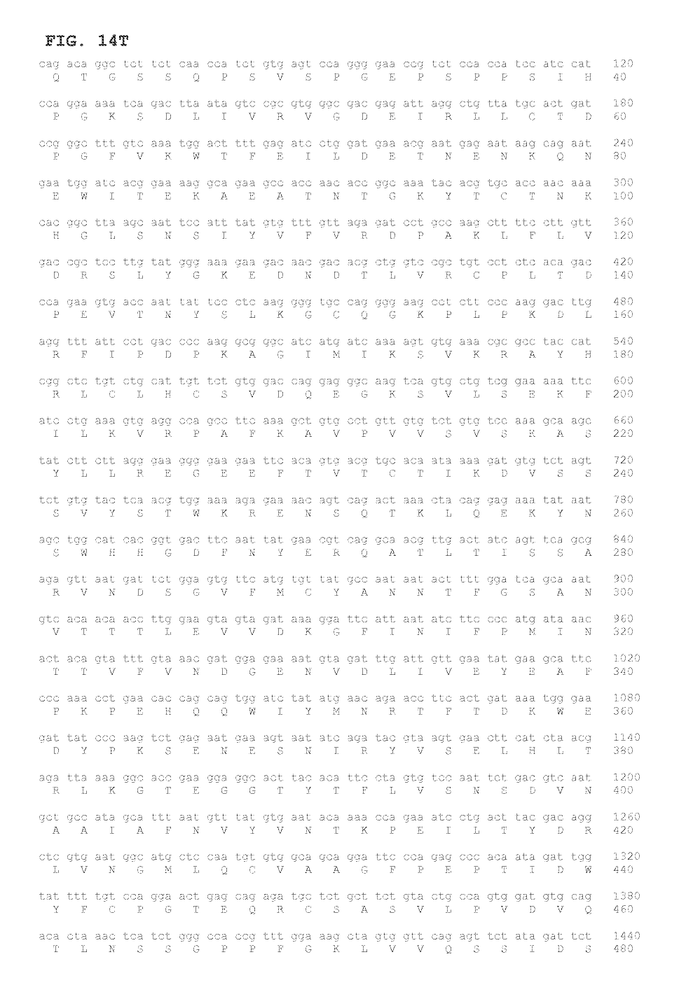

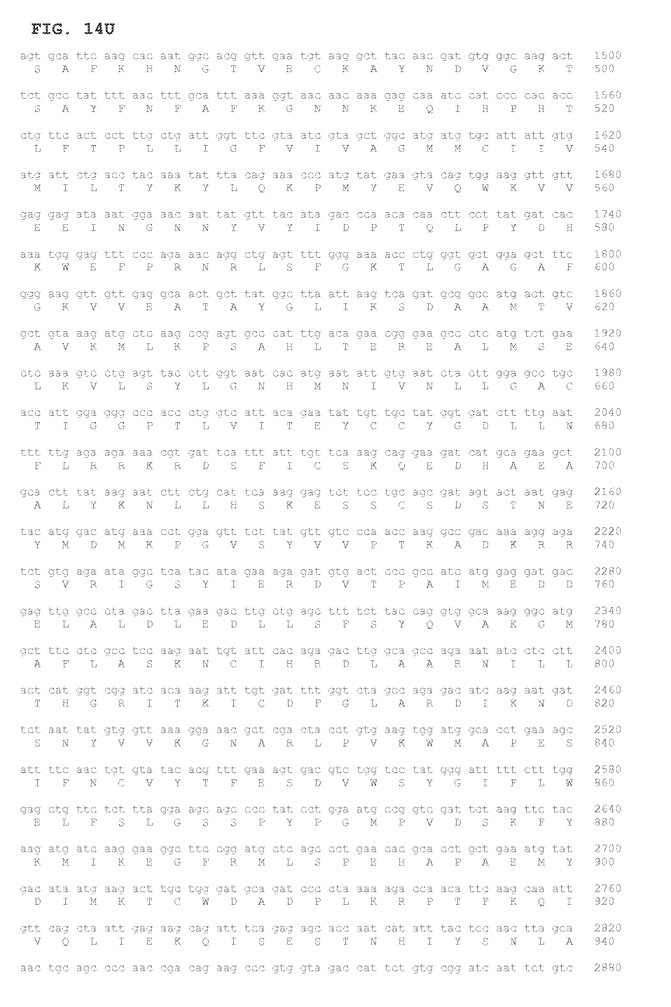

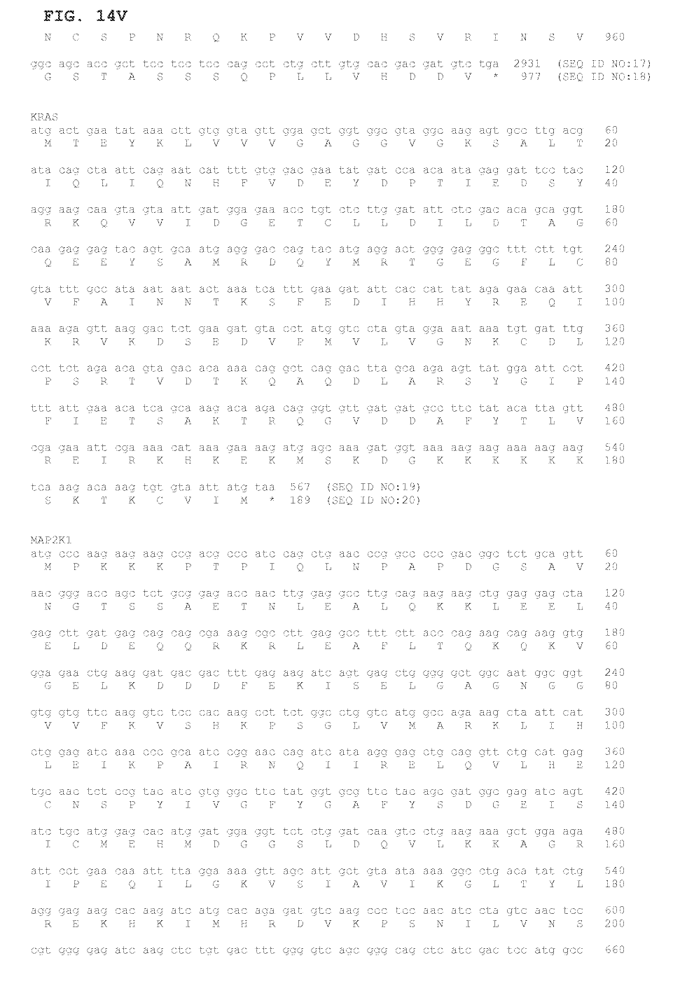

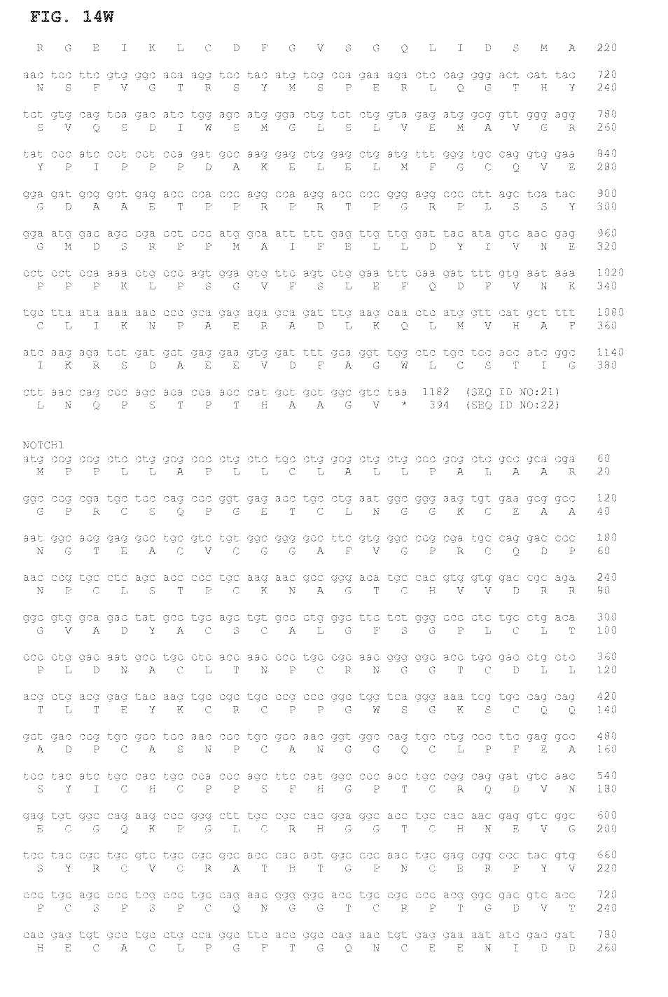

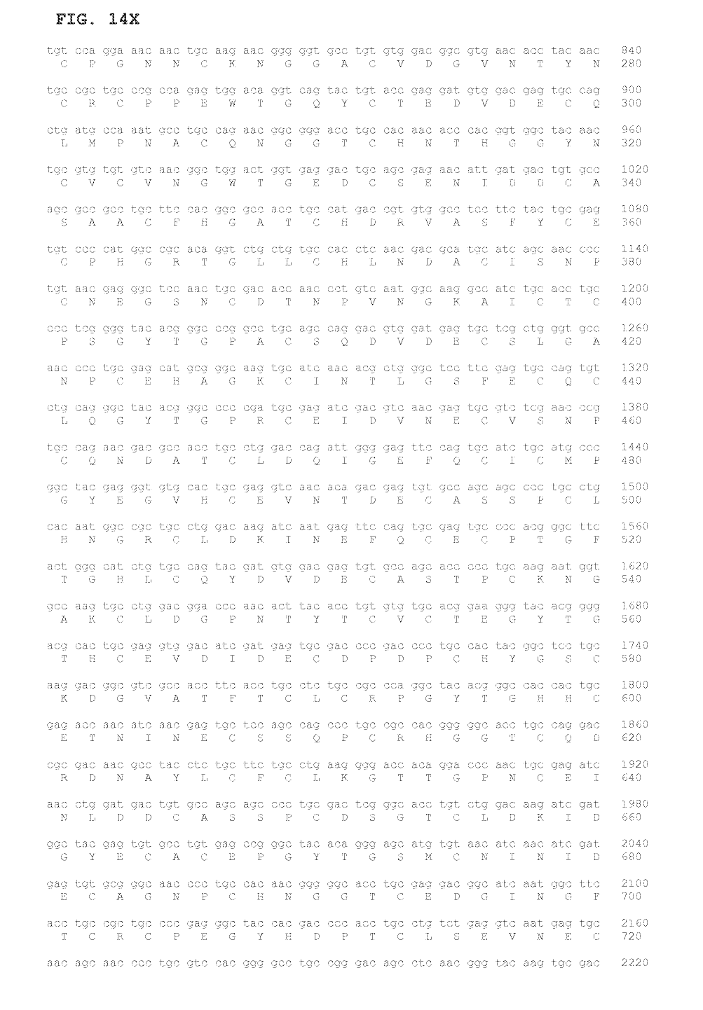

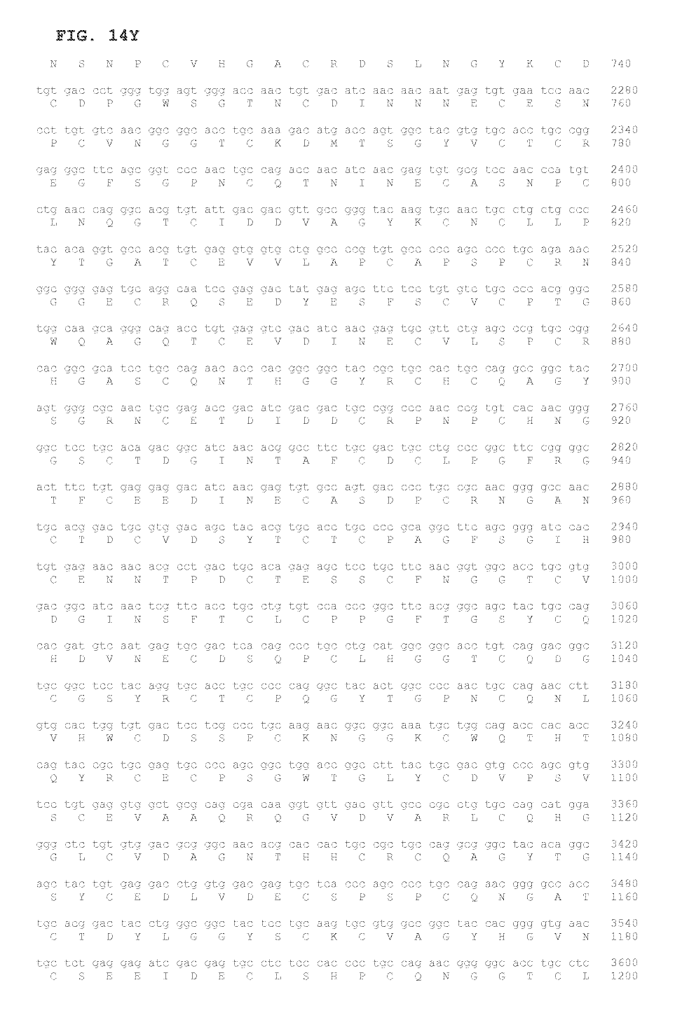

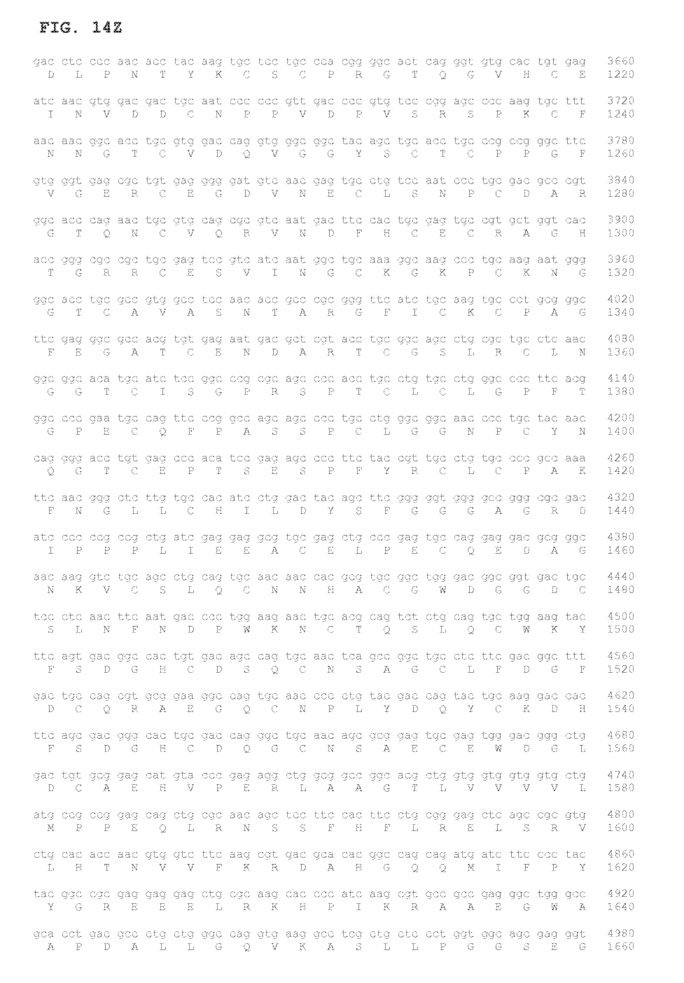

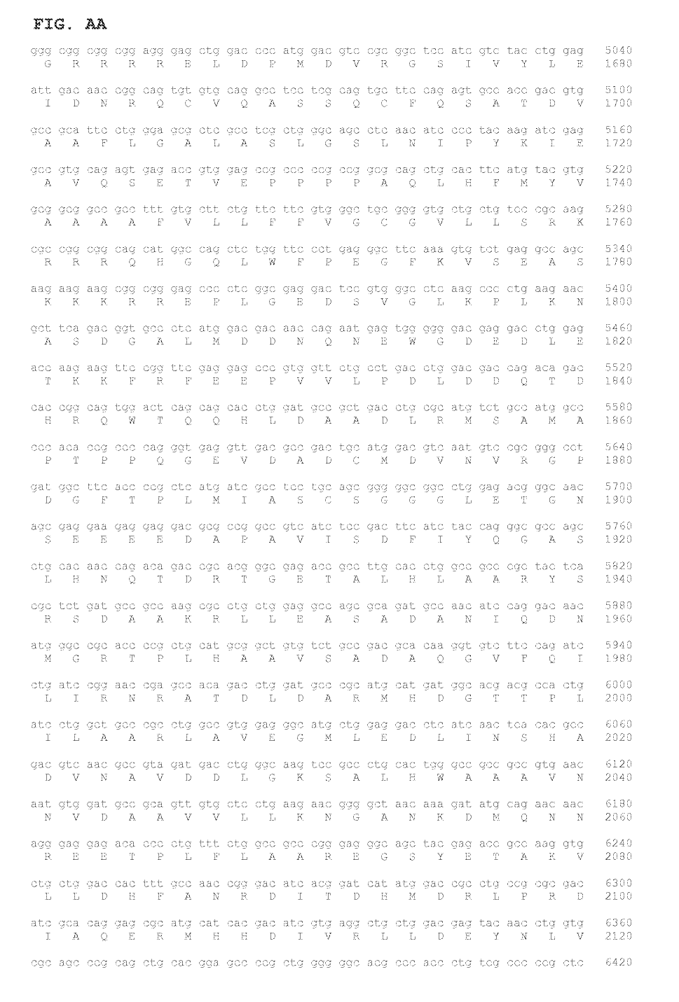

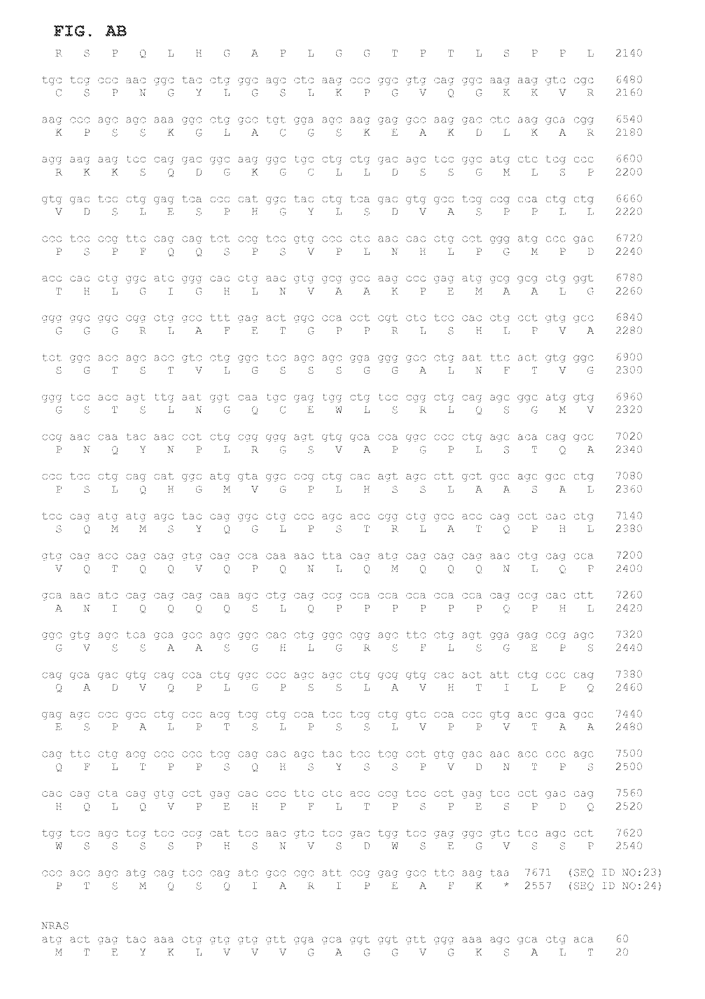

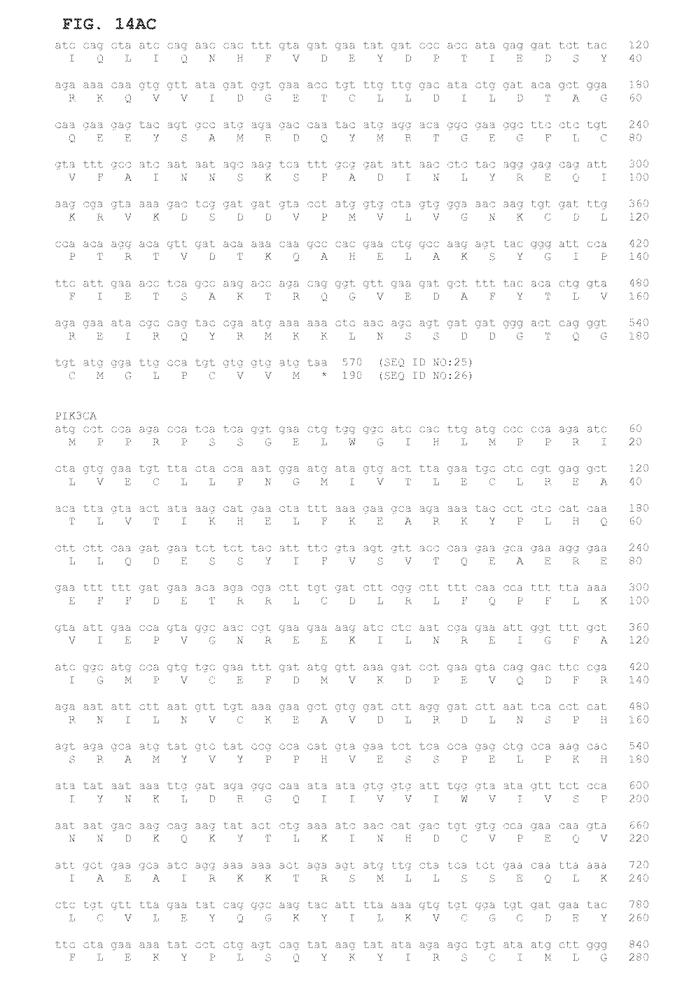

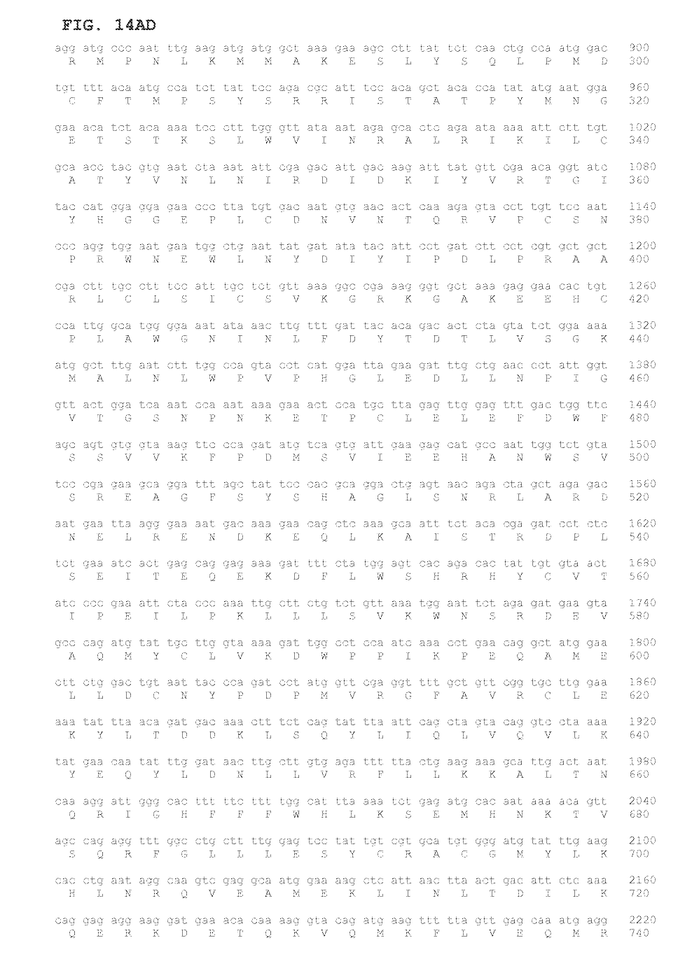

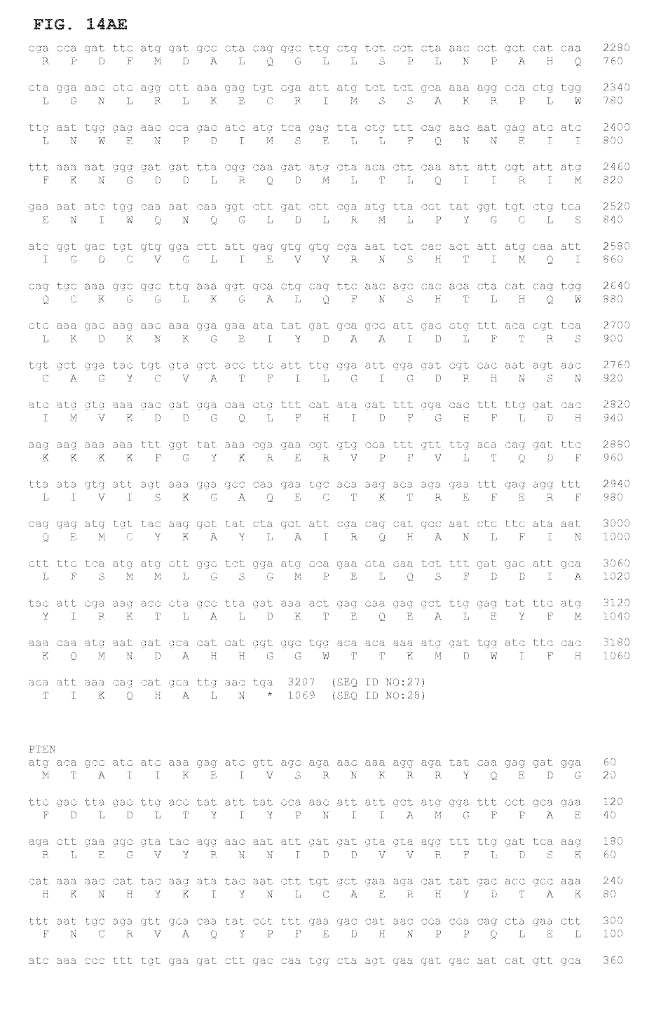

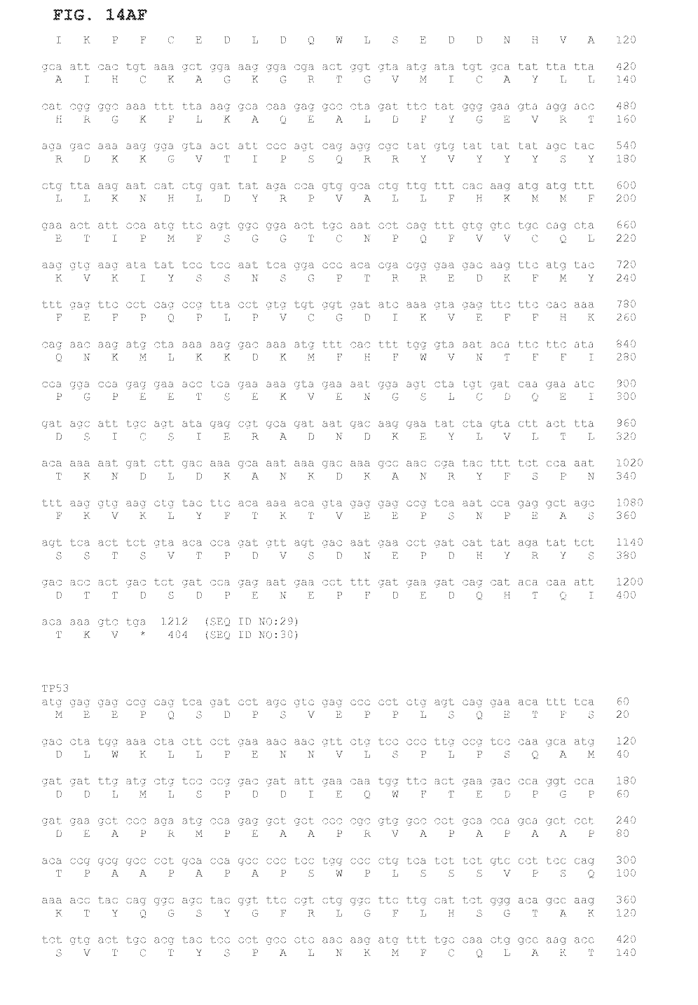

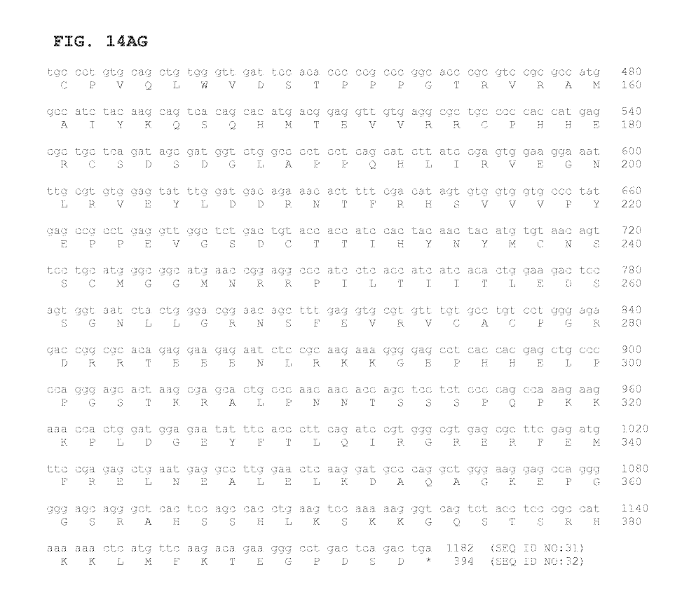

In one embodiment, the genotyping platform consists of nine multiplexed reactions that query 73 commonly mutated loci (Table 3A) within 16 key cancer genes (FIGS. 14A to 14AG). Since multiple nucleotide variants have been described at most of these sites, the test can detect over 120 previously described mutations (Table 3B).

In implementing this assay in a clinical setting, approximately two to three weeks are required from the time of test requisition until genotyping report finalization. This is referred to as a “real-time” assay, as oncologists ordering the test will have access to their patients' tumor mutational profiling data in time to influence clinical decision making. In these initial analyses, SNAPSHOT® results have substantially impacted therapeutic decisions. For lung cancer patients, detection of activating mutations in EGFR will identify patients most appropriate for first-line treatment with EGFR TKI therapy (Kobayashi et al. (2005) N Engl J Med 352, 786-792; Lynch et al. (2004) N Engl J Med 350, 2129-2139; Paez et al. (2004) Science 304, 1497-1500; Pao et al. (2004) Proc Natl Acad Sci USA 101, 13306-13311; Zhu et al. (2008) Cancer Lett 265, 307-317). Conversely, tumors harboring KRAS mutations are associated with lack of responsiveness to EGFR TKI treatment, and such patients are advised to pursue other therapeutic options (Pao et al. (2005b) PLoS Med 2, e17).

### Kits

Also described herein are kits for use in the present methods. For example, the kit can include a set of primers for detecting mutations in a biological sample; and a standard. The primers can be packaged in a suitable container, and can be in suitable combinations, e.g., Tables 8A and 8B. The kit can further comprise instructions for using the kit in the present methods.

The kit can also include a buffering agent, a preservative, or a protein stabilizing agent. The kit can also include components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

### EXAMPLES

The invention is further described in the following examples, which do not limit the scope of the invention described in the claims.

### Specimen Collection

A total of 250 primary cancer samples spanning 26 human malignancies were tested, which included: lung cancer (n=87), breast cancer (n=33), colorectal cancer (n=30), pancreatic cancer (n=23), prostate cancer (n=20), melanoma (n=11), chronic myeloproliferative disease (n=10), cholangiocarcinoma (n=6), gastric cancer (n=4), ovarian cancer (n=3), salivary gland cancer (n=3), and thyroid cancer (n=3) among others. Sixty-two of these primary tumor samples were evaluated for official clinical testing, and included 52 lung adenocarcinomas, most of them small core biopsies with very limited tissue. For hematopoietic malignancies, spare DNA that had been previously extracted from patient blood for clinical testing was obtained from the Massachusetts General Hospital (MGH) Molecular Diagnostics Laboratory. For solid tumors, formalin-fixed paraffin-embedded (FFPE) tumor blocks were obtained from MGH archives. Histological examination of hematoxylin and eosin-stained slides derived from FFPE samples was performed by a pathologist and assessed for the presence of a tumor. Available tumor tissue was manually macro-dissected from serial 5 μm unstained sections, or cored from the paraffin block using a 1.5 mm dermal punch. Total nucleic acid was extracted from FFPE material using a modified FormaPure System (Agencourt Bioscience Corporation, Beverly, Mass.) on a custom Beckman Coulter Biomek NXP workstation. Blood-derived DNA was extracted using the QIAamp Blood kit (QIAGEN Inc., Valencia, Calif.).

Assay Design and Validation

The COSMIC (Bamford et al. (2004) Br J Cancer 91, 355-358) database and PubMed was evaluated to select a panel of genes and loci previously reported to be frequently affected by somatic mutation in human cancer. Thirteen cancer genes were selected and 58 assays were designed to test for individual mutational events, which included: one insertion, three deletions and 52 substitutions (Tables 3A and 3B). Genomic position and sequencing information for all mutation sites were collected using the RefSeq gene sequences obtained using the human genome browser from the University of California Santa Cruz (UCSC), NCBI build 36.1. Primers for multiplexed PCR amplification were designed using Primer 3 software. Since FFPE tissue can be highly fragmented and of poor quality, design parameters restricted amplicon length to a maximum of 200 nt. All amplification primers (Table 7A) include a 10 nt long 5′ anchor tail (5′-ACGTTGGATG-3′ (SEQ ID NO: 236)) and the final PCR products range in length between 75 and 187 nt. The extension primer probes (Table 7B) were designed manually, according to the ABI PRISM SNAPSHOT® Multiplex Kit protocol recommendations (Life Technologies/Applied Biosystems, Foster City, Calif.) and using primer analysis tools available through the Primer 3 and Integrated DNA Technologies (IDT) web interfaces. Optimal conditions for multiplexed assays were determined empirically and are summarized in Table 8.

As part of the design rationale, assays covering four adjacent loci that are commonly mutated in the therapeutically relevant KRAS and NRAS oncogenes were included (nucleotide positions 34G, 35G, 37G and 38G were targeted for both genes). Due to the close proximity of these sites and to avoid compromising assay sensitivity due to primer competition, each nucleotide position was assayed in an independent panel. In addition, due to the extreme sequence similarity between KRAS and NRAS, to avoid non-specific results, the assays for these two genes were segregated into individual multiplexed reactions. Eight panels were populated with the 58 assays outlined in Table 3. Many of these genes and assays are clinically relevant. In addition, since the costs of running the assay (regarding tumor material and the actual price per assay) are mainly dictated by the number of panels, a set of common mutations affecting critical cancer genes for which a therapeutic agent is still currently unavailable was also included. The addition of these mutations is useful in a clinical setting, as they may correlate with a better or worse prognosis or to influence response to specific therapies, and thus contribute to better cancer care in the future.

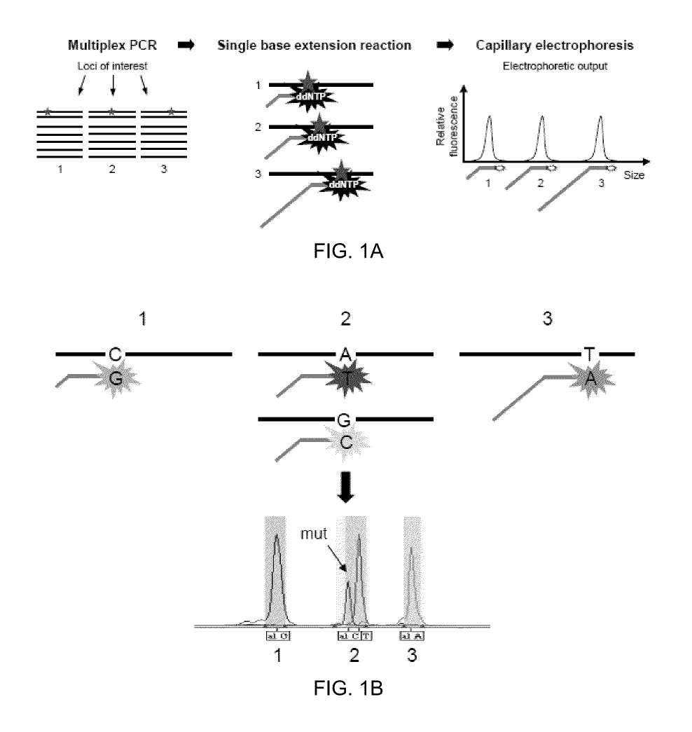

In order to develop a robust assay for clinical tumor genotyping, several high-throughput platforms were evaluated for the ability to detect low-level mutations in DNA extracted from FFPE tissues. The SNAPSHOT® assay from Applied Biosystems consisting of a multiplexed PCR step followed by a single-base extension reaction that generates allele-specific fluorescently labeled probes (FIG. 1) was ultimately selected given its low background noise, high sensitivity, and good performance with FFPE-derived DNA in a multiplexed setting. Moreover, genetic analysis using the SNAPSHOT® methodology follows a simple workflow, with the only major instrumentation requirement being a capillary electrophoresis automated DNA sequencer. The SNAPSHOT® system is particularly attractive because virtually all clinical laboratories already have at least one of these sequencers, hence avoiding additional capital expenses and facilitating rapid implementation by clinical testing sites.

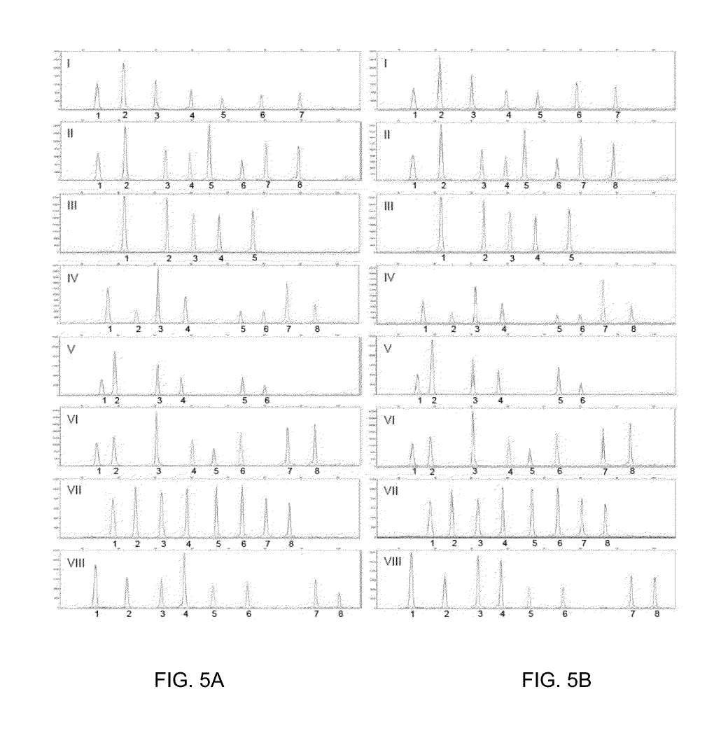

Assays were designed to detect recurrent mutations in some of the most important cancer genes, many of which activate cancer signaling pathways that are currently targeted by either FDA-approved therapies or by agents in advanced stages of clinical development (Table 1). The genotyping platform consists of eight multiplexed reactions that query 58 commonly mutated loci within 13 key cancer genes. Since multiple nucleotide variants have been described at most of these sites, the test can detect 120 previously described mutations (Table 3). The assay is focused predominantly on oncogenes because aberrantly activated oncogenes are preferential targets for pharmacologic inhibition, and gain-of-function mutations in oncogenes are usually limited to a small set of codons. Accordingly, the assay captures 94% to 99% of the mutation frequency described for the BRAF, KRAS, and JAK2 oncogenes, which are frequently mutated in a very few hotspots. Representative spectra of all eight SNAPSHOT® genotyping panels are depicted in FIG. 5, which illustrates the performance of the assay with both high-quality, commercially available genomic DNA (A) and total nucleic acid extracted from FFPE primary tumor tissue from patients (B).

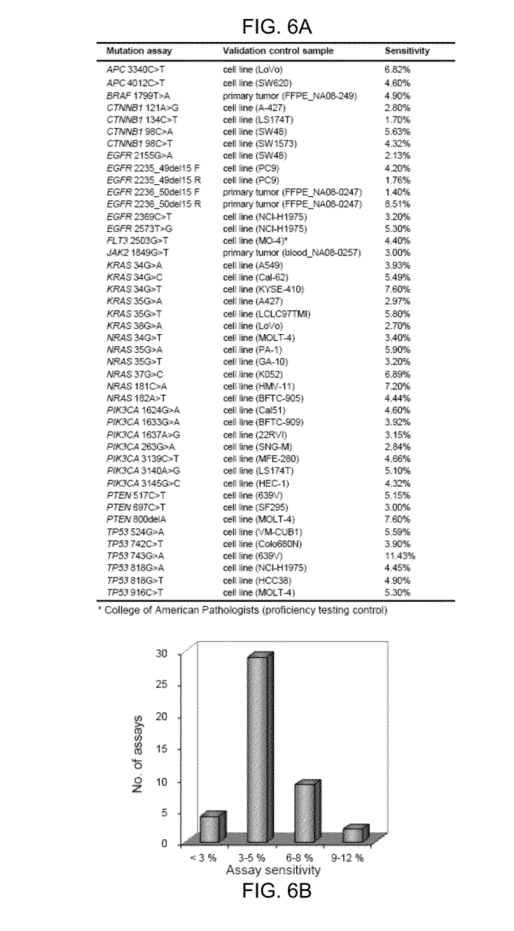

Assay validation was carried out with control DNA harboring the mutations of interest, which included: primary tumor DNA, cancer cell line DNA, and custom-designed synthetic oligonucleotides (Table 3). All SNAPSHOT® assays identified the expected mutations. In addition, allele-specific assays that could be validated using genomic DNA were assessed for sensitivity, which ranged from 11.4% to 1.4% and was on average approximately 5% (FIG. 6), an improvement over direct sequencing that is reported to have a sensitivity of about 20% (Hughes et al. (2006) Blood 108, 28-37). Since allele-specific detection methods test a sequence change at one site, the sensitivity of each assay is not affected by the mechanism that caused the mutation (point mutation vs. insertion or deletion). The sensitivity data summarized in FIG. 6 includes 44 assays (39 point mutations and 5 deletions) and the average sensitivity for the deletions (4.69%) was very similar to the average sensitivity for all assays (4.64%).

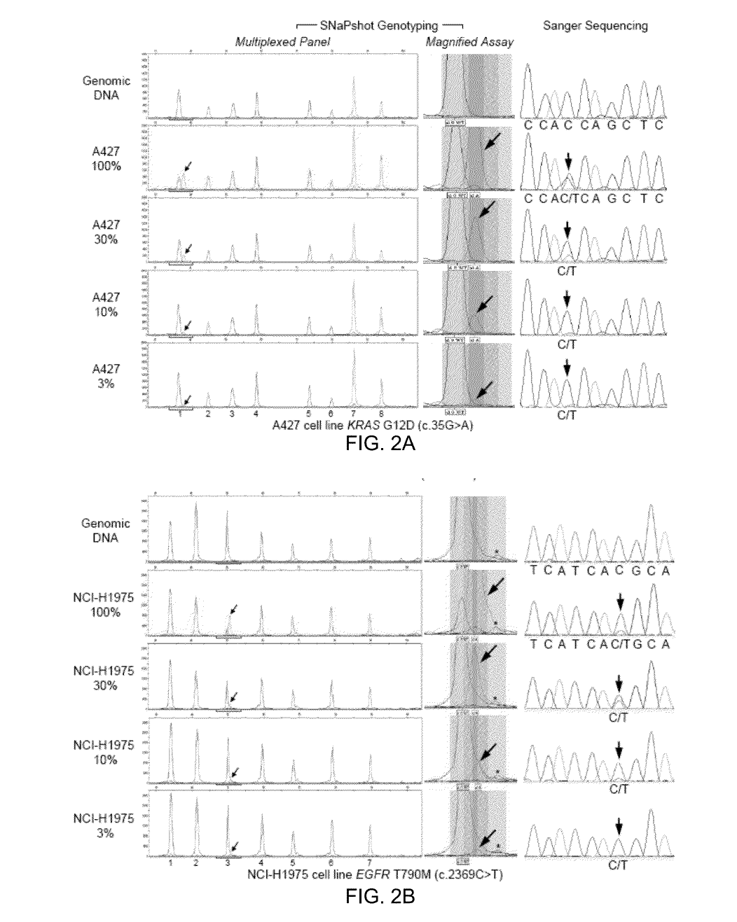

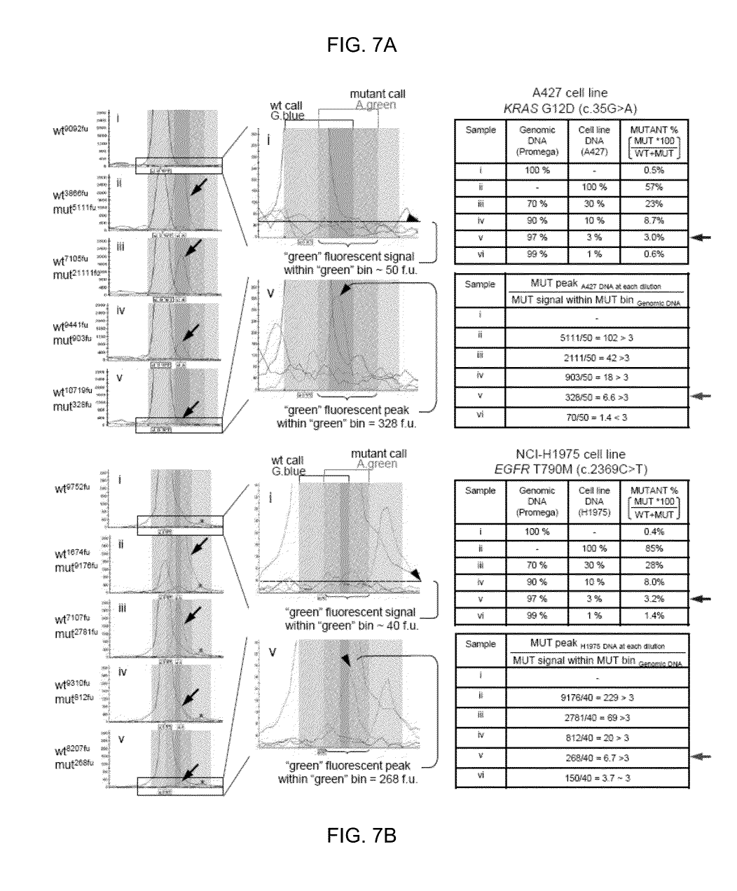

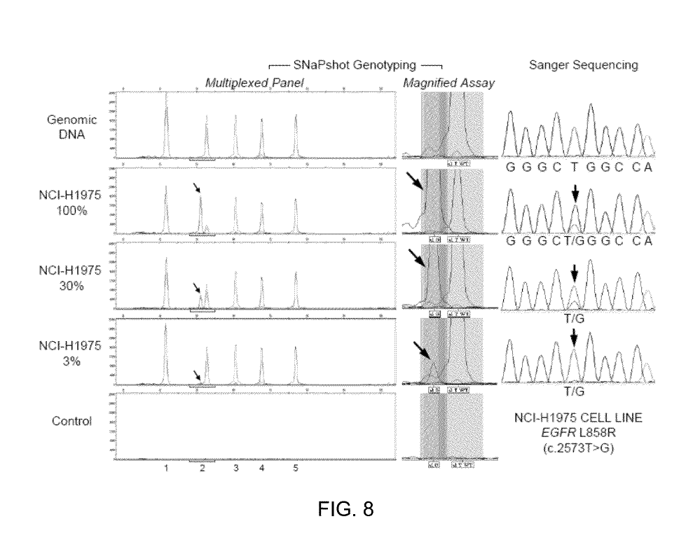

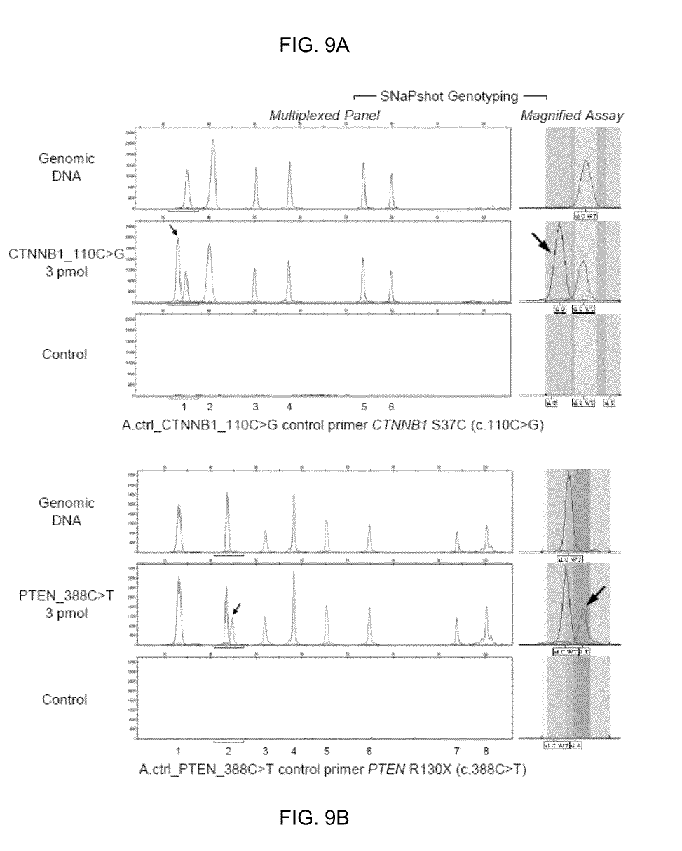

As an example of validation and sensitivity testing, FIG. 2 illustrates an analysis for two clinically relevant mutations, KRAS G12D and EGFR T790M, both of which confer resistance to anti-EGFR therapy. In each case, sensitivity was determined using DNA from a cancer cell line harboring the mutation of interest, serially diluted with commercially available wild-type DNA. The A427 lung carcinoma cell line was used to detect the highly prevalent KRAS G12D mutation (FIG. 2A) (Bamford et al. (2004) Br J Cancer 91, 355-358) and the NCI-H1975 lung adenocarcinoma cell line was used to identify the EGFR T790M mutation (FIG. 2B), which represents the most commonly described mechanism of acquired resistance to EGFR TKIs in lung cancer (Ladanyi and Pao (2008) Mod Pathol 21 Suppl 2, S16-22; Pao et al. (2005a) PLoS Med 2, e73). In both instances, assay sensitivity was approximately 3% and data quality was very comparable to traditional Sanger sequencing analysis (panels on the right). A detailed illustration of the process used to calculate assay sensitivity for these two cases is shown in FIG. 7. Of note, the use of fluorescently labeled probes in the SNAPSHOT® assay enables allele recognition to be contingent on two parameters: slightly different masses and distinct color readouts. These features facilitate the ability to distinguish low-level mutations from background noise. Finally, while 75% of the assays (33 out of 44) shown in FIG. 6 were highly sensitive detecting levels of mutant allele of ≦5%, a mutant allele cutoff of 10% was typically used when analyzing samples of unknown genotype, which is a conservative value to confidently call a mutation (detailed scoring guidelines are provided herein). Additional sensitivity data and examples of assay validation using synthetic oligonucleotide probes are illustrated in FIGS. 8 and 9.

### Tumor Genotyping

The Applied Biosystems (ABI) PRISM® SNAPSHOT® Multiplex system was originally developed to detect single nucleotide polymorphisms (SNPs) (Lindblad-Toh et al. (2000) Nat Genet 24, 381-386) (FIG. 1). Multiplexed PCR was performed in a volume of 10 μl, containing 0.5 units of Platinum Taq polymerase (Invitrogen, Carlsbad, Calif.), 30 nmol of MgCl₂, 3 nmol of dNTPs (Invitrogen, Carlsbad, Calif.), amplification primers (IDT, Coralville, Iowa) as specified in Table 8A, and ideally either 20 ng of genomic DNA or 60 ng of total nucleic acid. When the amount of tissue was limiting, multiplexed PCR was performed with as low as 5 ng of total nucleic acid. Thermocycling was performed at 95° C. for 8 min, followed by 45 cycles of 95° C. for 20 s, 58° C. for 30 s, and 72° C. for 1 min, and one last cycle of 72° C. for 3 min. Excess primers and unincorporated dNTPs were inactivated using 3.3 units of shrimp alkaline phosphatase (USB, Cleveland, Ohio) and 2.7 units of exonuclease I (USB, Cleveland, Ohio) for 60 min at 37° C., followed by 15 min at 75° C. for enzyme inactivation. The primer extension reaction was performed in a volume of 10 μl, containing 3 μl of PCR product, 2.5 μl of SNAPSHOT® Multiplex Ready Reaction mix, and the appropriate cocktail of PAGE-purified extension primers (IDT) (Table 8B). Cycling conditions were 96° C. for 30 s, followed by 25 cycles of 96° C. for 10 s, 50° C. for 5 s, and 60° C. for 30 sec. After treatment with 2 units of shrimp alkaline phosphatase, 0.5 μl of labeled extension products were mixed with Hi-Di Formamide and 0.2 μl of GeneScan-120LIZ size standard (Life Technologies/Applied Biosystems) to a final volume of 10 μl. Following denaturation at 95° C. for 5 min, the extension products were resolved by running on 36 cm long capillaries in an automatic sequencer (ABI PRISM 3730 DNA Analyzer, Life Technologies/Applied Biosystems), according to the SNAPSHOT® default settings established by ABI. Data analysis was performed with GeneMapper Analysis Software version 4.0 (Life Technologies/Applied Biosystems) using the automatic calling parameters described herein.

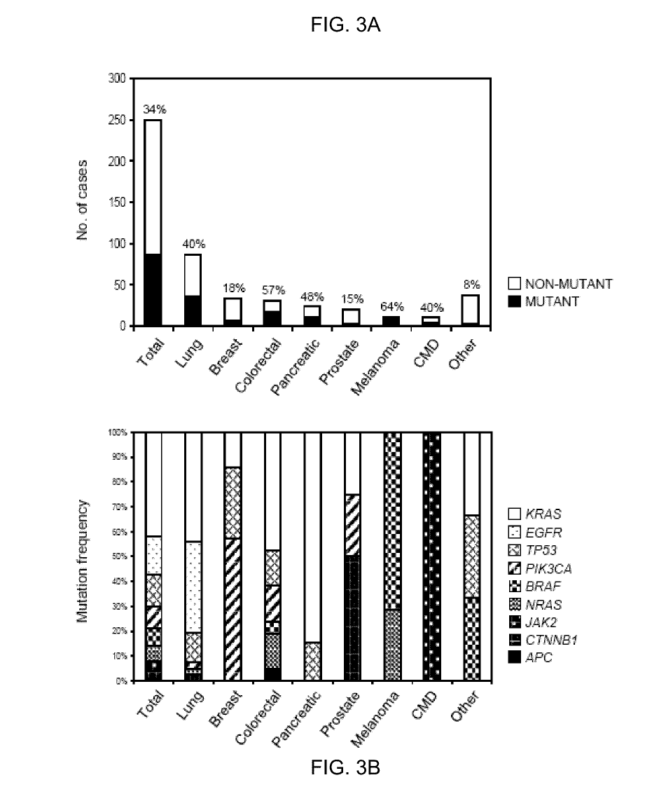

Two hundred fifty primary cancer samples representative of major human malignancies were profiled, and a total of 100 mutations were detected in 86 (34%) of the cases (Table 4). Of note, the majority of these tumor samples (96%) were derived from FFPE tissue. The most frequently mutated gene was KRAS, across multiple tumor types, followed by EGFR, which was detected in lung adenocarcinomas (Table 2 and FIG. 3). Consistent with previous reports (Subramanian and Govindan (2008) Lancet Oncol 9, 676-682), KRAS mutations in lung cancer were strongly associated with a history of smoking (89% of KRAS mutations were found in patients that smoked >10 packs/year), while the reverse was true for EGFR, with 73% of EGFR-mutant tumors originating from patients who had never smoked.

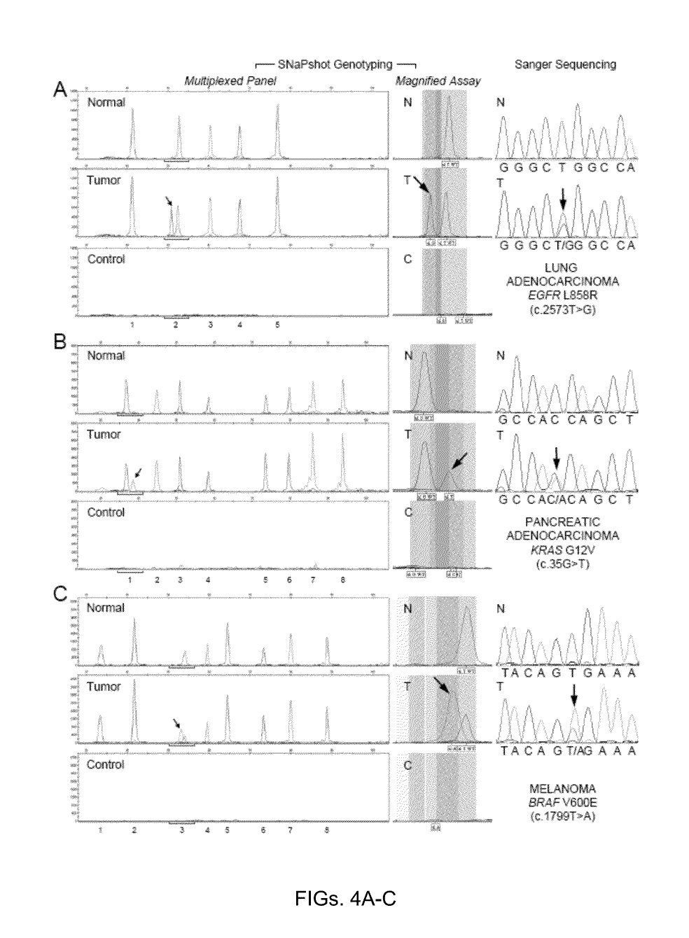

The specificity of SNAPSHOT® genotyping was evaluated by analysis of primary tumor samples and matching normal tissue from the same individual. FIG. 4 includes examples of adenocarcinomas of the lung (4A) and pancreas (4B), and of malignant melanoma (4C), and depicts the most prevalent activating mutations in the data set for EGFR (L858R), KRAS (G12V), and BRAF (V600E), respectively. The mutant allele (arrow) is only detected in the tumor specimen and not in the matching normal tissue, demonstrating the specificity of the test.

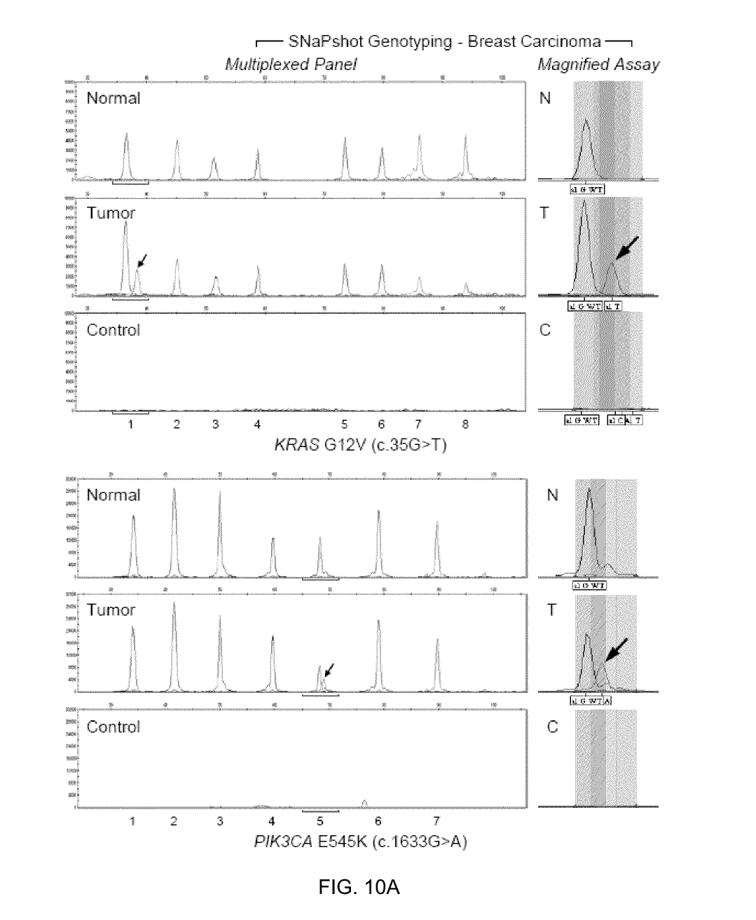

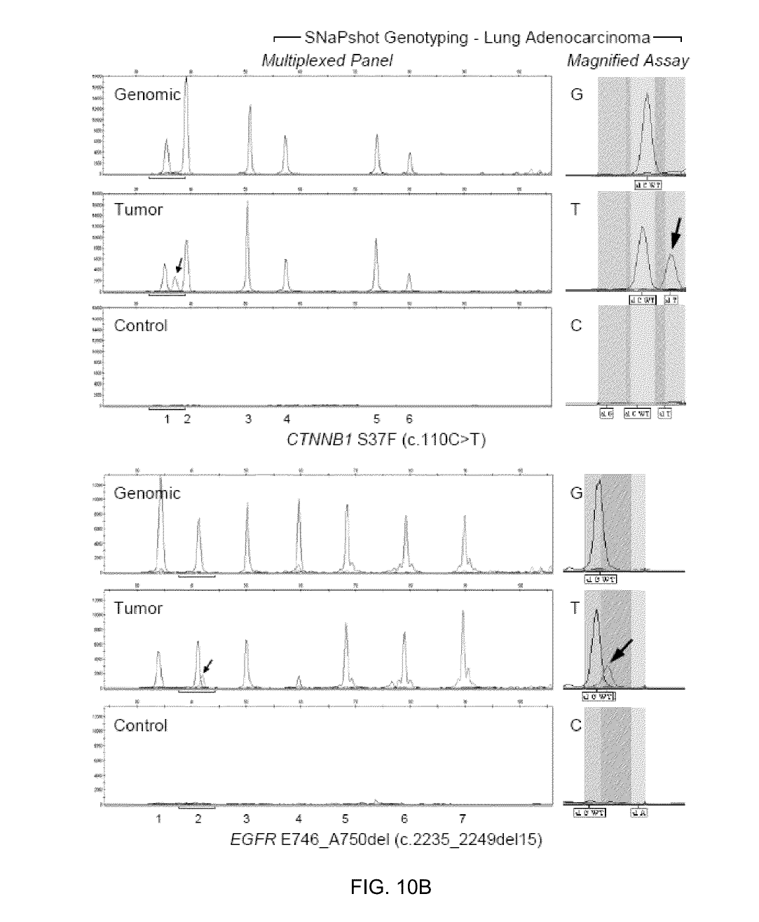

In general, the genotyping results were consistent with the documented mutational prevalence for oncogenes, but lower than expected mutational frequencies were observed for tumor suppressors (Table 5). Slight discrepancies between these observations and the reported mutation frequencies for oncogenes included lower than expected mutation prevalences for beta-catenin (CTNNB1) and BRAF in pancreatic and colorectal tumors, respectively; and higher than the reported frequencies for NRAS in colorectal cancer. Surprisingly, the incidence of NRAS mutations in the colorectal cancer population tested was three-fold higher than previously described. Interestingly, a number of mutations and combination of mutations (marked by the asterisks in Table 2) were identified that are rare or not previously described in the respective tumor types. Some of these less common events are illustrated in FIG. 10 and include the co-occurrence of activating mutations in KRAS and PIK3CA in breast cancer, which were proposed to be mutually exclusive events based on cell line studies (Hollestelle et al. (2007) Mol Cancer Res 5, 195-201), and of beta-catenin and EGFR mutations in a rarely recognized case of fetal-type lung adenocarcinoma (Nakatani et al. (2002) Mod Pathol 15, 617-624).

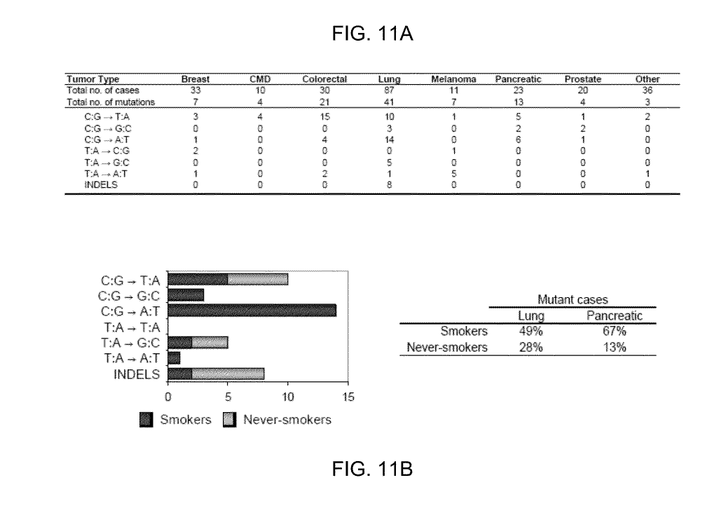

Within the subset of events captured by the panel, the observations were consistent with previous findings from genome-wide studies (FIG. 11). The most common mutations observed in colorectal cancer were C:G to T:A transitions, previously shown to be abundant in this tumor type and a possible effect of dietary carcinogens (Sjoblom et al. (2006) Science 314, 268-274). Moreover, consistent with previous reports, C:G to A:T transversions (34%) and C:G to T:A transitions (24%) were identified as the most frequent mutation classes in lung cancer (Ding et al., 2008). C:G to A:T transversions have been associated with smoking and are thought to be induced by tobacco smoke carcinogens (Slebos et al. (1991) J Natl Cancer Inst 83, 1024-1027). All C:G to A:T transversions detected in the lung cancer population were found in smokers (FIG. 11B), which is likely in part due to the pattern of KRAS mutations commonly seen in smokers. Finally, a higher proportion of mutations were identified in smokers than in never-smokers for lung (49% vs. 28%) and pancreatic (67% vs. 13%) cancers, in agreement with previously observed correlations between smoking and the number of genetic changes in these tumor types (Blackford et al. (2009) Cancer Res 69, 3681-3688; Ding et al. (2008) Nature 455, 1069-1075).

### Sequencing Analysis

Traditional Sanger sequencing was performed in a volume of 20 μl, containing 1 unit of Taq polymerase (Invitrogen, Carlsbad, Calif.), 4 nmol of dNTPs (Invitrogen, Carlsbad, Calif.), 10 pmol of forward (a1) and reverse (a2) primers, 40 nmol of MgCl₂ (or the amount indicated in Table 10), and either 40 ng of genomic DNA or 120 ng of total nucleic acid. Initially, sequencing was attempted with the same amplification primers and cycling parameters used for SNAPSHOT® multiplexed PCR. For those cases where this strategy was not successful, new primers were designed (Table 10) and the cycling conditions were: 94° C. for 5 min, followed by 38 cycles of 94° C. for 30 s, a specific annealing temperature for 30 s and 72° C. for 45 sec, and one last cycle of 72° C. for 10 min. The annealing temperature and amount of MgCl₂ used for each PCR are detailed in Table 10. The resulting PCR products were treated using 1 unit of shrimp alkaline phosphatase (USB, Cleveland, Ohio) and 5 units of exonuclease I (USB, Cleveland, Ohio) at 37° C. for 20 minutes followed by 80° C. for 15 minutes, and tested for the presence of mutations by bi-directional Sanger sequencing using the BigDye Terminator V1.1 Cycle Sequencing Kit (Applied Biosystems), according to the manufacturer's recommendations. The sequencing reaction step was performed with the original PCR primers or with the incorporated M13 tags. Tumor and control human genomic DNA (Promega, Madison, Wis.) sequences were compared using the AB Sequencing Analysis Software v5.2 (Applied Biosystems).

### EGFR Exon 19 Sizing Assay

A PCR-based strategy was developed to identify insertions or deletion mutations in exon 19 of the EGFR gene, which is a hotspot region for deletions. Amplification primer sequences were as follows, with the forward primer being 5′-labeled with the NED fluorophore: NED-EGFR_Ex19_F [0.1 μM]: 5′-NED-GCACCATCTCACAATTGCCAGTTA-3′ (SEQ ID NO:234); EGFR-Ex19-REV1 [0.1 μM]: 5′-AAAAGGTGGGCCTGAGGTTCA-3′ (SEQ ID NO:235). 20 ng of DNA template was amplified using Platinum Taq polymerase in the presence of 2 mM MgCl₂ (Invitrogen, Carlsbad, Calif.). The 20 μl reaction was subjected to 5 minutes of denaturation at 94° C. and 40 cycles of denaturation at 94° C. for 30 seconds, annealing at 60° C. for 30 seconds, and elongation at 72° C. for 60 seconds. Following PCR amplification, products were diluted 1:30 in water and a 1 μl aliquot was added to 9.9 μl of Hi-Di Formamide and 0.1 μl of GeneScan 500 LIZ Size Standard (Applied Biosystems Inc, Foster City, Calif.). Heat-denatured samples were analyzed through capillary electrophoresis using the automated ABI 3730 DNA Analyzer with GeneMapper software (Applied Biosystems Inc). Insertions or deletions were visualized by shifts in molecular weight of the fluorescently-identifiable PCR amplicon relative to wild-type.

### Data Analysis

Panels and bin set parameters for automatic data analysis were created using GeneMapper Software version 4.0, according to the manufacturer's instructions and are provided herein. Briefly, for each genetic locus tested by a SNAPSHOT® mutation assay, there are four possible alleles (for deletion and insertion assays only two alleles were considered: the wild-type allele and the expected nucleotide change resulting from the specified deletion or insertion). The position of each of these alleles can be automatically captured by the analysis software upon the creation of specific bins (allele definitions). Bin parameters for each assay were initially established using Primer Focus Kit data (Life Technologies/Applied Biosystems) according to the manufacturer's recommendations and were subsequently adjusted using reference data from wild-type tumor samples and from the mutant controls used for assay validation. The panel and bin set parameters used in this study are provided herein. Automatic mutation calling was set at a 5% sensitivity threshold. Interpretation of SNAPSHOT® genotyping results was accomplished by automatic analysis of the raw data using the established panels and bin settings, followed by visual inspection of the spectra for all loci by at least two users. In addition, if a mutation was detected, a third user reviewed the panel containing the mutation. Since spectral analysis follows a very strict set of scoring guidelines (described below), the concordance in calling between different users was extremely high.

Data analysis was performed using the following scoring criteria.

Pass. For each sample, an individual SNAPSHOT® assay passed if: (1) the peak fluorescent height for the wild type allele was ≧1,000 f.u. (this value was selected for being approximately 50-100 times higher than the overall background noise, however, since signal intensities may vary for different genetic analyzer instruments, this value should be adjusted by different users); and (2) the peak fluorescent height for the wild type allele in the negative control (water sample) was <10% of the height of the wild type allele in the clinical sample.

Mutant. A mutation was called for a specific assay when: (1) the % of mutant allele for one of the 3 possible nucleotide variants, falling within its corresponding bin, was ≧10% (fluorescent peak height ratio of [mutant/(mutant+wild type)] alleles>0.10), and (2) the peak fluorescence of the mutant allele was >3 times above the background in the wild type control sample (FIG. 7). Lower level mutations were also called if the % of mutant allele was ≧5% and the peak fluorescence of the mutant allele was >5 times above background. For all suspected mutant samples, the SNAPSHOT® panel containing the assay in question was repeated to confirm the initial result.

Repeat. A specific panel was repeated if it contained an assay with a suspected mutation, or if it contained an assay that failed (either because: (1) the peak fluorescent height for the wild type allele was <1,000 f.u. or (2) the negative control produced a peak fluorescent height for the wild type allele that was ≧10% of the height of that same peak in the test sample).

### Assay Validation and Sensitivity Assessment

The tumor genotyping assay described in this example consists of 8 SNAPSHOT® multiplex panels that test for 58 commonly mutated loci in 13 cancer genes. Since multiple nucleotide variants have been described at most of these loci, the assay can detect 120 previously described mutations (Table 3). The frequency of occurrence of each allele variant was calculated using data compiled by the Wellcome Trust Sanger Institute and reported for each cancer gene in the COSMIC database (Bamford et al. (2004) Br J Cancer 91, 355-358) (v42 release). To calculate the frequencies of gene mutation depicted in Tables 1 and 3, all mutations described in the COSMIC database with available positional information at the amino acid level were included.

Eighty-one out of the 120 allele variants covered by our panel were validated, using three types of control samples (Table 3): (1) whenever possible, primary tumor samples that had been previously tested at the MGH Molecular Diagnostics Pathology Laboratory were used and shown to carry the mutations of interest; (2) for the majority of the assays, validation was performed using cancer cell lines harboring known mutations, which were identified using the Wellcome Trust Sanger Institute Cancer Cell Line Project database; and (3) synthetic oligonucleotides harboring the mutation of interest were designed to validate those allele variants for which an appropriate tumor sample or cancer cell line control were not identified (Table 9).

Genomic DNA was extracted from blood using the QIAamp Blood kit (QIAGEN Inc., Valencia, Calif.), or from FFPE primary tumor tissue and frozen cancer cell line pellets using the RecoverALL™ Total Nucleic Acid Isolation Kit (Applied Biosystems, Foster City, Calif.), according to the manufacturer's recommendations. To prepare the synthetic control samples, 1 to 40 pmol of custom-made oligonucleotides designed to include the mutation of interest, were added to 3 μl of PCR product obtained from amplification of 20 ng of male genomic DNA (Promega, Madison, Wis.) as indicated in Table 9, followed by Exo/SAP treatment and by the extension reaction. Each mutant sample was tested using the SNAPSHOT® genotyping panel containing the assay to be validated, and male genomic DNA (Promega, Madison, Wis.) was used as a wild-type control for each run.

For those allele-specific assays that could be validated using genomic DNA derived from primary tumor tissue or from cancer cell lines, a sensitivity assessment was also performed (FIG. 6). For sensitivity testing, mutant DNA samples were serially diluted in 1:3 increments with male genomic DNA (Promega), to obtain solutions of 100%, 30%, 10%, 3%, and 1% of mutant DNA input material.

It is well established that cancer cells are prone to genetic instability, which can result in the gain or loss of genetic material. In addition, primary tumor specimens may contain normal (non-cancerous) cells. Due to this heterogeneity, the calculated amount of input mutant DNA material does not accurately reflect the relative amount of mutant vs. wt allele in each tested sample. Thus, the percentage of mutant allele in each sample was calculated by comparing the fluorescent peak heights of the mutant and wild-type alleles, according to the following: % mutation=[mutant allele peak height/(wild-type allele peak height+mutant allele peak height)]*100.

The sensitivity of each assay was established as the lowest % mutation for which the fluorescent peak height of the mutant allele is >3× background (the background for a specific mutant allele is defined as the height of the fluorescent peak corresponding to that allele, within its assigned bin in the wild type control genomic DNA sample). For a detailed explanation of the process involved in sensitivity assessment, please refer to FIG. 7.

### Independent Confirmation of Test Results

All of the mutations detected in a primary tumor sample were initially verified by an independent SNAPSHOT® reaction using the genotyping panel containing the assay in question. The cases of chronic myeloproliferative disease and a small number of colorectal adenocarcinomas had been previously sequenced for JAK2 exon 12 and for KRAS exon 2, respectively, as part of standard clinical testing. Once genotyping analysis was completed, the SNAPSHOT® results were confirmed to match the previous clinical findings. The additional mutations were evaluated using standard Sanger sequencing. In total, 90% of the mutations identified by SNAPSHOT® genotyping were independently confirmed (inability to independently verify the presence of mutation in 10% of the cases was due to unsuccessful Sanger sequencing data, as a result of limiting amounts of nucleic acid).

Mutational profiling of 250 primary tumor samples identified a total of 100 mutations that could be classified into 33 distinct mutation groups. Attempts to identify cases with normal matching tissue for each of these 33 independent mutation types, and perform a side-by-side comparison between tumor and normal tissue from the same individual, to test the specificity of the SNAPSHOT® assay were conducted for 25 out of the 33 mutation types (76%). In all cases, the somatic mutant allele was only detected in the tumor specimen and not in the matching normal tissue, which confirmed the specificity of the corresponding SNAPSHOT® assays.

### Clinical Application of Genetic Profiling

Out of all primary tumors examined, 62 cases were genotyped as part of what has now become routine clinical testing (Table 4). Exon 19 of the EGFR gene is a hotspot for in-frame deletions, often found in lung cancer and that have been associated with response to EGFR TKI therapy (Lynch et al. (2004) N Engl J Med 350, 2129-2139; Mok et al. (2009) N Engl J Med 361, 947-957; Paez et al. (2004) Science 304, 1497-1500; Pao et al. (2004) Proc Natl Acad Sci USA 101, 13306-13311). Although the assay described herein tests for the two most common deletions in the EGFR intracellular domain, due to the therapeutic implications of this region, mutational profiling of clinical cases was complemented by a PCR-based sizing assay designed to capture all deletions (or insertions) in EGFR exon 19. For most cases (98%) there was concordance between the SNAPSHOT® results and the exon 19 sizing data, however, the second approach identified one additional deletion in EGFR which was not captured by SNAPSHOT® genotyping (Table 4).

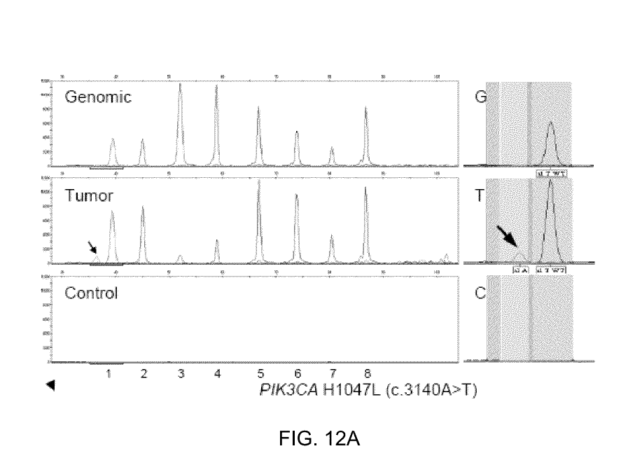

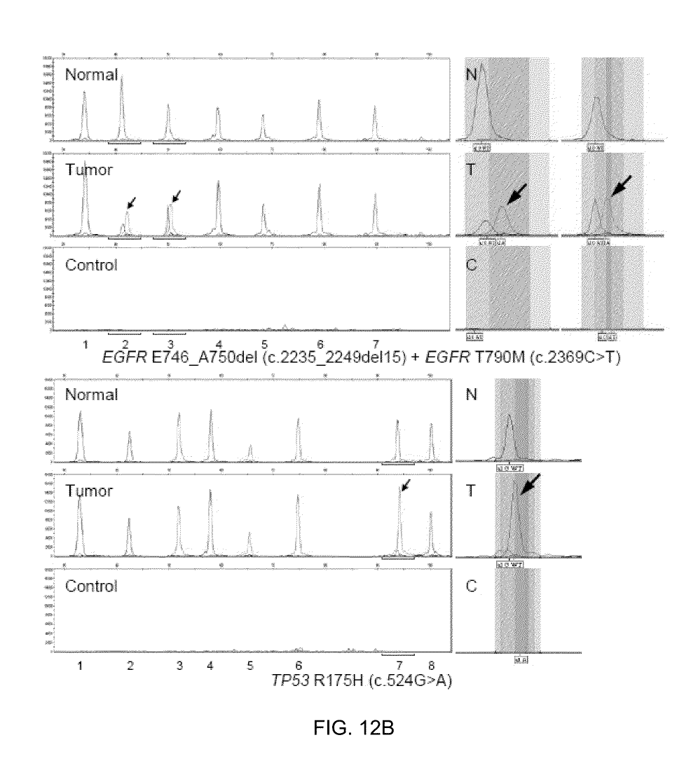

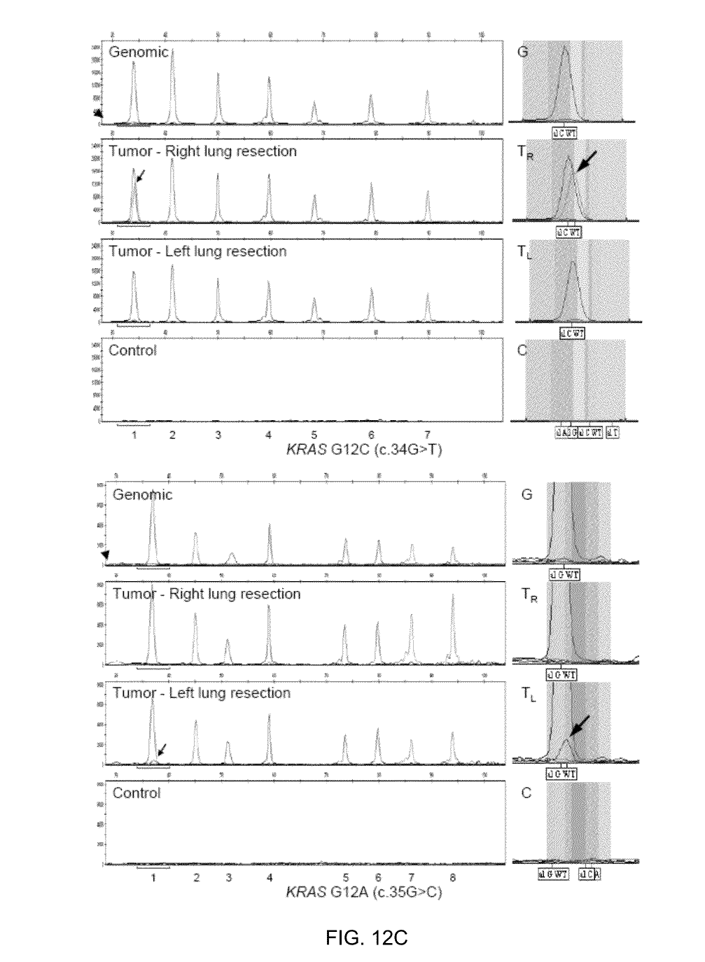

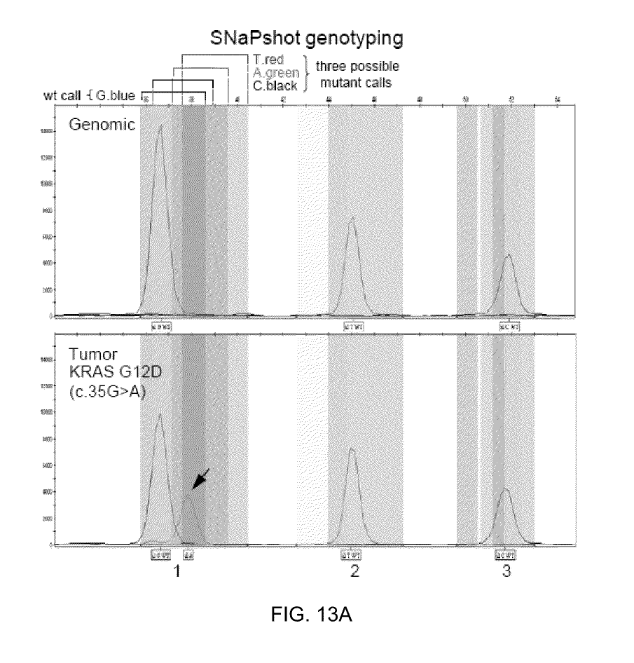

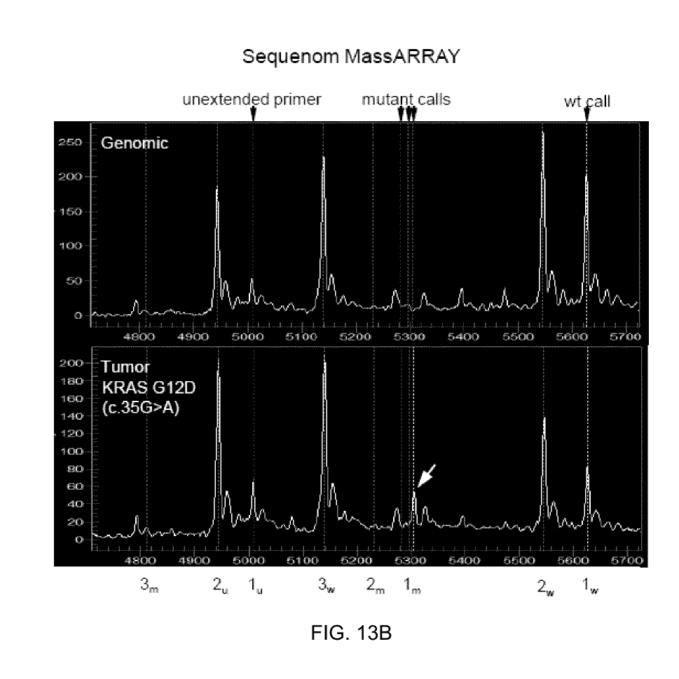

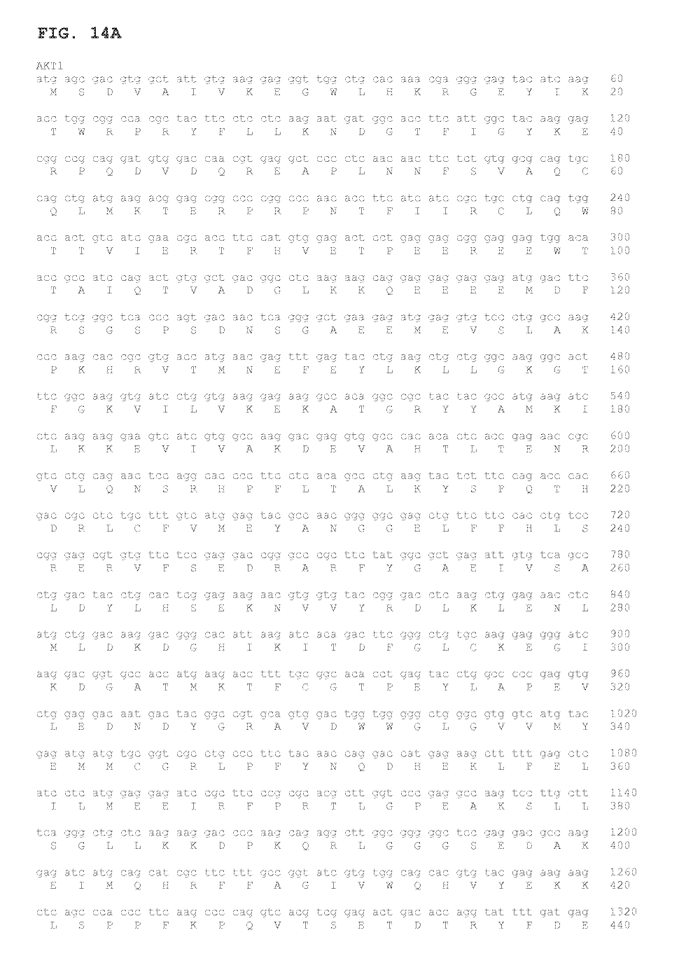

While mutational analysis of EGFR and KRAS is already widely viewed as the modern standard of care, the present assays uncovered additional events that also influenced clinical decisions. FIG. 12A illustrates the case of a breast cancer patient with metastatic disease that had progressed through all previous therapy regimens. Identification of the PIK3CA H1047L activating mutation in her tumor prompted enrollment in a clinical trial of a new PIK3CA inhibitor. FIG. 12B represents the case of a lung cancer patient with an activating mutation in EGFR that had previously responded to anti-EGFR therapy, but who recently relapsed. Re-biopsy and genotyping of the recurrence revealed the presence of the EGFR T790M mutation, which confers resistance to first-generation EGFR TKIs (Pao et al. (2005a) PLoS Med 2, e73). This finding prompted subsequent therapy with an irreversible EGFR TKI (Pfizer), which also targets the newly acquired T790M EGFR mutant (Riely, 2008). FIG. 12C is an example of how SNAPSHOT® genotyping can offer some insight into tumor heterogeneity. Here, profiling of bilateral tumor masses in a patient with lung cancer revealed two distinct genotypes. The results supported the clinical suspicion that this was not metastatic disease, but rather two synchronous early stage primary tumors. This interpretation provided a better prognosis for the patient, and affected the consideration for pursuing aggressive surgical therapy and adjuvant chemotherapy, directly impacting the management of her disease.

To further investigate sample heterogeneity within the primary tumors evaluated for clinical testing, all mutant cases were re-examined and the levels of mutant alleles identified by SNAPSHOT® genotyping were compared with the extent of stromal contamination in each original tumor specimen. As shown in Table 6, the extent of stromal contamination (column 2), and the levels of mutant alleles (column 3) are distinct for different tumor specimens, which is most likely reflective of an inability to accurately predict stromal contamination in a tridimensional tumor specimen, based on the histological evaluation of a single tumor section. In addition, some of these discrepancies may be due to tumor heterogeneity and the presence of activating mutations within variable subsets of tumor cell populations. Concerns with tumor heterogeneity underscore the importance of using highly sensitive mutation detection methods. This matter has been widely appreciated, particularly for mutations that confer resistance to targeted therapeutics where the detection of minor resistant clones, either in the primary tumor or during the course of treatment, is critical to predict response (Maheswaran et al. (2008) N Engl J Med 359, 366-377; Marchetti et al. (2009) Neoplasia 11, 1084-1092; Yung et al. (2009) Clin Cancer Res 15, 2076-2084). By contrast, the clinical implications of identifying low levels of drug-sensitizing mutations are currently unknown. To address this issue, the response of patients with low abundance EGFR sensitizing mutations to EGFR TKIs was examined. Within this small cohort, two patients (NA09-129 and NA09-184) were identified with low levels (<20%) of EGFR exon 19 deletions, both of whom achieved a clinical response to EGFR TKI therapy (Table 6). These results demonstrate that the use of targeted agents may be helpful even in cases where the sensitizing mutations are restricted to smaller clones of the tumor cell population. Importantly, these findings indicate that highly sensitive detection methods will be fundamental in identifying these patients.

### REFERENCES

Bamford et al. (2004) Br J Cancer 91, 355-358.
Berns et al. (2007) Cancer Cell 12, 395-402.
Blackford et al. (2009) Cancer Res 69, 3681-3688.
Chin and Gray (2008) Nature 452, 553-563.
Davies et al. (2005) Cancer Res 65, 7591-7595.
Di Nicolantonio et al. (2008) J Clin Oncol 26, 5705-5712.
Ding et al. (2008) Nature 455, 1069-1075.
Druker et al. (2001) N Engl JMed 344, 1031-1037.
Greenman et al. (2007) Nature 446, 153-158.
Hanahan and Weinberg (2000) Cell, 100, 57-70.
Hollestelle et al. (2007) Mol Cancer Res 5, 195-201.
Hughes et al. (2006) Blood 108, 28-37.
Jones et al. (2008) Science 321, 1801-1806.
Kobayashi et al. (2005) N Engl JMed 352, 786-792.
Ladanyi and Pao (2008) Mod Pathol 21 Suppl 2, S16-22.
Lievre et al. (2006) Cancer Res 66, 3992-3995.
Lindblad-Toh et al. (2000) Nat Genet 24, 381-386.
Lynch et al. (2004) N Engl JMed 350, 2129-2139.
Maheswaran et al. (2008) N Engl J Med 359, 366-377.
Marchetti et al. (2009) Neoplasia 11, 1084-1092.
McDermott et al. (2007) Proc Natl Acad Sci USA 104, 19936-19941.
Mok et al. (2009) N Engl J Med 361, 947-957.
Nakatani et al. (2002) Mod Pathol 15, 617-624.
Paez et al. (2004) Science 304, 1497-1500.
Pao et al. (2004) Proc Natl Acad Sci USA 101, 13306-13311.
Pao et al. (2005a) PLoS Med 2, e73.
Pao et al. (2005b) PLoS Med 2, e17.
Pedersen et al. (2001) Ann Oncol 12, 745-760.
Riely (2008) J Thorac Oncol 3, S146-149.
Rikova et al. (2007) Cell 131, 1190-1203.
Sartore-Bianchi et al. (2009) Cancer Res, 69, 1851-1857.
Sharma et al. (2007) Nat Rev Cancer 7, 169-181.
Sjoblom et al. (2006) Science 314, 268-274.
Slebos et al. (1991) J Natl Cancer Inst 83, 1024-1027.
Sordella et al. (2004) Science 305, 1163-1167.
Sos et al. (2009) J Clin Invest 119, 1727-1740.
Srinivasan et al. (2002) Am J Pathol 161, 1961-1971.
Stephens et al. (2005) Nat Genet, 37 590-592.
Subramanian and Govindan (2008) Lancet Oncol 9, 676-682.
Thomas et al. (2007) Nat Genet 39, 347-351.
Weinstein (2000) Carcinogenesis 21, 857-864.
Wood et al. (2007) Science 318, 1108-1113.
Yung et al. (2009) Clin Cancer Res 15, 2076-2084.
Zhu et al. (2008) Cancer Lett 265, 307-317.

TABLE 1

| | SNaPshot | | |
|---|---|---|---|
| Gene | coverage | Relevant drugs: launched (developer) | Relevant drugs: clinical testing phase¹ |
| APC | 15% | none | none |
| BRAF | 94% | Sorafenib (Bayer HealthCare Pharmaceuticals, | Raf inhibitors (4) |
| | | Onyx Pharmaceuticals) | MEK inhibitors (6) |
| | | | ERK inhibitor (1) |
| CTNNB1 | 74% | none | none |
| EGFR | 69% | Gefitinib (AstraZeneca) | 26 compounds |
| | | Cetuximab (ImClone Systems, Merck Serono, | |
| | | Bristol-Myers Squibb) | |
| | | Erlotinib hydrochloride (Genentech, OSI | |
| | | Pharmaceuticals, Roche) | |
| | | Panitumumab (Amgen) | |
| | | Nimotuzumab (YM BioSciences, Biotech | |
| | | Pharmaceuticals, Oncoscience, Daiichi | |
| | | Sankyo) | |
| | | Lapatinib (GlaxoSmithKline) | |
| FLT3 | 22% | Sorafenib (Bayer HealthCare Pharmaceuticals, | 12 compounds |
| | | Onyx Pharmaceuticals) | |
| | | Sunitinib (Pfizer) | |
| JAK2 | 99% | none | JAK2 inhibitors (5) |
| | | | STAT3 inhibitors (2) |
| KIT | 24% | Imatinib mesylate (Novartis Oncology) | 9 compounds |
| | | Sorafenib (Bayer HealthCare Pharmaceuticals, | |
| | | Onyx Pharmaceuticals) | |
| | | Sunitinib (Pfizer) | |
| KRAS | 98% | none | Raf inhibitors (4) |
| | | | MEK inhibitors (6) |
| | | | ERK inhibitor (1) |
| NOTCH1 | 9% | none | Notch1/Gamma-Secretase inhibitors (3) |
| NRAS | 97% | none | Raf inhibitors (4) |
| | | | MEK inhibitors (6) |
| | | | ERK inhibitor (1) |
| PIK3CA | 76% | mTOR inhibitors: | |
| | | Sirolinmus (Wyeth Pharmaceuticals) | PI3K inhibitors (9) |
| | | Everolimus (Novartis Pharmaceuticals) | PKB/AKT inhibitors (4) |
| | | Temsirolimus (Wyeth Pharmaceuticals) | mTOR inhibitors (7) |
| PTEN | 15% | mTOR inhibitors: | |
| | | Sirolinmus (Wyeth Pharmaceuticals) | PI3K inhibitors (9) |
| | | Everolimus (Novartis Pharmaceuticals) | PKB/AKT inhibitors (4) |
| | | Temsirolimus (WP) | mTOR inhibitors (7) |
| TP53 | 29% | none | none |

TABLE 2

| Tumor type | Total no. of cases | Mutations (no. of cases) |
|---|---|---|
| Breast | 33 | KRAS G12V + PIK3CA E545K (1)* |
| | | PIK3CA H1047L (1) |
| | | PIK3CA H1047R (2) |
| | | TP53 R175H (1) |
| | | TP53 R248Q (1) |
| Chronic Myeloproliferative Disorder | 10 | JAK2 V617F (4) |
| Colorectal | 30 | APC R1114X (1) |
| | | BRAF V600E (1) |
| | | KRAS G12C (1) |
| | | KRAS G12D (2) |
| | | KRAS G12S (1) |
| | | KRAS G12V (2) |
| | | KRAS G12V + PIK3CA E545K (1) |
| | | KRAS G13D (1) |
| | | KRAS G13D + PIK3CA R88Q (1)* |
| | | KRAS G13D + TP53 R273H (1)* |
| | | NRAS G12D (2)* |
| | | NRAS Q61H + TP53 R175H (1)* |
| | | PI3KCA E545K (1) |
| | | TP53 R175H (1) |
| Lung | 87 | CTNNB1 S37F + EGFR E746_A750del (1)* |
| | | EGFR E746_A750del (6) |
| | | EGFR E746_A750del + EGFR T790M + TP53 R175H (1)* |
| | | EGFR L858R (4) |
| | | EGFR L858R + EGFR T790M (1) |
| | | KRAS G12A (2) |
| | | KRAS G12C (10) |
| | | KRAS G12D (1) |
| | | KRAS G12D + TP53 R248Q (1)* |
| | | KRAS G12V (3) |
| | | KRAS G13D (1) |
| | | NRAS Q61L + TP53 R248P (1)* |
| | | PIK3CA E542K (1) |
| | | TP53 R248Q (1) |
| | | TP53 R273L (1) |
| Melanoma | 11 | BRAF V600E (4) |
| | | BRAF V600M (1) |
| | | NRAS Q61L (1) |
| | | NRAS Q61R (1) |
| Pancreatic | 23 | KRAS G12D (2) |
| | | KRAS G12D + TP53 R175H (1)* |
| | | KRAS G12R (2) |
| | | KRAS G12V (5) |
| | | KRAS G12V + TP53 R248Q (1)* |
| Prostate | 20 | CTNNB1 S33C (1) |
| | | CTNNB1 S37Y + PIK3CA E542K (1)* |
| | | KRAS G13R (1)* |
| Other | 36 | BRAF V600E (1)*, unknown primary, presumed breast |
| | | KRAS G12D (1), cervical |
| | | TP53 R306X (1)*, thyroid Hurthle cell carcinoma |

*Mutations or combination of mutations that are rare or not-previously described in the corresponding tumor type.

TABLE 3A

| | NUCLEOTIDE POSITION |
|---|---|
| | TESTED BY GENOTYPING |
| GENE_SYMBOL | ASSAY |
| AKT1 | c.49G |
| APC | c.3340C |
| APC | c.4012C |
| APC | c.4348C |
| APC | c.4666_4667insA |
| BRAF | c.1397G |
| BRAF | c.1406G |
| BRAF | c.1789C |
| BRAF | c.1798G |
| BRAF | c.1799T |
| CTNNB1 | c.101G |
| CTNNB1 | c.109T |
| CTNNB1 | c.110C |
| CTNNB1 | c.121A |
| CTNNB1 | c.122C |
| CTNNB1 | c.133T |
| CTNNB1 | c.134C |
| CTNNB1 | c.94G |
| CTNNB1 | c.95A |
| CTNNB1 | c.98C |
| EGFR | c.2155G |
| EGFR | c.2156G |
| EGFR | c.2235_2249del15 F |
| EGFR | c.2235_2249del15 R |
| EGFR | c.2236_2250del15 F |
| EGFR | c.2236_2250del15 R |
| EGFR | c.2369C |
| EGFR | c.2573T |
| EGFR | c.2582T |
| FLT3 | c.2503G |
| IDH1 | c.394C |
| IDH1 | c.395G |
| JAK2 | c.1849G |
| KIT | c.2447A |
| KRAS | c.181C |
| KRAS | c.182A |
| KRAS | c.183A |
| KRAS | c.34G |
| KRAS | c.35G |
| KRAS | c.37G |
| KRAS | c.38G |
| MAP2K1 | c.167A |
| MAP2K1 | c.171G |
| MAP2K1 | c.199G |
| NOTCH1 | c.4724T |
| NOTCH1 | c.4802T |
| NRAS | c.181C |
| NRAS | c.182A |
| NRAS | c.183A |
| NRAS | c.34G |
| NRAS | c.35G |
| NRAS | c.37G |
| NRAS | c.38G |
| PIK3CA | c.1624G |
| PIK3CA | c.1633G |
| PIK3CA | c.1636C |
| PIK3CA | c.1637A |
| PIK3CA | c.263G |
| PIK3CA | c.3139C |
| PIK3CA | c.3140A |
| PIK3CA | c.3145G |
| PTEN | c.388C |
| PTEN | c.517C |
| PTEN | c.697C |
| PTEN | c.800delA |
| TP53 | c.524G |
| TP53 | c.733G |
| TP53 | c.742C |
| TP53 | c.743G |
| TP53 | c.817C |
| TP53 | c.818G |
| TP53 | c.916C |

TABLE 3B

| GENE_SYMBOL | GENE_ID | AA_MUTATION | CDS_MUTATION | MUT_ID | MUT_COUNT |
|---|---|---|---|---|---|
| AKT1 | 207 | E17K | 49G > A | | |
| APC | 324 | R1114X | 3340C > T | 13125 | 19 |
| APC | 324 | Q1338X | 4012C > T | 13129 | 21 |
| APC | 324 | R1450X | 4348C > T | 13127 | 100 |
| APC | 324 | T1556fs*3 | 4660_4661insA | 19695 | 35 |
| APC | 324 | T1556fs*3 | 4662_4663insA | 18734 | 13 |
| APC | 324 | T1556fs*3 | 4665_4666insA | 19020 | 9 |
| APC | 324 | T1556fs*3 | 4666_4667insA | 18561 | 70 |
| BRAF | 673 | V600A | 1799T > C | 18443 | 22 |
| BRAF | 673 | V600E | 1799T > A | 476 | 7762 |
| BRAF | 673 | V600G | 1799T > G | 6137 | 1 |
| BRAF | 673 | V600M | 1798G > A | 1130 | 25 |
| BRAF | 673 | G466E | 1397G > A | | |
| BRAF | 673 | G466A | 1397G > C | | |
| BRAF | 673 | G466V | 1397G > T | | |
| BRAF | 673 | G469E | 1406G > A | | |
| BRAF | 673 | G469A | 1406G > C | | |
| BRAF | 673 | G469V | 1406G > T | | |
| BRAF | 673 | L597V | 1789C > G | | |
| CTNNB1 | 1499 | D32A | 95A > C | 5690 | 11 |
| CTNNB1 | 1499 | D32G | 95A > G | 5681 | 47 |
| CTNNB1 | 1499 | D32H | 94G > C | 5668 | 31 |
| CTNNB1 | 1499 | D32N | 94G > A | 5672 | 47 |
| CTNNB1 | 1499 | D32V | 95A > T | 5691 | 16 |
| CTNNB1 | 1499 | D32Y | 94G > T | 5661 | 95 |
| CTNNB1 | 1499 | S33C | 98C > G | 5677 | 115 |
| CTNNB1 | 1499 | S33F | 98C > T | 5669 | 67 |
| CTNNB1 | 1499 | S33Y | 98C > A | 5673 | 43 |
| CTNNB1 | 1499 | G34E | 101G > A | 5671 | 57 |
| CTNNB1 | 1499 | G34V | 101G > T | 5670 | 60 |
| CTNNB1 | 1499 | S37A | 109T > G | 5675 | 58 |
| CTNNB1 | 1499 | S37C | 110C > G | 5679 | 114 |
| CTNNB1 | 1499 | S37F | 110C > T | 5662 | 135 |
| CTNNB1 | 1499 | S37P | 109T > C | 5687 | 12 |
| CTNNB1 | 1499 | S37T | 109T > A | 5729 | 1 |
| CTNNB1 | 1499 | S37Y | 110C > A | 5666 | 20 |
| CTNNB1 | 1499 | T41A | 121A > G | 5664 | 315 |
| CTNNB1 | 1499 | T41I | 122C > T | 5676 | 61 |
| CTNNB1 | 1499 | T41N | 122C > A | 5730 | 3 |
| CTNNB1 | 1499 | T41P | 121A > C | 5688 | 3 |
| CTNNB1 | 1499 | T41S | 122C > G | 5701 | 2 |
| CTNNB1 | 1499 | T41S | 121A > T | 5716 | 3 |
| CTNNB1 | 1499 | S45A | 133T > G | 5685 | 7 |
| CTNNB1 | 1499 | S45C | 134C > G | 5689 | 15 |
| CTNNB1 | 1499 | S45F | 134C > T | 5667 | 239 |
| CTNNB1 | 1499 | S45P | 133T > C | 5663 | 104 |
| CTNNB1 | 1499 | S45T | 133T > A | 5719 | 1 |
| CTNNB1 | 1499 | S45Y | 134C > A | 5692 | 13 |
| EGFR | 1956 | G719C | 2155G > T | 6253 | 16 |
| EGFR | 1956 | G719S | 2155G > A | 6252 | 21 |
| EGFR | 1956 | E746_A750del | 2235_2249del15 | 6223 | 633 |
| EGFR | 1956 | E746_A750del | 2236_2250del15 | 6225 | 398 |
| EGFR | 1956 | T790M | 2369C > T | 6240 | 81 |
| EGFR | 1956 | L858Q | 2573T > A | 29578 | 3 |
| EGFR | 1956 | L858R | 2573T > G | 6224 | 1683 |
| EGFR | 1956 | G719D | 2156G > A | | |
| EGFR | 1956 | G719A | 2156G > C | | |
| EGFR | 1956 | L861Q | 2582T > A | | |
| EGFR | 1956 | L861R | 2582T > G | | |
| FLT3 | 2322 | D835H | 2503G > C | 785 | 28 |
| FLT3 | 2322 | D835N | 2503G > A | 789 | 6 |
| FLT3 | 2322 | D835Y | 2503G > T | 783 | 163 |
| IDH1 | 3417 | R132S | 394C > A | | |
| IDH1 | 3417 | R132G | 394C > G | | |
| IDH1 | 3417 | R132C | 394C > T | | |
| IDH1 | 3417 | R132H | 395G > A | | |
| IDH1 | 3417 | R132L | 395G > T | | |
| JAK2 | 3717 | V617F | 1849G > T | 12600 | 14240 |
| KIT | 3815 | D816A | 2447A > C | 24675 | 2 |
| KIT | 3815 | D816G | 2447A > G | 12711 | 2 |
| KIT | 3815 | D816V | 2447A > T | 1314 | 670 |
| KRAS | 3845 | G12A | 35G > C | 522 | 697 |
| KRAS | 3845 | G12C | 34G > T | 516 | 1628 |
| KRAS | 3845 | G12D | 35G > A | 521 | 4473 |
| KRAS | 3845 | G12R | 34G > C | 518 | 528 |
| KRAS | 3845 | G12S | 34G > A | 517 | 745 |
| KRAS | 3845 | G12V | 35G > T | 520 | 2989 |
| KRAS | 3845 | G13A | 38G > C | 533 | 21 |
| KRAS | 3845 | G13C | 37G > T | 527 | 118 |
| KRAS | 3845 | G13D | 38G > A | 532 | 1192 |
| KRAS | 3845 | G13R | 37G > C | 529 | 24 |
| KRAS | 3845 | G13S | 37G > A | 528 | 46 |
| KRAS | 3845 | G13V | 38G > T | 534 | 17 |
| KRAS | 3845 | Q61K | 181C > A | | |
| KRAS | 3845 | Q61E | 181C > G | | |
| KRAS | 3845 | Q61P | 182A > C | | |
| KRAS | 3845 | Q61R | 182A > G | | |
| KRAS | 3845 | Q61L | 182A > T | | |
| KRAS | 3845 | Q61H | 183A > C | | |
| KRAS | 3845 | Q61H | 183A > T | | |
| MAP2K1 | 5604 | Q56P | 167A > C | | |
| MAP2K1 | 5604 | K57N | 171G > T | | |
| MAP2K1 | 5604 | D67N | 199G > A | | |
| NOTCH1 | 4851 | L1575P | 4724T > C | 12772 | 12 |
| NOTCH1 | 4851 | L1601P | 4802T > C | 12771 | 18 |
| NRAS | 4893 | G12A | 35G > C | 565 | 33 |
| NRAS | 4893 | G12C | 34G > T | 562 | 56 |
| NRAS | 4893 | G12D | 35G > A | 564 | 283 |
| NRAS | 4893 | G12R | 34G > C | 561 | 14 |
| NRAS | 4893 | G12S | 34G > A | 563 | 102 |
| NRAS | 4893 | G12V | 35G > T | 566 | 46 |
| NRAS | 4893 | G13A | 38G > C | 575 | 16 |
| NRAS | 4893 | G13C | 37G > T | 570 | 20 |
| NRAS | 4893 | G13D | 38G > A | 573 | 147 |
| NRAS | 4893 | G13R | 37G > C | 569 | 55 |
| NRAS | 4893 | G13S | 37G > A | 571 | 4 |
| NRAS | 4893 | G13V | 38G > T | 574 | 50 |
| NRAS | 4893 | Q61E | 181C > G | 581 | 9 |
| NRAS | 4893 | Q61H | 183A > T | 585 | 51 |
| NRAS | 4893 | Q61H | 183A > C | 586 | 29 |
| NRAS | 4893 | Q61K | 181C > A | 580 | 381 |
| NRAS | 4893 | Q61L | 182A > T | 583 | 111 |
| NRAS | 4893 | Q61P | 182A > C | 582 | 19 |
| NRAS | 4893 | Q61Q | 183A > G | 587 | 3 |
| NRAS | 4893 | Q61R | 182A > G | 584 | 506 |
| PIK3CA | 5290 | R88Q | 263G > A | 746 | 15 |
| PIK3CA | 5290 | E542K | 1624G > A | 760 | 218 |
| PIK3CA | 5290 | E542Q | 1624G > C | 17442 | 4 |
| PIK3CA | 5290 | E545K | 1633G > A | 763 | 381 |
| PIK3CA | 5290 | E545Q | 1633G > C | 27133 | 5 |
| PIK3CA | 5290 | Q546E | 1636C > G | 6147 | 8 |
| PIK3CA | 5290 | Q546K | 1636C > A | 766 | 28 |
| PIK3CA | 5290 | Q546L | 1637A > T | 25041 | 4 |
| PIK3CA | 5290 | Q546P | 1637A > C | 767 | 4 |
| PIK3CA | 5290 | Q546R | 1637A > G | 12459 | 7 |
| PIK3CA | 5290 | H1047L | 3140A > T | 776 | 71 |
| PIK3CA | 5290 | H1047R | 3140A > G | 775 | 560 |
| PIK3CA | 5290 | H1047Y | 3139C > T | 774 | 21 |
| PIK3CA | 5290 | G1049R | 3145G > C | 12597 | 10 |
| PIK3CA | 5290 | G1049S | 3145G > A | 777 | 6 |
| PTEN | 5728 | R130X | 388C > T | 5152 | 48 |
| PTEN | 5728 | R130G | 388C > G | 5219 | 49 |
| PTEN | 5728 | R130R | 388C > A | 5329 | 1 |
| PTEN | 5728 | R173C | 517C > T | 5089 | 26 |
| PTEN | 5728 | R233X | 697C > T | 5154 | 51 |
| PTEN | 5728 | R233R | 697C > A | 13457 | 1 |
| PTEN | 5728 | K267fs*9 | 800delA | 5809 | 40 |
| PTEN | 5728 | K267fs*9 | 799delA | 5862 | 2 |
| TP53 | 7157 | R175H | 524G > A | 10648 | 22 |
| TP53 | 7157 | R175L | 524G > T | 10718 | 2 |
| TP53 | 7157 | G245C | 733G > T | 11081 | 3 |
| TP53 | 7157 | G245R | 733G > C | 10957 | 1 |
| TP53 | 7157 | G245S | 733G > A | 6932 | 12 |
| TP53 | 7157 | R248G | 742C > G | 11564 | 1 |
| TP53 | 7157 | R248L | 743G > T | 6549 | 4 |
| TP53 | 7157 | R248P | 743G > C | 11491 | 1 |
| TP53 | 7157 | R248Q | 743G > A | 10662 | 31 |
| TP53 | 7157 | R248W | 742C > T | 10656 | 16 |
| TP53 | 7157 | R273C | 817C > T | 10659 | 19 |
| TP53 | 7157 | R273H | 818G > A | 10660 | 26 |
| TP53 | 7157 | R273L | 818G > T | 10779 | 6 |
| TP53 | 7157 | R306X | 916C > T | 10663 | 6 |

| GENE_SYMBOL | MUT_FREQUENCY | VALIDATION_CONTROL |
|---|---|---|
| AKT1 | | |
| APC | 1.08% | cell line |
| | | (LoVo) |
| APC | 1.20% | cell line |
| | | (SW620) |
| APC | 5.70% | oligonucleotide |
| | | (S. ctrl_APC4348C > |
| | | T) |
| APC | 2.00% | oligonucleotide |
| | | (A. ctrl_APC4666_67insA) |
| APC | 0.74% | oligonucleotide |
| | | (A. ctrl_APC4666_67insA) |
| APC | 0.51% | oligonucleotide |
| | | (A. ctrl_APC4666_67insA) |
| APC | 3.99% | oligonucleotide |
| | | (A. ctrl_APC4666_67insA) |
| BRAF | 0.26% | none |
| BRAF | 93.27% | primary |
| | | tumor |
| | | (FFPE_NA08- |
| | | 249) |
| BRAF | 0.01% | none |
| BRAF | 0.30% | oligonucleotide |
| | | (A. ctrl_BRAF1798G > |
| | | A) |
| BRAF | | |
| BRAF | | |
| BRAF | | |
| BRAF | | |
| BRAF | | |
| BRAF | | |
| BRAF | | |
| CTNNB1 | 0.48% | none |
| CTNNB1 | 2.05% | oligonucleotide |
| | | (A. ctrl_CTNNB1_98C > |
| | | G) |
| CTNNB1 | 1.35% | oligonucleotide |
| | | (A. ctrl_CTNNB1_94G > |
| | | C) |
| CTNNB1 | 2.05% | oligonucleotide |
| | | (A. ctrl_CTNNB1_94G > |
| | | A) |
| CTNNB1 | 0.70% | none |
| CTNNB1 | 4.14% | oligonucleotide |
| | | (A. ctrl_CTNNB1_94G > |
| | | T) |
| CTNNB1 | 5.01% | oligonucleotide |
| | | (A. ctrl_CTNNB1_98C > |
| | | G) |
| CTNNB1 | 2.92% | cell line |
| | | (SW1573) |
| CTNNB1 | 1.87% | cell line |
| | | (SW48) |
| CTNNB1 | 2.48% | oligonucleotide |
| | | (A. ctrl_CTNNB1_101G > |
| | | A) |
| CTNNB1 | 2.61% | oligonucleotide |
| | | (A. ctrl_CTNNB1_101G > |
| | | T) |
| CTNNB1 | 2.53% | oligonucleotide |
| | | (A. ctrl_CTNNB1_109T > |
| | | G) |
| CTNNB1 | 4.96% | oligonucleotide |
| | | (A. ctrl_CTNNB1_110C > |
| | | G) |
| CTNNB1 | 5.88% | oligonucleotide |
| | | (A. ctrl_CTNNB1_110C > |
| | | T) |
| CTNNB1 | 0.52% | none |
| CTNNB1 | 0.04% | none |
| CTNNB1 | 0.87% | oligonucleotide |
| | | (A. ctrl_CTNNB1_110C > |
| | | A) |
| CTNNB1 | 13.71% | cell line |
| | | (A-427) |
| CTNNB1 | 2.66% | oligonucleotide |
| | | (S. ctrl_CTNNB1_122C > |
| | | T) |
| CTNNB1 | 0.13% | none |
| CTNNB1 | 0.13% | none |
| CTNNB1 | 0.09% | none |
| CTNNB1 | 0.13% | none |
| CTNNB1 | 0.30% | none |
| CTNNB1 | 0.65% | none |
| CTNNB1 | 10.40% | cell line |
| | | (LS174T) |
| CTNNB1 | 4.53% | oligonucleotide |
| | | (S. ctrl_CTNNB1_133T > |
| | | C) |
| CTNNB1 | 0.04% | none |
| CTNNB1 | 0.57% | none |
| EGFR | 0.39% | oligonucleotide |
| | | (A. ctrl_EGFR2155G > |
| | | T) |
| EGFR | 0.51% | cell line |
| | | (SW48) |
| EGFR | 15.45% | cell line |
| | | (PC9) |
| EGFR | 9.71% | primary |
| | | tumor |
| | | (FFPE_NA08- |
| | | 0247) |
| EGFR | 1.98% | cell line |
| | | (NCI- |
| | | H1975) |
| EGFR | 0.07% | none |
| EGFR | 41.07% | cell line |
| | | (NCI- |
| | | H1975) |
| EGFR | | |
| EGFR | | |
| EGFR | | |
| EGFR | | |
| FLT3 | 3.10% | none |
| FLT3 | 0.67% | none |
| FLT3 | 18.07% | cell line |
| | | (MO-4)* |
| IDH1 | | |
| IDH1 | | |
| IDH1 | | |
| IDH1 | | |
| IDH1 | | |
| JAK2 | 98.68% | primary |
| | | tumor |
| | | (blood |
| | | DNA_NA08- |
| | | 0257) |
| KIT | 0.07% | none |
| KIT | 0.07% | none |
| KIT | 23.49% | oligonucleotide |
| | | (A. ctrl_KIT2447A > |
| | | T) |
| KRAS | 5.45% | oligonucleotide |
| | | (A. ctrl_KRAS35G > C) |
| KRAS | 12.74% | cell line |
| | | (MOLT-4) |
| KRAS | 35.00% | cell line |
| | | (A427) |
| KRAS | 4.13% | cell line |
| | | (Cal-62) |
| KRAS | 5.83% | cell line |
| | | (A549) |
| KRAS | 23.39% | cell line |
| | | (LCLC97TMI) |
| KRAS | 0.16% | none |
| KRAS | 0.92% | oligonucleotide |
| | | (A. ctrl_KRAS37G > |
| | | T) |
| KRAS | 9.33% | cell line |
| | | (LoVo) |
| KRAS | 0.19% | cell line |
| | | (K052) |
| KRAS | 0.36% | none |
| KRAS | 0.13% | none |
| KRAS | | |
| KRAS | | |
| KRAS | | |
| KRAS | | |
| KRAS | | |
| KRAS | | |
| KRAS | | |
| MAP2K1 | | |
| MAP2K1 | | |
| MAP2K1 | | |
| NOTCH1 | 3.70% | oligonucleotide |
| | | (S. ctrl_NOTCH1_4724T > |
| | | C) |
| NOTCH1 | 5.56% | oligonucleotide |
| | | (A. ctrl_NOTCH1_4802T > |
| | | C) |
| NRAS | 1.66% | oligonucleotide |
| | | (S. ctrl_NRAS35G > C) |
| NRAS | 2.82% | cell line |
| | | (MOLT-4) |
| NRAS | 14.25% | cell line |
| | | (PA-1) |
| NRAS | 0.70% | none |
| NRAS | 5.14% | oligonucleotide |
| | | (S. ctrl_NRAS34G > |
| | | A) |
| NRAS | 2.32% | cell line |
| | | (GA-10) |
| NRAS | 0.81% | none |
| NRAS | 1.01% | oligonucleotide |
| | | (S. ctrl_NRAS37G > |
| | | T) |
| NRAS | 7.40% | oligonucleotide |
| | | (S. ctrl_NRAS38G > |
| | | A) |
| NRAS | 2.77% | cell line |
| | | (K052) |
| NRAS | 0.20% | none |
| NRAS | 2.52% | oligonucleotide |
| | | (S. ctrl_NRAS38G > |
| | | T) |
| NRAS | 0.45% | none |
| NRAS | 2.57% | oligonucleotide |
| | | (S. ctrl_NRAS183A > |
| | | T) |
| NRAS | 1.46% | oligonucleotide |
| | | (S. ctrl_NRAS183A > |
| | | C) |
| NRAS | 19.18% | cell line |
| | | (HMV-11) |
| NRAS | 5.59% | cell line |
| | | (BFTC- |
| | | 905) |
| NRAS | 0.96% | oligonucleotide |
| | | (A. ctrl_NRAS182A > |
| | | C) |
| NRAS | 0.15% | none |
| NRAS | 25.48% | oligonucleotide |
| | | (A. ctrl_NRAS182A > |
| | | G) |
| PIK3CA | 0.85% | cell line |
| | | (SNG-M) |
| PIK3CA | 12.36% | cell line |
| | | (Cal51) |
| PIK3CA | 0.23% | none |
| PIK3CA | 21.60% | cell line |
| | | (BFTC- |
| | | 909) |
| PIK3CA | 0.28% | none |
| PIK3CA | 0.45% | none |
| PIK3CA | 1.59% | oligonucleotide |
| | | (A. ctrl_PIK3CA1636C > |
| | | A) |
| PIK3CA | 0.23% | none |
| PIK3CA | 0.23% | none |
| PIK3CA | 0.40% | cell line |
| | | (22RVI) |
| PIK3CA | 4.02% | oligonucleotide |
| | | (S. ctrl_PIK3CA3140A > |
| | | T) |
| PIK3CA | 31.75% | cell line |
| | | (LS174T) |
| PIK3CA | 1.19% | cell line |
| | | (MFE-280) |
| PIK3CA | 0.57% | cell line |
| | | (HEC-1) |
| PIK3CA | 0.34% | oligonucleotide |
| | | (S. ctrl_PIK3CA3145G > |
| | | A) |
| PTEN | 3.22% | oligonucleotide |
| | | (A. ctrl_PTEN388C > |
| | | T) |
| PTEN | 3.28% | oligonucleotide |
| | | (A. ctrl_PTEN388C > |
| | | G) |
| PTEN | 0.07% | none |
| PTEN | 1.74% | cell line |
| | | (639V) |
| PTEN | 3.42% | cell line |
| | | (SF295) |
| PTEN | 0.07% | none |
| PTEN | 2.68% | cell line |
| | | (MOLT-4) |
| PTEN | 0.13% | cell line |
| | | (MOLT-4) |
| TP53 | 4.27% | cell line |
| | | (VM- |
| | | CUB1) |
| TP53 | 0.39% | none |
| TP53 | 0.58% | none |
| TP53 | 0.19% | none |
| TP53 | 2.33% | oligonucleotide |
| | | (S. ctrl_TP53_733G > |
| | | A) |
| TP53 | 0.19% | none |
| TP53 | 0.78% | none |
| TP53 | 0.19% | none |
| TP53 | 6.02% | cell line |
| | | (639V) |
| TP53 | 3.11% | cell line |
| | | (Colo680N) |
| TP53 | 3.69% | oligonucleotide |
| | | (A. ctrl_TP53_817C > |
| | | T) |
| TP53 | 5.05% | cell line |
| | | (NCI- |
| | | H1975) |
| TP53 | 1.17% | cell line |
| | | (HCC38) |
| TP53 | 1.17% | cell line |
| | | (MOLT-4) |

TABLE 4
Primary cancer samples and tumor genotyping data

| | | | | | | | | | | | EGFR EXON 19 |
|---|---|---|---|---|---|---|---|---|---|---|---|
| | | | | | | | | | | MUTATIONS | STATUS |
| SAMPLE_ID | TUMOR_TYPE | SEX | AGE | STAGE | SMOKING STATUS | PACKS_PER_YEAR | IHC_DATA | SAMPLE_TYPE | RESULTS | (SNAP-SHOT) | (SIZING ASSAY) |
| NA09- | ADENOCARCINOMA OF | F | 60 | IV | N/A | N/A | ER(−)/ | Research | Mutation | BRAF | N/A |
| 004 | UNKNOWN PRIMARY, | | | | | | PR(−)/ | | | V600E | |
| | PRESUMED BREAST | | | | | | Her-2(−) | | | (1799T > A) | |
| NA09- | BLADDER, SMALL CELL | M | 60 | N/A | N/A | N/A | N/A | Research | Normal | No | N/A |
| 130 | NEUROENDOCRINE | | | | | | | | | Mutation | |
| | CARCINOMA | | | | | | | | | | |
| NA09- | BRAIN, GLIOBLASTOMA | M | 55 | N/A | N/A | N/A | N/A | Research | Normal | No | N/A |
| 102 | | | | | | | | | | Mutation | |
| NA09- | BREAST, DUCTAL | F | 48 | IA | N/A | N/A | ER(+)/ | Research | Mutation | PIK3CA | N/A |
| 518 | CARCINOMA | | | | | | PR(N/A)/ | | | H1047L | |
| | | | | | | | Her-2(−) | | | (3140A > T) | |
| NA08- | BREAST, DUCTAL | F | 49 | N/A | N/A | N/A | N/A | Research | Mutation | TP53 | N/A |
| 066 | CARCINOMA | | | | | | | | | R175H | |
| | | | | | | | | | | (524G > A) | |
| NA08- | BREAST, DUCTAL | F | 69 | IV | N/A | N/A | ER(−)/ | Research | Normal | No | N/A |
| 201 | CARCINOMA | | | | | | PR(−)/ | | | Mutation | |
| | | | | | | | Her-2(−) | | | | |
| NA08- | BREAST, DUCTAL | M | 56 | IV | N/A | N/A | ER(+)/ | Research | Normal | No | N/A |
| 179 | CARCINOMA | | | | | | PR(+)/ | | | Mutation | |
| | | | | | | | Her-2(−) | | | | |
| NA08- | BREAST, DUCTAL | M | 76 | II | N/A | N/A | ER(+)/ | Research | Normal | No | N/A |
| 200 | CARCINOMA | | | | | | PR(+)/ | | | Mutation | |
| | | | | | | | Her-2(−) | | | | |
| NA08- | BREAST, DUCTAL | F | 73 | II | N/A | N/A | ER(−)/ | Research | Normal | No | N/A |
| 176 | CARCINOMA | | | | | | PR(−)/ | | | Mutation | |
| | | | | | | | Her-2(−) | | | | |
| NA08- | BREAST, DUCTAL | F | 41 | II | N/A | N/A | ER(+)/ | Research | Normal | No | N/A |
| 187 | CARCINOMA | | | | | | PR(+)/ | | | Mutation | |
| | | | | | | | Her-2(−) | | | | |
| NA08- | BREAST, DUCTAL | F | 47 | IV | N/A | N/A | ER(+)/ | Research | Normal | No | N/A |
| 190 | CARCINOMA | | | | | | PR(−)/ | | | Mutation | |
| | | | | | | | Her-2(−) | | | | |
| NA08- | BREAST, DUCTAL | F | 43 | III | N/A | N/A | ER(+)/ | Research | Normal | No | N/A |
| 183 | CARCINOMA | | | | | | PR(+)/ | | | Mutation | |
| | | | | | | | Her-2(−) | | | | |
| NA08- | BREAST, DUCTAL | F | 57 | II | N/A | N/A | ER(faint)/ | Research | Normal | No | N/A |
| 185 | CARCINOMA | | | | | | PR(−)/ | | | Mutation | |
| | | | | | | | Her-2(−) | | | | |
| NA08- | BREAST, DUCTAL | F | 50 | I | N/A | N/A | ER(−)/ | Research | Normal | No | N/A |
| 186 | CARCINOMA | | | | | | PR(−)/ | | | Mutation | |
| | | | | | | | Her-2(−) | | | | |
| NA08- | BREAST, DUCTAL | F | 50 | III | N/A | N/A | ER(+)/ | Research | Normal | No | N/A |
| 188 | CARCINOMA | | | | | | PR(+)/ | | | Mutation | |
| | | | | | | | Her-2(−) | | | | |
| NA08- | BREAST, DUCTAL | F | 32 | II | N/A | N/A | ER(+)/ | Research | Normal | No | N/A |
| 182 | CARCINOMA | | | | | | PR(+)/ | | | Mutation | |
| | | | | | | | Her-2(−) | | | | |
| NA08- | BREAST, DUCTAL | F | 38 | I | N/A | N/A | ER(+)/ | Research | Normal | No | N/A |
| 181 | CARCINOMA | | | | | | PR(+)/ | | | Mutation | |
| | | | | | | | Her-2(−) | | | | |
| NA08- | BREAST, DUCTAL | F | 49 | IV | N/A | N/A | ER(+)/ | Research | Mutation | PIK3CA | N/A |
| 189 | CARCINOMA | | | | | | PR(+)/ | | | H1047R | |
| | | | | | | | Her-2(−) | | | (3140A > G) | |
| NA08- | BREAST, DUCTAL | F | 45 | I | N/A | N/A | ER(+)/ | Research | Normal | No | N/A |
| 205 | CARCINOMA | | | | | | PR(+)/ | | | Mutation | |
| | | | | | | | Her-2(+) | | | | |
| NA08- | BREAST, DUCTAL | F | 76 | IV | N/A | N/A | ER(+)/ | Research | Normal | No | N/A |
| 211 | CARCINOMA | | | | | | PR(+)/ | | | mutation | |
| | | | | | | | Her-2(N/A) | | | | |
| NA08- | BREAST, DUCTAL | F | 44 | II | N/A | N/A | ER(+)/ | Research | Normal | No | N/A |
| 214 | CARCINOMA | | | | | | PR(+)/ | | | Mutation | |
| | | | | | | | Her-2(−) | | | | |
| NA08- | BREAST, DUCTAL | F | 64 | II | N/A | N/A | ER(+)/ | Research | Normal | No | N/A |
| 206 | CARCINOMA | | | | | | PR(−)/ | | | Mutation | |
| | | | | | | | Her-2(+) | | | | |
| NA08- | BREAST, DUCTAL | F | 54 | IV | N/A | N/A | ER(−)/ | Research | Normal | No | N/A |
| 215 | CARCINOMA | | | | | | PR(−)/ | | | Mutation | |
| | | | | | | | Her-2(−) | | | | |
| NA08- | BREAST, DUCTAL | F | 38 | II | N/A | N/A | ER(+)/ | Research | Normal | No | N/A |
| 197 | CARCINOMA | | | | | | PR(+)/ | | | Mutation | |
| | | | | | | | Her-2(+) | | | | |
| NA08- | BREAST, DUCTAL | F | 64 | IV | N/A | N/A | ER(+)/ | Research | Normal | No | N/A |
| 210 | CARCINOMA | | | | | | PR(+)/ | | | Mutation | |
| | | | | | | | Her-2(−) | | | | |
| NA08- | BREAST, DUCTAL | F | 49 | IV | N/A | N/A | ER(+)/ | Research | Normal | No | N/A |
| 207 | CARCINOMA | | | | | | PR(+)/ | | | Mutation | |
| | | | | | | | Her-2(−) | | | | |
| NA08- | BREAST, DUCTAL | F | 39 | IV | N/A | N/A | ER(−)/ | Research | Normal | No | N/A |
| 202 | CARCINOMA | | | | | | PR(−)/ | | | Mutation | |
| | | | | | | | Her-2(+) | | | | |
| NA09- | BREAST, DUCTAL | F | 54 | IV | N/A | N/A | ER(−)/ | Research | Normal | No | N/A |
| 065 | CARCINOMA | | | | | | PR(−)/ | | | Mutation | |
| | | | | | | | Her-2(−) | | | | |
| NA09- | BREAST, DUCTAL | F | 72 | III | N/A | N/A | ER(−)/ | Research | Normal | No | N/A |
| 119 | CARCINOMA | | | | | | PR(−)/ | | | Mutation | |
| | | | | | | | Her-2(−) | | | | |
| NA09- | BREAST, DUCTAL | F | 30 | IV | N/A | N/A | ER(−)/ | Research | Mutation | TP53 | N/A |
| 133 | CARCINOMA | | | | | | PR(−)/ | | | R248Q | |
| | | | | | | | Her-2(−) | | | (743G > A) | |
| NA09- | BREAST, DUCTAL | F | 53 | II | N/A | N/A | ER(−)/ | Research | Normal | No | N/A |
| 124 | CARCINOMA | | | | | | PR(−)/ | | | Mutation | |
| | | | | | | | Her-2(−) | | | | |
| NA09- | BREAST, DUCTAL | F | 46 | II | N/A | N/A | ER(+)/ | Research | Normal | No | N/A |
| 266 | CARCINOMA | | | | | | PR(+)/ | | | Mutation | |
| | | | | | | | Her-2(−) | | | | |
| NA08- | BREAST, LOBULAR | F | 58 | IV | N/A | N/A | ER(+)/ | Research | Normal | No | N/A |
| 054 | CARCINOMA | | | | | | PR(+)/ | | | Mutation | |
| | | | | | | | Her-2(−) | | | | |
| NA08- | BREAST, LOBULAR | F | 71 | IV | N/A | N/A | ER(−)/ | Research | Normal | No | N/A |
| 090 | CARCINOMA | | | | | | PR(−)/ | | | Mutation | |
| | | | | | | | Her-2(−) | | | | |
| NA08- | BREAST, LOBULAR | F | 65 | I | N/A | N/A | ER(+)/ | Research | Mutation | PIK3CA | N/A |
| 174 | CARCINOMA | | | | | | PR(+)/ | | | H1047R | |
| | | | | | | | Her-2(−) | | | (3140A > G) | |
| NA08- | BREAST, LOBULAR | F | 68 | IV | N/A | N/A | ER(+)/ | Research | Mutation | PIK3CA | N/A |
| 184 | CARCINOMA | | | | | | PR(+)/ | | | E545K | |
| | | | | | | | Her-2(−) | | | (1633G > A) | |
| | | | | | | | | | | KRAS | |
| | | | | | | | | | | G12V | |
| | | | | | | | | | | (35G > T) | |
| NA09- | CERVIX, | F | 61 | IV | N/A | N/A | N/A | Research | Mutation | KRAS | N/A |
| 092 | ADENOCARCINOMA | | | | | | | | | G12D | |
| | | | | | | | | | | (35G > A) | |
| NA09- | CHRONIC | F | 27 | N/A | N/A | N/A | N/A | Research | Normal | No | N/A |
| 477 | MYELOPROLIFERATIVE | | | | | | | | | Mutation | |
| | DISORDER | | | | | | | | | | |
| NA09- | CHRONIC | F | 44 | N/A | N/A | N/A | N/A | Research | Mutation | JAK2 | N/A |
| 478 | MYELOPROLIFERATIVE | | | | | | | | | V617F | |
| | DISORDER | | | | | | | | | (1849G > T) | |
| NA09- | CHRONIC | F | 73 | N/A | N/A | N/A | N/A | Research | Mutation | JAK2 | N/A |
| 479 | MYELOPROLIFERATIVE | | | | | | | | | V617F | |
| | DISORDER | | | | | | | | | (1849G > T) | |
| NA09- | CHRONIC | M | 53 | N/A | N/A | N/A | N/A | Research | Mutation | JAK2 | N/A |
| 480 | MYELOPROLIFERATIVE | | | | | | | | | V617F | |
| | DISORDER | | | | | | | | | (1849G > T) | |
| NA09- | CHRONIC | F | 71 | N/A | N/A | N/A | N/A | Research | Normal | No | N/A |
| 481 | MYELOPROLIFERATIVE | | | | | | | | | Mutation | |
| | DISORDER | | | | | | | | | | |
| NA09- | CHRONIC | F | 81 | N/A | N/A | N/A | N/A | Research | Mutation | JAK2 | N/A |
| 482 | MYELOPROLIFERATIVE | | | | | | | | | V617F | |
| | DISORDER | | | | | | | | | (1849G > T) | |
| NA09- | CHRONIC | M | 62 | N/A | N/A | N/A | N/A | Research | Normal | No | N/A |
| 483 | MYELOPROLIFERATIVE | | | | | | | | | Mutation | |
| | DISORDER | | | | | | | | | | |
| NA09- | CHRONIC | M | 85 | N/A | N/A | N/A | N/A | Research | Normal | No | N/A |
| 484 | MYELOPROLIFERATIVE | | | | | | | | | Mutation | |
| | DISORDER | | | | | | | | | | |
| NA09- | CHRONIC | M | 45 | N/A | N/A | N/A | N/A | Research | Normal | No | N/A |
| 485 | MYELOPROLIFERATIVE | | | | | | | | | Mutation | |
| | DISORDER | | | | | | | | | | |
| NA09- | CHRONIC | M | 49 | N/A | N/A | N/A | N/A | Research | Normal | No | N/A |
| 486 | MYELOPROLIFERATIVE | | | | | | | | | Mutation | |
| | DISORDER | | | | | | | | | | |
| NA09- | COLORECTAL, | F | 56 | IV | N/A | N/A | N/A | Clincal | Normal | No | Negative |
| 222 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | COLORECTAL, | F | 61 | IV | N/A | N/A | N/A | Research | Normal | No | N/A |
| 058 | ADENOCARCINOMA | | | | | | | | | Mutation | |
| NA08- | COLORECTAL, | F | 60 | IV | N/A | N/A | N/A | Research | Normal | No | N/A |
| 062 | ADENOCARCINOMA | | | | | | | | | Mutation | |
| NA08- | COLORECTAL, | M | 89 | N/A | N/A | N/A | N/A | Research | Mutation | NRAS | N/A |
| 065 | ADENOCARCINOMA | | | | | | | | | Q61H | |
| | | | | | | | | | | (183A > T) | |
| | | | | | | | | | | TP53 | |
| | | | | | | | | | | R175H | |
| | | | | | | | | | | (524G > A) | |
| NA08- | COLORECTAL, | M | 63 | IV | N/A | N/A | N/A | Research | Mutation | KRAS | N/A |
| 064 | ADENOCARCINOMA | | | | | | | | | G12D | |
| | | | | | | | | | | (G35 > A) | |
| NA08- | COLORECTAL, | F | 63 | N/A | N/A | N/A | N/A | Research | Mutation | BRAF | N/A |
| 134 | ADENOCARCINOMA | | | | | | | | | V600E | |
| | | | | | | | | | | (1799T > A) | |
| NA08- | COLORECTAL, | M | 31 | IV | N/A | N/A | N/A | Research | Normal | No | N/A |
| 075 | ADENOCARCINOMA | | | | | | | | | Mutation | |
| NA08- | COLORECTAL, | F | 54 | N/A | N/A | N/A | N/A | Research | Mutation | PI3K | N/A |
| 071 | ADENOCARCINOMA | | | | | | | | | E545K | |
| | | | | | | | | | | (1633G > A) | |
| NA08- | COLORECTAL, | F | 56 | N/A | N/A | N/A | N/A | Research | Normal | No | N/A |
| 091 | ADENOCARCINOMA | | | | | | | | | Mutation | |
| NA09- | COLORECTAL, | F | 62 | N/A | N/A | N/A | N/A | Research | Normal | No | N/A |
| 094 | ADENOCARCINOMA | | | | | | | | | Mutation | |
| NA08- | COLORECTAL, | F | 52 | IV | N/A | N/A | N/A | Research | Mutation | APC | N/A |
| 106 | ADENOCARCINOMA | | | | | | | | | R1114* | |
| | | | | | | | | | | (3340C > T) | |
| NA08- | COLORECTAL, | M | 54 | N/A | N/A | N/A | N/A | Research | Normal | No | N/A |
| 092 | ADENOCARCINOMA | | | | | | | | | Mutation | |
| NA08- | COLORECTAL, | M | 51 | IV | N/A | N/A | N/A | Research | Mutation | KRAS | N/A |
| 072 | ADENOCARCINOMA | | | | | | | | | G13D | |
| | | | | | | | | | | (38G > A) | |
| | | | | | | | | | | TP53 | |
| | | | | | | | | | | R273H | |
| | | | | | | | | | | (818G > A) | |
| NA08- | COLORECTAL, | M | 67 | IV | N/A | N/A | N/A | Research | Mutation | KRAS | N/A |
| 076 | ADENOCARCINOMA | | | | | | | | | G12D | |
| | | | | | | | | | | (35G > A) | |
| NA08- | COLORECTAL, | M | 54 | N/A | N/A | N/A | N/A | Research | Mutation | KRAS | N/A |
| 104 | ADENOCARCINOMA | | | | | | | | | G12V | |
| | | | | | | | | | | (35G > T) | |
| | | | | | | | | | | PIK3CA | |
| | | | | | | | | | | E545K | |
| | | | | | | | | | | (1633G > A) | |
| NA08- | COLORECTAL, | F | 38 | IV | N/A | N/A | N/A | Research | Mutation | PIK3CA | N/A |
| 117 | ADENOCARCINOMA | | | | | | | | | R88Q | |
| | | | | | | | | | | (263G > A) | |
| | | | | | | | | | | KRAS | |
| | | | | | | | | | | G13D | |
| | | | | | | | | | | (38G > A) | |
| NA08- | COLORECTAL, | M | 65 | N/A | N/A | N/A | N/A | Research | Normal | No | N/A |
| 165 | ADENOCARCINOMA | | | | | | | | | Mutation | |
| NA08- | COLORECTAL, | M | 69 | IIIC | N/A | N/A | N/A | Research | Mutation | KRAS | N/A |
| 164 | ADENOCARCINOMA | | | | | | | | | G12V | |
| | | | | | | | | | | (35G > T) | |
| NA08- | COLORECTAL, | M | 64 | IIIC | N/A | N/A | N/A | Research | Normal | No | N/A |
| 162 | ADENOCARCINOMA | | | | | | | | | Mutation | |
| NA08- | COLORECTAL, | F | N/A | N/A | N/A | N/A | N/A | Research | Mutation | NRAS | N/A |
| 156 | ADENOCARCINOMA | | | | | | | | | G12D | |
| | | | | | | | | | | (35G > A) | |
| NA08- | COLORECTAL, | M | 72 | IV | N/A | N/A | N/A | Research | Normal | No | N/A |
| 167 | ADENOCARCINOMA | | | | | | | | | Mutation | |
| NA08- | COLORECTAL, | F | 53 | IV | N/A | N/A | N/A | Research | Mutation | KRAS | N/A |
| 198 | ADENOCARCINOMA | | | | | | | | | G12S | |
| | | | | | | | | | | (34G > A) | |
| NA08- | COLORECTAL, | M | 73 | IIIC | N/A | N/A | N/A | Research | Mutation | NRAS | N/A |
| 199 | ADENOCARCINOMA | | | | | | | | | G12D | |
| | | | | | | | | | | (35G > A) | |
| NA09- | COLORECTAL, | M | 67 | IV | N/A | N/A | N/A | Research | Normal | No | N/A |
| 006 | ADENOCARCINOMA | | | | | | | | | Mutation | |
| NA09- | COLORECTAL, | F | 56 | IV | N/A | N/A | N/A | Research | Mutation | KRAS | N/A |
| 101 | ADENOCARCINOMA | | | | | | | | | G13D (38G > | |
| | | | | | | | | | | A) | |
| NA09- | COLORECTAL, | | | | N/A | N/A | N/A | Research | Mutation | TP53 | N/A |
| 111 | ADENOCARCINOMA | | | | | | | | | R175H | |
| | | | | | | | | | | (524G > A) | |
| NA09- | COLORECTAL, | M | 36 | IV | N/A | N/A | N/A | Research | Mutation | KRAS | N/A |
| 262 | ADENOCARINOMA | | | | | | | | | G12C | |
| | | | | | | | | | | (34G > T) | |
| NA08- | COLORECTAL, | F | 55 | IV | N/A | N/A | N/A | Research | Normal | No | N/A |
| 105 | NEUROENDOCRINE | | | | | | | | | Mutation | |
| | CARCINOMA | | | | | | | | | | |
| NA08- | COLORECTAL, TUBULAR | F | 61 | IIB | N/A | N/A | N/A | Research | Normal | No | N/A |
| 073 | ADENOMA | | | | | | | | | Mutation | |
| NA08- | COLORECTAL, TUBULAR | M | 60 | IV | N/A | N/A | N/A | Research | Mutation | KRAS | N/A |
| 163 | ADENOMA | | | | | | | | | G12V | |
| | | | | | | | | | | (35G > T) | |
| NA09- | ESOPHAGUS, | M | 52 | IV | N/A | N/A | N/A | Clinical | Normal | No | Negative |
| 256 | SQUAMOUS CELL | | | | | | | | | Mutation | for |
| | CARCINOMA | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | GALL BLADDER, | F | 72 | IB | N/A | N/A | N/A | Research | Normal | No | N/A |
| 005 | ADENOCARCINOMA | | | | | | | | | Mutation | |
| NA08- | KIDNEY, RENAL CELL | M | 42 | IV | N/A | N/A | N/A | Research | Normal | No | N/A |
| 192 | CARCINOMA | | | | | | | | | Mutation | |
| NA08- | LIVER, | F | 58 | IV | N/A | N/A | N/A | Research | Normal | No | N/A |
| 061 | CHOLANGIOCARCINOMA | | | | | | | | | Mutation | |
| NA08- | LIVER, | M | 81 | IV | N/A | N/A | N/A | Research | Normal | No | N/A |
| 118 | CHOLANGIOCARCINOMA | | | | | | | | | Mutation | |
| NA08- | LIVER, | M | 74 | IIIB | N/A | N/A | N/A | Research | Normal | No | N/A |
| 160 | CHOLANGIOCARCINOMA | | | | | | | | | Mutation | |
| NA09- | LIVER, | F | 69 | IV | N/A | N/A | N/A | Research | Normal | No | N/A |
| 072 | CHOLANGIOCARCINOMA | | | | | | | | | Mutation | |
| NA09- | LIVER, | M | 44 | N/A | N/A | N/A | N/A | Research | Normal | No | N/A |
| 073 | CHOLANGIOCARCINOMA | | | | | | | | | Mutation | |
| NA09- | LIVER, | M | 39 | N/A | N/A | N/A | N/A | Research | Normal | No | N/A |
| 100 | CHOLANGIOCARCINOMA | | | | | | | | | Mutation | |
| NA09- | LUNG, | M | 43 | IV | F | 1 | N/A | Clinical | Mutation | EGFR | Positive |
| 129 | ADENOCARCINOMA | | | | | | | | | E746_A750 | for a 15 |
| | | | | | | | | | | del in frm | bp |
| | | | | | | | | | | 15 | deletion |
| | | | | | | | | | | (2236_50del) | in EGFR |
| | | | | | | | | | | | exon 19 |
| NA09- | LUNG, | M | 57 | IV | C | 34 | N/A | Clinical | Mutation | KRAS | Negative |
| 117 | ADENOCARCINOMA | | | | | | | | | G12D | for |
| | | | | | | | | | | (35G > A) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 71 | IIIA | N | 0 | N/A | Clinical | Normal | No | Negative |
| 120 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 77 | IV | C | 57 | N/A | Clinical | Mutation | KRAS | Negative |
| 128 | ADENOCARCINOMA | | | | | | | | | G12D | for |
| | | | | | | | | | | (35G > A) | insertions |
| | | | | | | | | | | TP53 | or |
| | | | | | | | | | | R248Q | deletions |
| | | | | | | | | | | (743G > A) | in EGFR |
| | | | | | | | | | | | exon 19 |
| NA09- | LUNG, | F | 73 | IB | F | 14 | N/A | Clinical | Mutation | KRAS | Negative |
| 127 | ADENOCARCINOMA | | | | | | | | | G12C | for |
| | | | | | | | | | | (34G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 58 | IB | F | 3 | N/A | Clinical | Normal | No | Negative |
| 125 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | N/A | N/A | F | 3 | N/A | Clinical | Normal | No | Negative |
| 126 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 72 | IV | F | 1 | N/A | Clinical | Normal | No | Negative |
| 132 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 75 | IV | F | 10 | N/A | Clinical | Normal | No | Negative |
| 131 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 48 | IV | N | 0 | N/A | Clinical | Normal | No | Negative |
| 139 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 53 | IV | F | 15 | N/A | Clinical | Mutation | KRAS | Negative |
| 135 | ADENOCARCINOMA | | | | | | | | | G12V | for |
| | | | | | | | | | | (35G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 49 | IV | N | 0 | N/A | Clinical | Normal | No | Negative |
| 138 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 56 | IA | N | 0 | N/A | Clinical | Normal | No | Negative |
| 149 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 56 | IB | F | 30 | N/A | Clinical | Mutation | KRAS | Negative |
| 150 | ADENOCARCINOMA | | | | | | | | | G12C | for |
| | | | | | | | | | | (34G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 38 | IV | C | 10 | N/A | Clinical | Normal | No | Negative |
| 151 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 69 | IV | N | 0 | N/A | Clinical | Mutation | EGFR | Positive |
| 155 | ADENOCARCINOMA | | | | | | | | | E746_A750 | for a |
| | | | | | | | | | | del in frm | 15 bp |
| | | | | | | | | | | 15 | deletion |
| | | | | | | | | | | (2236_50del) | in EGFR |
| | | | | | | | | | | | exon 19 |
| NA09- | LUNG, | M | 62 | IA | N | 0 | N/A | Clinical | Normal | No | Negative |
| 158 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 56 | IV | N | 0 | N/A | Clinical | Normal | No | Negative |
| 157 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 60 | IV | F | 25 | N/A | Clinical | Mutation | KRAS | N/A |
| 162 | ADENOCARCINOMA | | | | | | | | | G12C | |
| | | | | | | | | | | (34G > T) | |
| NA09- | LUNG, | M | 63 | IB | C | 45 | N/A | Clinical | Mutation | TP53 | Negative |
| 164 | ADENOCARCINOMA | | | | | | | | | R273L | for |
| | | | | | | | | | | (818G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 47 | IV | N | 0 | N/A | Clinical | Mutation | EGFR | Negative |
| 165 | ADENOCARCINOMA | | | | | | | | | L858R | for |
| | | | | | | | | | | (2573T > G) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 40 | IIIA | N | 0 | N/A | Clinical | Normal | No | Negative |
| 163 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 48 | IV | N | 0 | N/A | Clinical | Normal | No | Negative |
| 183 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 49 | IIIA | F | 20 | N/A | Clinical | Mutation | EGFR | Positive |
| 137 | ADENOCARCINOMA | | | | | | | | | E746_A750 | for a |
| | | | | | | | | | | del in frm | 15 bp |
| | | | | | | | | | | 15 | deletion |
| | | | | | | | | | | (2236_50del) | in EGFR |
| | | | | | | | | | | | exon 19 |
| NA09- | LUNG, | F | 54 | IV | N | 0 | N/A | Clinical | Mutation | EGFR | Positive |
| 184 | ADENOCARCINOMA | | | | | | | | | E746_A750 | for a |
| | | | | | | | | | | del in frm | 15 bp |
| | | | | | | | | | | 15 | deletion |
| | | | | | | | | | | (2235_49del) | in EGFR |
| | | | | | | | | | | | exon 19 |
| NA09- | LUNG, | F | 62 | IV | N | 0 | N/A | Clinical | Mutation | No | Positive |
| 190 | ADENOCARCINOMA | | | | | | | | | Mutation | for an |
| | | | | | | | | | | | 18 bp |
| | | | | | | | | | | | deletion |
| | | | | | | | | | | | in EGFR |
| | | | | | | | | | | | exon 19 |
| NA09- | LUNG, | F | 74 | IV | F | 10 | N/A | Clinical | Mutation | KRAS | Negative |
| 194 | ADENOCARCINOMA | | | | | | | | | G12C | for |
| | | | | | | | | | | (34G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 78 | IV | F | 40 | N/A | Clinical | Normal | No | Negative |
| 189 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 55 | IA | N | 0 | N/A | Clinical | Normal | No | Negative |
| 192 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 59 | IV | N | 0 | N/A | Clinical | Mutation | EGFR | Positive |
| 195 | ADENOCARCINOMA | | | | | | | | | E746_A750 | for a |
| | | | | | | | | | | del in frm | 15 bp |
| | | | | | | | | | | 15 | deletion |
| | | | | | | | | | | (2235_49del) | in EGFR |
| | | | | | | | | | | EGFR | exon 19 |
| | | | | | | | | | | T790M | |
| | | | | | | | | | | (2369C > T) | |
| | | | | | | | | | | TP53 | |
| | | | | | | | | | | R175H | |
| | | | | | | | | | | (524G > A) | |
| NA09- | LUNG, | F | 66 | IA | F | 30 | N/A | Clinical | Mutation | KRAS | Negative |
| 207 | ADENOCARCINOMA | | | | | | | | | 34G > T, | for |
| | | | | | | | | | | G12C | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 66 | IA | F | 30 | N/A | Clinical | Mutation | KRAS | Negative |
| 206 | ADENOCARCINOMA | | | | | | | | | 35G > C, | for |
| | | | | | | | | | | G12A | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 60 | IIIA | N | 0 | N/A | Clinical | Mutation | CTNNB1 | Positive |
| 261 | ADENOCARCINOMA | | | | | | | | | S37F | for a |
| | | | | | | | | | | (110C > T) | 15 bp |
| | | | | | | | | | | EGFR | deletion |
| | | | | | | | | | | E746_A750 | in EGFR |
| | | | | | | | | | | del in frm | exon 19 |
| | | | | | | | | | | 15 | |
| | | | | | | | | | | (2235_49del) | |
| NA09- | LUNG, | F | 57 | IV | N | 0 | N/A | Clinical | Normal | No | Negative |
| 219 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 73 | IIIA | F | 37 | N/A | Clinical | Mutation | KRAS | Negative |
| 220 | ADENOCARCINOMA | | | | | | | | | 35G > T, | for |
| | | | | | | | | | | G12V | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 76 | IIIB | F | 10 | N/A | Clinical | Normal | No | Negative |
| 258 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 68 | IV | N | 0 | N/A | Clinical | Mutation | EGFR | Negative |
| 240 | ADENOCARCINOMA | | | | | | | | | L858R | for |
| | | | | | | | | | | (2575T > G) | insertions |
| | | | | | | | | | | EGFR | or |
| | | | | | | | | | | T790M | deletions |
| | | | | | | | | | | (2369C > T) | in EGFR |
| | | | | | | | | | | | exon 19 |
| NA09- | LUNG, | F | 62 | IV | C | 100 | N/A | Clinical | Mutation | KRAS | Negative |
| 253 | ADENOCARCINOMA | | | | | | | | | G12C (34G > | for |
| | | | | | | | | | | T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 74 | IIB | N | 0 | N/A | Clinical | Normal | No | Negative |
| 235 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 49 | IV | N | 0 | N/A | Clinical | Mutation | EGFR | Positive |
| 237 | ADENOCARCINOMA | | | | | | | | | E746_A750 | for a |
| | | | | | | | | | | del in frm | 15 bp |
| | | | | | | | | | | 15 | deletion |
| | | | | | | | | | | (2235_49del) | in EGFR |
| | | | | | | | | | | | exon 19 |
| NA09- | LUNG, | F | 54 | IV | F | 15 | N/A | Clinical | Mutation | KRAS | Negative |
| 234 | ADENOCARCINOMA | | | | | | | | | G12C | for |
| | | | | | | | | | | (34G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 86 | IV | N | 0 | N/A | Clinical | Normal | No | Negative |
| 241 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | F | 76 | IV | N | 0 | N/A | Clinical | Mutation | EGFR | Positive |
| 238 | ADENOCARCINOMA | | | | | | | | | E746_A750 | for a |
| | | | | | | | | | | del in frm | 15 bp |
| | | | | | | | | | | 15 | deletion |
| | | | | | | | | | | (2235_49del) | in EGFR |
| | | | | | | | | | | | exon 19 |
| NA09- | LUNG, | F | N/A | N/A | N | 0 | N/A | Clinical | Normal | No | Negative |
| 290 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, | M | 72 | IA | F | 45 | N/A | Clinical | Mutation | KRAS | Negative |
| 291 | ADENOCARCINOMA | | | | | | | | | G13D | for |
| | | | | | | | | | | (38G > A) | insertions |
| | | | | | | | | | | | or |
| | | | | | | | | | | | deletions |
| | | | | | | | | | | | in EGFR |
| | | | | | | | | | | | exon 19 |
| NA08- | LUNG, | M | 68 | IV | F | 20 | N/A | Research | Mutation | EGFR | N/A |
| 056 | ADENOCARCINOMA | | | | | | | | | L858R | |
| | | | | | | | | | | (2573T > | |
| | | | | | | | | | | G) | |
| NA08- | LUNG, | F | 45 | IV | F | 20 | N/A | Research | Normal | No | N/A |
| 112 | ADENOCARCINOMA | | | | | | | | | Mutation | |
| NA08- | LUNG, | F | 49 | IV | N | 0 | N/A | Research | Normal | No | N/A |
| 172 | ADENOCARCINOMA | | | | | | | | | Mutation | |
| NA08- | LUNG, | F | 54 | IIIA | F | 2 | N/A | Research | Normal | No | N/A |
| 191 | ADENOCARCINOMA | | | | | | | | | Mutation | |
| NA08- | LUNG, | M | 44 | IIIA | F | 2 | N/A | Research | Normal | No | N/A |
| 237 | ADENOCARCINOMA | | | | | | | | | Mutation | |
| NA08- | LUNG, | F | 74 | IB | N | 0 | N/A | Research | Mutation | EGFR | N/A |
| 220 | ADENOCARCINOMA | | | | | | | | | L858R | |
| | | | | | | | | | | (2573T > | |
| | | | | | | | | | | G) | |
| NA08- | LUNG, | M | 58 | IV | N | 0 | N/A | Research | Normal | No | N/A |
| 238 | ADENOCARCINOMA | | | | | | | | | Mutation | |
| NA09- | LUNG, | M | 22 | IV | N | 0 | N/A | Research | Normal | No | N/A |
| 025 | ADENOCARCINOMA | | | | | | | | | Mutation | |
| NA09- | LUNG, | F | 48 | IIIA | F | 10 | N/A | Research | Normal | No | N/A |
| 026 | ADENOCARCINOMA | | | | | | | | | Mutation | |
| NA09- | LUNG, | F | N/A | N/A | F | N/A | N/A | Research | Normal | No | N/A |
| 236 | ADENOCARCINOMA | | | | | | | | | Mutation | |
| NA09- | LUNG, | F | 74 | IB | N | 0 | N/A | Research | Normal | No | N/A |
| 292 | ADENOCARCINOMA | | | | | | | | | Mutation | |
| NA09- | LUNG, | F | 59 | IA | C | 60 | N/A | Research | Mutation | NRAS | N/A |
| 302 | ADENOCARCINOMA | | | | | | | | | Q61L | |
| | | | | | | | | | | (182A > T) | |
| | | | | | | | | | | TP53 | |
| | | | | | | | | | | R248P | |
| | | | | | | | | | | (743G > C) | |
| NA09- | LUNG, | F | 63 | IIIA | N | 0 | N/A | Research | Normal | No | N/A |
| 303 | ADENOCARCINOMA | | | | | | | | | Mutation | |
| NA09- | LUNG, | F | 44 | IIIA | C | 30 | N/A | Research | Mutation | KRAS | N/A |
| 304 | ADENOCARCINOMA | | | | | | | | | G12V | |
| | | | | | | | | | | (35G > T) | |
| NA09- | LUNG, | M | 64 | IB | C | 50 | N/A | Research | Normal | No | N/A |
| 306 | ADENOCARCINOMA | | | | | | | | | Mutation | |
| NA09- | LUNG, | F | 66 | IA | F | 8 | N/A | Research | Mutation | KRAS | N/A |
| 307 | ADENOCARCINOMA | | | | | | | | | G12C | |
| | | | | | | | | | | (34G > T) | |
| NA09- | LUNG, | F | 60 | IB | C | 43 | N/A | Research | Normal | No | N/A |
| 293 | ADENOCARCINOMA | | | | | | | | | Mutation | |
| NA09- | LUNG, | F | 60 | IB | F | 50 | N/A | Research | Normal | No | N/A |
| 294 | ADENOCARCINOMA | | | | | | | | | Mutation | |
| NA09- | LUNG, | F | 62 | IB | F | N/A | N/A | Research | Normal | No | N/A |
| 295 | ADENOCARCINOMA | | | | | | | | | Mutation | |
| NA09- | LUNG, | F | 75 | IB | N | 0 | N/A | Research | Mutation | TP53 | N/A |
| 296 | ADENOCARCINOMA | | | | | | | | | R248Q | |
| | | | | | | | | | | (743G > A) | |
| NA08- | LUNG, | F | 20 | N/A | N | 0 | N/A | Research | Normal | No | N/A |
| 051 | NEUROENDOCRINE | | | | | | | | | Mutation | |
| | CARCINOMA | | | | | | | | | | |
| NA08- | LUNG, | M | 69 | N/A | N | 0 | N/A | Research | Normal | No | N/A |
| 052 | NEUROENDOCRINE | | | | | | | | | Mutation | |
| | CARCINOMA | | | | | | | | | | |
| NA09- | LUNG, NON-SMALL CELL | F | 55 | IV | N | 0 | N/A | Clinical | Normal | No | Negative |
| 156 | LUNG CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | M | 56 | IV | N | 0 | N/A | Clinical | Normal | No | Negative |
| 166 | LUNG CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | M | 76 | IIIB | F | 40 | N/A | Clinical | Normal | No | Negative |
| 186 | LUNG CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | F | 81 | IV | N | 0 | N/A | Clinical | Normal | No | Negative |
| 191 | LUNG CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | F | 66 | N/A | F | N/A | N/A | Clinical | Mutation | EGFR | Negative |
| 187 | LUNG CARCINOMA | | | | | | | | | L858R | for |
| | | | | | | | | | | (2573T > G) | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | F | 55 | IV | C | 30 | N/A | Clinical | Mutation | KRAS | Negative |
| 188 | LUNG CARCINOMA | | | | | | | | | G12C | for |
| | | | | | | | | | | (34G > T) | insertions |
| | | | | | | | | | | | or |
| | | | | | | | | | | | deletions |
| | | | | | | | | | | | in EGFR |
| | | | | | | | | | | | exon 19 |
| NA09- | LUNG, NON-SMALL CELL | M | 77 | IV | N | 0 | N/A | Clinical | Normal | No | Negative |
| 338 | LUNG CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | LUNG, NON-SMALL CELL | F | 76 | IV | N | 0 | N/A | Research | Normal | No | N/A |
| 196 | LUNG CARCINOMA | | | | | | | | | Mutation | |
| NA09- | LUNG, NON-SMALL CELL | F | 65 | IV | F | 5 | N/A | Research | Normal | No | N/A |
| 061 | LUNG CARCINOMA | | | | | | | | | Mutation | |
| NA09- | LUNG, SQUAMOUS CELL | F | 83 | IV | F | 100 | N/A | Research | Mutation | KRAS | N/A |
| 023 | CARCINOMA | | | | | | | | | G12C | |
| | | | | | | | | | | (34G > T) | |
| NA09- | LUNG, SQUAMOUS CELL | M | 86 | IIB | C | 65 | N/A | Research | Normal | No | N/A |
| 301 | CARCINOMA | | | | | | | | | Mutation | |
| NA09- | LUNG, SQUAMOUS CELL | M | 76 | IB | C | 120 | N/A | Research | Normal | No | N/A |
| 305 | CARCINOMA | | | | | | | | | Mutation | |
| NA09- | LUNG, SQUAMOUS CELL | F | 75 | IB | F | 80 | N/A | Research | Normal | No | N/A |
| 308 | CARCINOMA | | | | | | | | | Mutation | |
| NA09- | LUNG, SQUAMOUS CELL | F | 79 | IIB | F | 120 | N/A | Research | Normal | No | N/A |
| 309 | CARCINOMA | | | | | | | | | Mutation | |
| NA09- | LUNG, SQUAMOUS CELL | M | 62 | IIIA | F | 40 | N/A | Research | Mutation | KRAS | N/A |
| 310 | CARCINOMA | | | | | | | | | G12A | |
| | | | | | | | | | | (35G > C) | |
| NA09- | LUNG, SQUAMOUS CELL | M | 51 | IIA | C | 33 | N/A | Research | Normal | No | N/A |
| 311 | CARCINOMA | | | | | | | | | Mutation | |
| NA09- | LUNG, SQUAMOUS CELL | M | 73 | IB | F | 50 | N/A | Research | Normal | No | N/A |
| 297 | CARCINOMA | | | | | | | | | Mutation | |
| NA09- | LUNG, SQUAMOUS CELL | M | 79 | IB | C | 65 | N/A | Research | Normal | No | N/A |
| 298 | CARCINOMA | | | | | | | | | Mutation | |
| NA09- | LUNG, SQUAMOUS CELL | M | 62 | IA | C | 30 | N/A | Research | Normal | No | N/A |
| 299 | CARCINOMA | | | | | | | | | Mutation | |
| NA09- | LUNG, SQUAMOUS CELL | F | 75 | IB | C | 55 | N/A | Research | Mutation | PIK3CA | N/A |
| 300 | CARCINOMA | | | | | | | | | E542K | |
| | | | | | | | | | | (1624G > A) | |
| NA09- | MEDIASTINUM, LARGE | F | 35 | N/A | N/A | N/A | N/A | Clinical | Normal | No | Negative |
| 336 | CELL | | | | | | | | | Mutation | for |
| | NEUROENDOCRINE | | | | | | | | | | insertions |
| | CARCINOMA | | | | | | | | | | or |
| | | | | | | | | | | | deletions |
| | | | | | | | | | | | in EGFR |
| | | | | | | | | | | | exon 19 |
| NA09- | MELANOMA | F | 41 | N/A | N/A | N/A | N/A | Research | Mutation | NRAS | N/A |
| 037 | | | | | | | | | | Q61R | |
| | | | | | | | | | | (182A > G) | |
| NA09- | MELANOMA | M | 52 | IIB | N/A | N/A | N/A | Research | Mutation | BRAF | N/A |
| 045 | | | | | | | | | | V600M | |
| | | | | | | | | | | (1798G > A) | |
| NA09- | MELANOMA | F | 52 | IV | N/A | N/A | N/A | Research | Mutation | BRAF | N/A |
| 041 | | | | | | | | | | V600E | |
| | | | | | | | | | | (1799T > A) | |
| NA09- | MELANOMA | M | 83 | IV | N/A | N/A | N/A | Research | Mutation | NRAS | N/A |
| 047 | | | | | | | | | | Q61L | |
| | | | | | | | | | | (182A > T) | |
| NA09- | MELANOMA | M | 58 | IV | N/A | N/A | N/A | Research | Mutation | BRAF | N/A |
| 046 | | | | | | | | | | V600E | |
| | | | | | | | | | | (1799T > A) | |
| NA09- | MELANOMA | M | 67 | IIIC | N/A | N/A | N/A | Research | Mutation | BRAF | N/A |
| 050 | | | | | | | | | | V600E | |
| | | | | | | | | | | (1799T > A) | |
| NA09- | MELANOMA | F | 60 | IV | N/A | N/A | N/A | Research | Normal | No | N/A |
| 038 | | | | | | | | | | Mutation | |
| NA09- | MELANOMA | M | 57 | I-III | N/A | N/A | N/A | Research | Mutation | BRAF | N/A |
| 039 | | | | | | | | | | V600E | |
| | | | | | | | | | | (1799T > A) | |
| NA09- | MELANOMA | M | 73 | III | N/A | N/A | N/A | Research | Normal | No | N/A |
| 043 | | | | | | | | | | Mutation | |
| NA09- | MELANOMA | F | 68 | II | N/A | N/A | N/A | Research | Normal | No | N/A |
| 040 | | | | | | | | | | Mutation | |
| NA08- | MELANOMA, OCULAR | M | 33 | N/A | N/A | N/A | N/A | Research | Normal | No | N/A |
| 155 | | | | | | | | | | Mutation | |
| NA09- | OVARY, | F | 75 | N/A | N/A | N/A | N/A | Clinical | Normal | No | Negative |
| 121 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA09- | OVARY, | M | 72 | IIA | N/A | N/A | N/A | Research | Normal | No | N/A |
| 218 | ADENOCARCINOMA | | | | | | | | | Mutation | |
| NA08- | OVARY, SMALL CELL | F | 21 | IC | N/A | N/A | N/A | Research | Normal | No | N/A |
| 055 | CARCINOMA, | | | | | | | | | Mutation | |
| | HYPERCALCEMIC TYPE | | | | | | | | | | |
| NA08- | PANCREAS, ACINAL | F | 71 | N/A | C | 183 | N/A | Research | Normal | No | N/A |
| 107 | CELL CARCINOMA | | | | | | | | | Mutation | |
| NA09- | PANCREAS, DUCTAL | F | 62 | N/A | N | 0 | N/A | Clinical | Mutation | KRAS | Negative |
| 193 | ADENOCARCINOMA | | | | | | | | | G12V | for |
| | | | | | | | | | | (35G > T) | insertions |
| | | | | | | | | | | | or |
| | | | | | | | | | | | deletions |
| | | | | | | | | | | | in EGFR |
| | | | | | | | | | | | exon 19 |
| NA09- | PANCREAS, DUCTAL | M | 78 | N/A | F | N/A | N/A | Clinical | Mutation | KRAS | Negative |
| 232 | ADENOCARCINOMA | | | | | | | | | G12V | for |
| | | | | | | | | | | (35G > T) | insertions |
| | | | | | | | | | | | or |
| | | | | | | | | | | | deletions |
| | | | | | | | | | | | in EGFR |
| | | | | | | | | | | | exon 19 |
| NA08- | PANCREAS, DUCTAL | F | 48 | IV | F | 20 | N/A | Research | Normal | No | N/A |
| 060 | ADENOCARCINOMA | | | | | | | | | Mutation | |
| NA08- | PANCREAS, DUCTAL | M | 49 | IV | C | 156 | N/A | Research | Mutation | KRAS | N/A |
| 074 | ADENOCARCINOMA | | | | | | | | | G12R | |
| | | | | | | | | | | (34G > C) | |
| NA08- | PANCREAS, DUCTAL | F | 77 | IV | N | 0 | N/A | Research | Normal | No | N/A |
| 099 | ADENOCARCINOMA | | | | | | | | | Mutation | |
| NA08- | PANCREAS, DUCTAL | F | 77 | IIA | N | 0 | N/A | Research | Normal | No | N/A |
| 098 | ADENOCARCINOMA | | | | | | | | | Mutation | |
| NA08- | PANCREAS, DUCTAL | F | 68 | IB | N | 0 | N/A | Research | Normal | No | N/A |
| 096 | ADENOCARCINOMA | | | | | | | | | Mutation | |
| NA08- | PANCREAS, DUCTAL | F | 57 | IV | F | 548 | N/A | Research | Mutation | KRAS | N/A |
| 069 | ADENOCARCINOMA | | | | | | | | | G12D | |
| | | | | | | | | | | (35G > A) | |
| NA08- | PANCREAS, DUCTAL | F | 64 | IV | F | 30 | N/A | Research | Mutation | KRAS | N/A |
| 100 | ADENOCARCINOMA | | | | | | | | | G12V | |
| | | | | | | | | | | (35G > T) | |
| | | | | | | | | | | TP53 | |
| | | | | | | | | | | R248Q | |
| | | | | | | | | | | (743G > A) | |
| NA08- | PANCREAS, DUCTAL | M | 64 | IIB | C | 913 | N/A | Research | Mutation | KRAS | N/A |
| 097 | ADENOCARCINOMA | | | | | | | | | G12V | |
| | | | | | | | | | | (35G > T) | |
| NA08- | PANCREAS, DUCTAL | M | 55 | N/A | F | 365 | N/A | Research | Mutation | KRAS | N/A |
| 093 | ADENOCARCINOMA | | | | | | | | | G12R | |
| | | | | | | | | | | (34G > C) | |
| NA08- | PANCREAS, DUCTAL | M | 68 | IV | N | 0 | N/A | Research | Normal | No | N/A |
| 108 | ADENOCARCINOMA | | | | | | | | | Mutation | |
| NA08- | PANCREAS, DUCTAL | M | 53 | N/A | C | 365 | N/A | Research | Normal | No | N/A |
| 158 | ADENOCARCINOMA | | | | | | | | | Mutation | |
| NA08- | PANCREAS, DUCTAL | F | 47 | N/A | C | 183 | N/A | Research | Mutation | KRAS | N/A |
| 193 | ADENOCARCINOMA | | | | | | | | | G12D | |
| | | | | | | | | | | (35G > A) | |
| NA08- | PANCREAS, DUCTAL | M | 57 | IIB | F | 40 | N/A | Research | Mutation | KRAS | N/A |
| 170 | ADENOCARCINOMA | | | | | | | | | G12D | |
| | | | | | | | | | | (35G > A) | |
| | | | | | | | | | | TP53 | |
| | | | | | | | | | | R175H | |
| | | | | | | | | | | (524G > A) | |
| NA08- | PANCREAS, DUCTAL | M | 84 | IB | F | 548 | N/A | Research | Normal | No | N/A |
| 166 | ADENOCARCINOMA | | | | | | | | | Mutation | |
| NA08- | PANCREAS, DUCTAL | M | 82 | N/A | F | 52 | N/A | Research | Mutation | KRAS | N/A |
| 169 | ADENOCARCINOMA | | | | | | | | | G12V | |
| | | | | | | | | | | (35G > T) | |
| NA08- | PANCREAS, DUCTAL | F | 47 | N/A | N | 0 | N/A | Research | Normal | No | N/A |
| 177 | ADENOCARCINOMA | | | | | | | | | Mutation | |
| NA08- | PANCREAS, DUCTAL | M | 56 | IV | F | 30 | N/A | Research | Mutation | KRAS | N/A |
| 212 | ADENOCARCINOMA | | | | | | | | | G12V | |
| | | | | | | | | | | (35G > T) | |
| NA08- | PANCREAS, | M | 71 | IV | N | 0 | N/A | Research | Normal | No | N/A |
| 063 | NEUROENDOCRINE | | | | | | | | | Mutation | |
| | CARCINOMA | | | | | | | | | | |
| NA08- | PANCREAS, | M | 60 | IV | F | N/A | N/A | Research | Normal | No | N/A |
| 068 | NEUROENDOCRINE | | | | | | | | | Mutation | |
| | CARCINOMA | | | | | | | | | | |
| NA09- | PANCREAS, | M | 31 | N/A | N | 0 | N/A | Clinical | Normal | No | Negative |
| 225 | PANCREATOBLASTOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | PANCREATOBILIARY | F | 48 | IV | N/A | N/A | N/A | Research | Normal | No | N/A |
| 161 | ADENOCARCINOMA | | | | | | | | | Mutation | |
| NA09- | PITUITARY, CARCINOMA | N/A | N/A | N/A | N/A | N/A | N/A | Research | Normal | No | N/A |
| 118 | | | | | | | | | | Mutation | |
| NA09- | PROSTATE, | M | 60 | N/A | N/A | N/A | N/A | Research | Normal | No | N/A |
| 268 | ADENOCARCINOMA | | | | | | | | | Mutation | |
| NA09- | PROSTATE, | M | 49 | N/A | N/A | N/A | N/A | Research | Normal | No | N/A |
| 277 | ADENOCARCINOMA | | | | | | | | | Mutation | |
| NA09- | PROSTATE, | M | 59 | N/A | N/A | N/A | N/A | Research | Normal | No | N/A |
| 278 | ADENOCARCINOMA | | | | | | | | | Mutation | |
| NA09- | PROSTATE, | M | 80 | N/A | N/A | N/A | N/A | Research | Mutation | KRAS | N/A |
| 279 | ADENOCARCINOMA | | | | | | | | | G13R | |
| | | | | | | | | | | (37G > C) | |
| NA09- | PROSTATE, | M | 55 | N/A | N/A | N/A | N/A | Research | Normal | No | N/A |
| 280 | ADENOCARCINOMA | | | | | | | | | Mutation | |
| NA09- | PROSTATE, | M | 90 | N/A | N/A | N/A | N/A | Research | Normal | No | N/A |
| 281 | ADENOCARCINOMA | | | | | | | | | Mutation | |
| NA09- | PROSTATE, | M | 57 | N/A | N/A | N/A | N/A | Research | Normal | No | N/A |
| 282 | ADENOCARCINOMA | | | | | | | | | Mutation | |
| NA09- | PROSTATE, | M | 56 | N/A | N/A | N/A | N/A | Research | Normal | No | N/A |
| 283 | ADENOCARCINOMA | | | | | | | | | Mutation | |
| NA09- | PROSTATE, | M | 58 | N/A | N/A | N/A | N/A | Research | Normal | No | N/A |
| 284 | ADENOCARCINOMA | | | | | | | | | Mutation | |
| NA09- | PROSTATE, | M | 65 | N/A | N/A | N/A | N/A | Research | Normal | No | N/A |
| 285 | ADENOCARCINOMA | | | | | | | | | Mutation | |
| NA09- | PROSTATE, | M | 51 | N/A | N/A | N/A | N/A | Research | Normal | No | N/A |
| 286 | ADENOCARCINOMA | | | | | | | | | Mutation | |
| NA09- | PROSTATE, | M | 60 | N/A | N/A | N/A | N/A | Research | Normal | No | N/A |
| 269 | ADENOCARCINOMA | | | | | | | | | Mutation | |
| NA09- | PROSTATE, | M | 63 | N/A | N/A | N/A | N/A | Research | Mutation | CTNNB1 | N/A |
| 287 | ADENOCARCINOMA | | | | | | | | | S33C | |
| | | | | | | | | | | (98C > G) | |
| NA09- | PROSTATE, | M | 48 | N/A | N/A | N/A | N/A | Research | Normal | No | N/A |
| 270 | ADENOCARCINOMA | | | | | | | | | Mutation | |
| NA09- | PROSTATE, | M | 65 | N/A | N/A | N/A | N/A | Research | Mutation | CTNNB1 | N/A |
| 271 | ADENOCARCINOMA | | | | | | | | | S37Y | |
| | | | | | | | | | | (110C > A) | |
| | | | | | | | | | | PIK3CA | |
| | | | | | | | | | | E542K | |
| | | | | | | | | | | (1624G > A) | |
| NA09- | PROSTATE, | M | 58 | N/A | N/A | N/A | N/A | Research | Normal | No | N/A |
| 272 | ADENOCARCINOMA | | | | | | | | | Mutation | |
| NA09- | PROSTATE, | M | 60 | N/A | N/A | N/A | N/A | Research | Normal | No | N/A |
| 273 | ADENOCARCINOMA | | | | | | | | | Mutation | |
| NA09- | PROSTATE, | M | 69 | N/A | N/A | N/A | N/A | Research | Normal | No | N/A |
| 274 | ADENOCARCINOMA | | | | | | | | | Mutation | |
| NA09- | PROSTATE, | M | 58 | N/A | N/A | N/A | N/A | Research | Normal | No | N/A |
| 275 | ADENOCARCINOMA | | | | | | | | | Mutation | |
| NA09- | PROSTATE, | M | 85 | N/A | N/A | N/A | N/A | Research | Normal | No | N/A |
| 276 | ADENOCARCINOMA | | | | | | | | | Mutation | |
| NA09- | SALIVARY GLAND, | F | 61 | IVC | N/A | N/A | N/A | Clinical | Normal | No | Negative |
| 181 | ADENOID CYSTIC | | | | | | | | | Mutation | for |
| | CARCINOMA | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | SALIVARY GLAND, | M | 52 | N/A | N/A | N/A | N/A | Research | Normal | No | N/A |
| 110 | ADENOID CYSTIC | | | | | | | | | Mutation | |
| | CARCINOMA | | | | | | | | | | |
| NA09- | SALIVARY GLAND, | M | 71 | N/A | N/A | N/A | N/A | Research | Normal | No | N/A |
| 239 | ADENOID CYSTIC | | | | | | | | | Mutation | |
| | CARCINOMA | | | | | | | | | | |
| NA08- | SINOPHARYNX, | M | 65 | N/A | N/A | N/A | N/A | Research | Normal | No | N/A |
| 059 | SINONASAL | | | | | | | | | Mutation | |
| | UNDIFFERENTIATED | | | | | | | | | | |
| | CARCINOMA | | | | | | | | | | |
| NA08- | SMALL INTESTINE, | F | 51 | IV | N/A | N/A | N/A | Research | Normal | No | N/A |
| 118 | ADENOCARCINOMA | | | | | | | | | Mutation | |
| NA08- | SMALL INTESTINE, | M | 59 | N/A | N/A | N/A | N/A | Research | Normal | No | N/A |
| 109 | ADENOCARCINOMA | | | | | | | | | Mutation | |
| NA08- | SOFT TISSUE, | F | 54 | N/A | N/A | N/A | N/A | Research | Normal | No | N/A |
| 053 | LEIOMYOSARCOMA | | | | | | | | | Mutation | |
| NA09- | SOFT TISSUE, | F | 76 | N/A | N/A | N/A | N/A | Research | Normal | No | N/A |
| 007 | MYXOFIBROSARCOMA | | | | | | | | | Mutation | |
| NA09- | STOMACH, | F | 72 | IV | N/A | N/A | N/A | Clinical | Normal | No | Negative |
| 221 | ADENOCARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | STOMACH, | F | 50 | N/A | N/A | N/A | N/A | Research | Normal | No | N/A |
| 095 | ADENOCARCINOMA | | | | | | | | | Mutation | |
| NA08- | STOMACH, | F | 72 | N/A | N/A | N/A | N/A | Research | Normal | No | N/A |
| 070 | ADENOCARCINOMA | | | | | | | | | Mutation | |
| NA08- | STOMACH, | M | 77 | IV | N/A | N/A | N/A | Research | Normal | No | N/A |
| 234 | ADENOCARCINOMA | | | | | | | | | Mutation | |
| NA09- | STOMACH, | M | N/A | N/A | N/A | N/A | N/A | Research | Normal | No | N/A |
| 152 | NEUROBLASTOMA | | | | | | | | | Mutation | |
| NA09- | THYMUS, CARCINOMA | F | 66 | N/A | N/A | N/A | N/A | Research | Normal | No | N/A |
| 110 | | | | | | | | | | Mutation | |
| NA09- | THYROID, HURTHLE | F | | N/A | N/A | N/A | N/A | Research | Mutation | TP53 | N/A |
| 024 | CELL CARCINOMA | | | | | | | | | R306* | |
| | | | | | | | | | | (916C > T) | |
| NA09- | THYROID, PAPILLARY | F | 52 | N/A | N/A | N/A | N/A | Clinical | Normal | No | Negative |
| 148 | CARCINOMA | | | | | | | | | Mutation | for |
| | | | | | | | | | | | insertions |
| | | | | | | | | | | | or |
| | | | | | | | | | | | deletions |
| | | | | | | | | | | | in EGFR |
| | | | | | | | | | | | exon 19 |
| NA08- | THYROID, PAPILLARY | F | 12 | N/A | N/A | N/A | N/A | Research | Normal | No | N/A |
| 180 | CARCINOMA | | | | | | | | | Mutation | |

TABLE 5
Mutation distribution across tumor types

| | Cancer Genes | | | | | | | | | | | | |
|---|---|---|---|---|---|---|---|---|---|---|---|---|---|
| Tumor type | APC | BRAF | CTNNB1 | EGFR | FLT3 | JAK2 | KIT | KRAS | NOTCH1 | NRAS | PIK3CA | PTEN | TP53 |
| Breast | 0% | 0% | 0% | 0% | 0% | 0% | 0% | 3% | 0% | 0% | 12% | 0% | 6% |
| | 4% | 3% | 2% | <1% | 0% | 0% | 0% | 5% | 2% | 1% | 25% | 5% | 55% |
| CMD | 0% | 0% | 0% | 0% | 0% | 40% | 0% | 0% | 0% | 0% | 0% | 0% | 0% |
| | N/A | N/A | N/A | N/A | N/A | 52% | 10% | N/A | N/A | N/A | N/A | N/A | N/A |
| Colorectal | 3% | 3% | 0% | 0% | 0% | 0% | 0% | 33% | 0% | 10% | 10% | 0% | 10% |
| | 39% | 11% | 5% | <1% | 0% | 0% | 1% | 32% | 2% | 3% | 14% | 13% | 42% |
| Lung | 0% | 0% | 1% | 17% | 0% | 0% | 0% | 21% | 0% | 1% | 1% | 0% | 0% |
| | 1% | 2% | 3% | 26% | <1% | 0% | 0% | 17% | 1% | 1% | 3% | 9% | 64% |
| Melanoma | 0% | 45% | 0% | 0% | 0% | 0% | 0% | 0% | 0% | 18% | 0% | 0% | 0% |
| | 4% | 42% | 6% | 1% | 0% | 0% | 9% | 2% | 0% | 20% | 3% | 18% | 27% |
| Pancreatic | 0% | 0% | 0% | 0% | 0% | 0% | 0% | 48% | 0% | 0% | 0% | 0% | 9% |
| | 13% | 3% | 23% | <1% | 0% | 0% | 0% | 67% | 0% | 2% | 6% | 1% | 68% |
| Prostate | 0% | 0% | 10% | 0% | 0% | 0% | 0% | 5% | 0% | 0% | 5% | 0% | 0% |
| | 7% | 6% | 7% | 6% | 0% | 0% | 0% | 8% | 0% | 2% | 2% | 13% | 80% |

% values: top (our data); bottom (previous reports)

TABLE 6
Assessment of Sample Heterogeneity in Primary Tumors

| | ESTIMATED % | % MUTANT = | | RESPONSE |
|---|---|---|---|---|
| SAMPLE_ID | TUMOR CELLS | MUT * 100/(WT + MUT) | MUTATION(S) | TO EGFR TKIs |
| NA09-261 | N/A | 30% (CTNNB1 S37F) | CTNNB1 S37F (110C > T) | UNKNOWN |
| | | 22% (EGFR E746_A750) | EGFR E746_A750 del in frm 15 (2235_49del) | |
| NA09-137 | *10-20% | 7% | EGFR E746_A750 del in frm 15 (2235_49del) | UNKNOWN |
| NA09-184 | 60% | 17% | EGFR E746_A750 del in frm 15 (2235_49del) | YES |
| NA09-237 | N/A | 59% | EGFR E746_A750 del in frm 15 (2235_49del) | YES |
| NA09-238 | *60% | 74% | EGFR E746_A750 del in frm 15 (2235_49del) | UNKNOWN |
| NA09-129 | 60% | 14% | EGFR E746_A750 del in frm 15 (2236_50del) | YES |
| NA09-155 | 40% | 5% | EGFR E746_A750 del in frm 15 (2236_50del) | UNKNOWN |
| NA09-165 | 10-20% | 12% | EGFR L858R (2573T > G) | YES |
| NA09-187 | N/A | 13% | EGFR L858R (2573T > G) | UNKNOWN |
| NA09-206 | 40% | 6% | KRAS G12A (35G > C) | NOT APPLICABLE |
| NA09-127 | 10-20% | 41% | KRAS G12C (34G > T) | NOT APPLICABLE |
| NA09-150 | N/A | 35% | KRAS G12C (34G > T) | NOT APPLICABLE |
| NA09-162 | N/A | 57% | KRAS G12C (34G > T) | NOT APPLICABLE |
| NA09-188 | 30% | 26% | KRAS G12C (34G > T) | NOT APPLICABLE |
| NA09-194 | 25-30% | 33% | KRAS G12C (34G > T) | NOT APPLICABLE |
| NA09-207 | 60% | 49% | KRAS G12C (34G > T) | NOT APPLICABLE |
| NA09-234 | *10-20% | 19% | KRAS G12C (34G > T) | NOT APPLICABLE |
| NA09-253 | 70-80% | 66% | KRAS G12C (34G > T) | NOT APPLICABLE |
| NA09-117 | 30% | 12% | KRAS G12D (35G > A) | NOT APPLICABLE |
| NA09-128 | 80% | 45% (KRAS G12D) | KRAS G12D (35G > A) | NOT APPLICABLE |
| | | 44% (TP53 R248Q) | TP53 R248Q (743G > A) | |
| NA09-135 | 80% | 15% | KRAS G12V (35G > T) | NOT APPLICABLE |
| NA09-193 | N/A | 31% | KRAS G12V (35G > T) | NOT APPLICABLE |
| NA09-220 | 50% | 9% | KRAS G12V (35G > T) | NOT APPLICABLE |
| NA09-232 | 20-30% | 12% | KRAS G12V (35G > T) | NOT APPLICABLE |
| NA09-291 | N/A | 7% | KRAS G13D (38G > A) | NOT APPLICABLE |
| NA09-164 | 50% | 10% | TP53 R273L (818G > T) | NOT APPLICABLE |

N/A: not available
*Extremely limited tumor tissue

TABLE 7A
Amplification Primers

| Amplification | | SEQ ID |
|---|---|---|
| primer name | Sequence | NO: |
| APC_exon 16A_a1 | ACGTTGGATGAGCCAATGGT | 33 |
| | TCAGAAACAAA | |
| APC_exon 16A_a2 | ACGTTGGATGTGACACAAAG | 34 |
| | ACTGGCTTACA | |
| APC_exon 16B_a1 | ACGTTGGATGAGCAGTGTCA | 35 |
| | CAGCACCCTA | |
| APC_exon 16B_a2 | ACGTTGGATGCTTTGTGCCT | 36 |
| | GGCTGATTCT | |
| APC_exon 16C_a1 | ACGTTGGATGTCCTCAAACA | 37 |
| | GCTCAAACCA | |
| APC_exon 16C_a2 | ACGTTGGATGGCAGCATTTA | 38 |
| | CTGCAGCTTG | |
| APC_exon 16D_a1 | ACGTTGGATGCCAAGAGAAA | 39 |
| | GAGGCAGAAA | |
| APC_exon 16D_a2 | ACGTTGGATGTGTTGGCATG | 40 |
| | GCAGAAATAA | |
| BRAF_exon 15_a1 | ACGTTGGATGTGCTTGCTCT | 41 |
| | GATAGGAAAATG | |
| BRAF_exon 15_a2 | ACGTTGGATGCTGATGGGAC | 42 |
| | CCACTCCAT | |
| CTNNB1_exon 3_a1 | ACGTTGGATGTCACTGGCAG | 43 |
| | CAACAGTCTT | |
| CTNNB1_exon 3_a2 | ACGTTGGATGCAGGATTGCC | 44 |
| | TTTACCACTCA | |
| EGFR_exon 18_a1 | ACGTTGGATGCCAACCAAGC | 45 |
| | TCTCTTGAGG | |
| EGFR_exon 18_a2 | ACGTTGGATGcCTTATACAC | 46 |
| | CGTGCCGAAC | |
| EGFR_exon 19_a1 | ACGTTGGATGTCGAGGATTT | 47 |
| | CCTTGTTGGC | |
| EGFR_exon 19_a2 | ACGTTGGATGGATCCCAGAA | 48 |
| | GGTGAGAAAG | |
| EGFR_exon 20_a1 | ACGTTGGATGTGTTCCCGGA | 49 |
| | CATAGTCCAG | |
| EGFR_exon 20_a2 | ACGTTGGATGATCTGCCTCA | 50 |
| | CCTCCACCGT | |
| EGFR_exon 21_a1 | ACGTTGGATGCCTCCTTCTG | 51 |
| | CATGGTATTC | |
| EGFR_exon 21_a2 | ACGTTGGATGGCAGCATGTC | 52 |
| | AAGATCACAG | |
| FLT3_exon 20_a1 | ACGTTGGATGCACGGGAAAG | 53 |
| | TGGTGAAGAT | |
| FLT3_exon 20_a2 | ACGTTGGATGcATTGCCCCT | 54 |
| | GACAACATAG | |
| JAK2_exon 14_a1 | ACGTTGGATGAGCTTTCTCA | 55 |
| | CAAGCATTTGG | |
| JAK2_exon 14_a2 | ACGTTGGATGgctctgagaa | 56 |
| | aggcattagaa | |
| KIT_exon 17_a1 | ACGTTGGATGTCATGGTCGG | 57 |
| | ATCACAAAGA | |
| KIT_exon 17_a2 | ACGTTGGATGgagaatgggt | 58 |
| | actcacGTTTCC | |
| KRAS_exon 2_a1 | ACGTTGGATGtcattatttt | 59 |
| | tattataagGCCTGCTG | |
| KRAS_exon 2_a2 | ACGTTGGATGagaatggtcc | 60 |
| | tgcaccagtaa | |
| NOTCH1_exon 26A_a1 | ACGTTGGATGGGAGCATGTA | 61 |
| | CCCGAGAGG | |
| NOTCH1_exon 26A_a2 | ACGTTGGATGGAAGTGGAAG | 62 |
| | GAGCTGTTGC | |
| NOTCH1_exon 26B_a1 | ACGTTGGATGCAACAGCTCC | 63 |
| | TTCCACTTCC | |
| NOTCH1_exon 26B_a2 | ACGTTGGATGATCATCTGCT | 64 |
| | GGCCGTGT | |
| NRAS_exon 2_a1 | ACGTTGGATGcaacagGTTC | 65 |
| | TTGCTGGTGT | |
| NRAS_exon 2_a2 | ACGTTGGATGgagagacagg | 66 |
| | atcaggtcagc | |
| NRAS_exon 3_a1 | ACGTTGGATGTGGTGAAACC | 67 |
| | TGTTTGTTGG | |
| NRAS_exon 3_a2 | ACGTTGGATGcctttcagag | 68 |
| | aaaataatgctcct | |
| PIK3CA_exon 2_a1 | ACGTTGGATGCCCCTCCATC | 69 |
| | AACTTCTTCA | |
| PIK3CA_exon 2_a2 | ACGTTGGATGAAAAGCCGAA | 70 |
| | GGTCACAAAG | |
| PIK3CA_exon 10_a1 | ACGTTGGATGGACAAAGAAC | 71 |
| | AGCTCAAAGCAA | |
| PIK3CA_exon 10_a2 | ACGTTGGATGTTTAGCACTT | 72 |
| | ACCTGTGACTCCA | |
| PIK3CA_exon 21_a1 | ACGTTGGATGGAGCAAGAGG | 73 |
| | CTTTGGAGTA | |
| PIK3CA_exon 21_a2 | ACGTTGGATGATCCAATCCA | 74 |
| | TTTTTGTTGTCC | |
| PTEN_exon 5_a1 | ACGTTGGATGcttattctga | 75 |
| | ggttatctttttaccac | |
| PTEN_exon 5_a2 | ACGTTGGATGTGCACATATC | 76 |
| | ATTACACCAGTTC | |
| PTEN_exon 6_a1 | ACGTTGGATGttttctgtcc | 77 |
| | accagGGAGT | |
| PTEN_exon 6_a2 | ACGTTGGATGTCCAGATGAT | 78 |
| | TCTTTAACAGGTAGC | |
| PTEN_exon 7_a1 | ACGTTGGATGGGTGAAGATA | 79 |
| | TATTCCTCCAATTCA | |
| PTEN_exon 7_a2 | ACGTTGGATGttctcccaat | 80 |
| | gaaagtaaagtacaaa | |
| TP53_exon 5_a1 | ACGTTGGATGCAAGCAGTCA | 81 |
| | CAGCACATGA | |
| TP53_exon 5_a2 | ACGTTGGATGCTGCTCACCA | 82 |
| | TCGCTATCTG | |
| TP53_exon 7_a1 | ACGTTGGATGTGGCTCTGAC | 83 |
| | TGTACCACCA | |
| TP53_exon 7_a2 | ACGTTGGATGCCAGTGTGAT | 84 |
| | GATGGTGAGG | |
| TP53_exon 8_a1 | ACGTTGGATGCTACTGGGAC | 85 |
| | GGAACAGCTT | |
| TP53_exon 8_a2 | ACGTTGGATGGCTTCTTGTC | 86 |
| | CTGCTTGCTT | |
| ssIDH1_ex4_a1 | ACGTTGGATGGGCTTGTGAG | 87 |
| | TGGATGGGTA | |
| ssIDH1_ex4_a2 | ACGTTGGATGgcaaaatcac | 88 |
| | attattgccaac | |
| ssAKT1_ex3_a1 | ACGTTGGATGggtagagtgt | 89 |
| | gcgtggctct | |
| ssAKT1_ex3_a2 | ACGTTGGATGAGGTGCCATC | 90 |
| | ATTCTTGAGG | |
| ssBRAF_ex11_a1 | ACGTTGGATGtctgtttggc | 91 |
| | ttgacttgactt | |
| ssBRAF_ex11_a2 | ACGTTGGATGtcaccacatt | 92 |
| | acatacttacCATGC | |
| ssKRAS_ex3_a1 | ACGTTGGATGGTTTCTCCCT | 93 |
| | TCTCAGGATTC | |
| ssKRAS_ex3_a2 | ACGTTGGATGCCCACCTATA | 94 |
| | ATGGTGAATATCTTC | |
| ssMAP2K1_ex2_a1 | ACGTTGGATGattgacttgtg | 95 |
| | ctccccact | |
| ssMAP2K1_ex2_a2 | ACGTTGGATGCCCCAGCTCAC | 96 |
| | TGATCTTCT | |

TABLE 7B
Extension Primers

| Extension | | SEQ ID |
|---|---|---|
| primer name | Sequence | NO: |
| APC3340_extF | ACTGACTGACTGACTGACTGACTGACTGACTGACTGACTGACTG | 97 |
| | ACTGACTGACTGACTGATCCCAATGGTTCAGAAACAAAT | |
| APC4012_extF | TGACTGACTGACTGACTGACTGACTGACTGACTGACTGACTGAC | 98 |
| | TGACTGACTGACTGACTGATCACCAAATCCAGCAGACTG | |
| APC4348_extR | GACTGACTGACTGACTGACTGACTGACTGACTGACTGACTGACT | 99 |
| | GACTGATCGTGCTTTATTTTTAGGTACTTCTC | |
| APC4666_67insA_extF | GACTGACTGACTGACTGACTGACTGACTGACTGACTGACTGACT | 100 |
| | GACTGACTGATCAGAGAAAGAGGCAGAAAAAA | |
| BRAF1798_extF | TGACTGACTGACTGACTGACTGACTGACTGACTGGTGATTTTGG | 101 |
| | TCTAGCTACA | |
| BRAF1799_extF | GACTGACTGACTGACTGACTGACTGTGATTTTGGTCTAGCTACA | 102 |
| | G | |
| CTNNB1_94_extF | GACTGACTGACTGACTGACTGACTGACTGACTGACTGACTGACT | 103 |
| | GACTGACTGACTGCAGCAACAGTCTTACCTG | |
| CTNNB1_95_extR | CTGACTGACTGACTGACTGACTGACTGACTGACTGACTGACTGA | 104 |
| | CTGACTGACTGAACCAGAATGGATTCCAGAG | |
| CTNNB1_98_extF | GACTGGCAACAGTCTTACCTGGACT | 105 |
| CTNNB1_101_extF | ACTGACTGACTGACTGATAACAGTCTTACCTGGACTCTG | 106 |
| CTNNB1_109_extF | CTGACTGACTGACTGACTGACTGACTGCTGGACTCTGGAATCCA | 107 |
| | T | |
| CTNNB1_110_extF | CTGACTGTGGACTCTGGAATCCATT | 108 |
| CTNNB1_121_extR | CTGACTGACTGAcTGACTGACTGACTGACTGACTGACTGAcTGA | 109 |
| | CTGACTGACTGACTGACTGACTGACAGAGAAGGAGCTGTGG | |
| CTNNB1_122_extR | GACTGACTGAcTGACTGACTGACTGACTGACTGACTGAcTGACT | 110 |
| | GACTGACTGACTGACTGACTGAcCTCAGAGAAGGAGCTGTG | |
| CTNNB1_133_extR | GACTGACTGACTGACTGACTGACTGACTGACTGACTGTGCCTTT | 111 |
| | ACCACTCAGAG | |
| CTNNB1_134_extR | CTGACTGACTGACTGACTGACTGACTGTTGCCTTTACCACTCAG | 112 |
| | A | |
| EGFR2155_extF | GACTGACTGACTGACTGACTGACTGTCAAAAAGATCAAAGTGCT | 113 |
| | G | |
| EGFR2235_2249 | CTGACTGACTGACTGACTGTTCCCGTCGCTATCAA | 114 |
| del_extF | | |
| EGFR2235_2249 | TGGCTTTCGGAGATGTT | 115 |
| del_extR | | |
| EGFR2236_2250 | CTGACTGACTGACTGACTGTCCCGTCGCTATCAAG | 116 |
| del_extF | | |
| EGFR2236_2250 | GACTGACTGACTGACTGATTGGCTTTCGGAGATGT | 117 |
| del_extR | | |
| EGFR2369_extR | CTGACTGACTGACTGACTGACTGACTGACTAAGGGCATGAGCTG | 118 |
| | C | |
| EGFR2573_extF | GACTGACTGACTGACTGACTGACTGACAGATCACAGATTTTGGG | 119 |
| | C | |
| FLT3_2503_extF | GACTGACTGACTGACTGACTGACTGACTGACTGACTGACTGACT | 120 |
| | GACTGACTACTTTGGATTGGCTCGA | |
| JAK2_1849_extF | ACTGACTGACTGACTGACTGTTTGGTTTTAAATTATGGAGTATG | 121 |
| | T | |
| KIT2447_extF | GACTGACTGACTGACTGACTGACTGACTGACTGACGATTTTGGT | 122 |
| | CTAGCCAGAG | |
| KRAS34_extR | GACTGAcTGCTCTTGCCTACGCCAC | 123 |
| KRAS35_extF | CTGACtCTTGTGGTAGTTGGAGCTG | 124 |
| KRAS37_extF | TGACTGACtGATGGTAGTTGGAGCTGGT | 125 |
| KRAS38_extF | GACTGACTGACGGTAGTTGGAGCTGGTG | 126 |
| NOTCH1_4724_extR | CTGACTGACTGACTGACTGACTGACTGACTGACTGACTGACTGA | 127 |
| | CTGACTGACTGACTGACTGACTGACGCACCACCACCACC | |
| NOTCH1_4802_extF | ACTGACTGACTGACTGACTGACTGACTGACTGACTGACTGACTG | 128 |
| | ACTGACTGACTGACTGACTGACTGACTGACTGACTCAGCCGCGT | |
| | GC | |
| NRAS34_extR | GACTGACTGCTTTTCCCAACACCAC | 129 |
| NRAS35_extF | CTGACTGACTGACTGACTGACTGACTGACTGACTGACTGACTGA | 130 |
| | CTGACTGACTGAGTGGTGGTTGGAGCAG | |
| NRAS37_extR | GACTGACTGACTGACTGACTGACTGACTGACTGACTGACTGACT | 131 |
| | GACTCGCTTTTCCCAACAC | |
| NRAS38_extR | ACTGACTGACTGACTGACTGGCGCTTTTCCCAACA | 132 |
| NRAS181_extF | GACTGACTGACTGACTGACTGACTGACTGACTGACACATACTGG | 133 |
| | ATACAGCTGGA | |
| NRAS182_extF | CTGACTGACTGACTGACTGACTGACTGACTGACTGCATACTGGA | 134 |
| | TACAGCTGGAC | |
| NRAS183_extR | GACTGACTGACTGACTGACTGACTGACTGACTGACTGACTGACT | 135 |
| | GACTGCTCATGGCACTGTACTCTTC | |
| PIK3CA263_extF | CTGACTGACTGACTGACTGACTGACTGACTGACTGACTAAGAAT | 136 |
| | TTTTTGATGAAACAAGAC | |
| PIK3CA1624_extR | TGACTGACTGACTGACTGACTGACTGACTGACTGACTGACTGAC | 137 |
| | TTCTCCTGCTCAGTGATTT | |
| PIK3CA1633_extF | GACTGACTGACTGACTGACTGACTGACTGACTGACTGACTGACT | 138 |
| | GATCCTCTCTCTGAAATCACT | |
| PIK3CA1636_extF | ACTGACTGACTGACTGACTGACTGACTGACTGACTGACTGACTG | 139 |
| | ACTGCCTCTCTCTGAAATCACTGAG | |
| PIK3CA1637_extF | ACTGACTGACTGACTGACTGACTGACTGACTGACTGACTGACTG | 140 |
| | ACTGCTCTCTCTGAAATCACTGAGC | |
| PIK3CA3139_extR | TGACTGACTGACTGACTGACTGACTGACTGACTGACTGACTGAC | 141 |
| | TGACTGACTGACTGACGTCCAGCCACCATGAT | |
| PIK3CA3140_extR | GTCCAGCCACCATGA | 142 |
| PIK3CA3145_extR | ACTGACTGACTGACTGACTGACTGACTGACTGACTGACTGACTG | 143 |
| | ATTTTGTTGTCCAGCCAC | |
| PTEN388_extF | TGACTGACTGACTGACTTGTAAAGCTGGAAAGGGA | 144 |
| PTEN517_extF | ACTGACTGACTGACTGACTGACTAGTAACTATTCCCAGTCAGAG | 145 |
| | G | |
| PTEN697_extR | ACTGACTGACTGACTGACTGACTGACTGACTGACTGTGAACTTG | 146 |
| | TCTTCCCGTC | |
| PTEN800delA_extF | GACTGACTGACTGACTGACTGACTGACTGACTGACTGACTAAAC | 147 |
| | AGAACAAGATGCTAAAAA | |
| TP53_524_extF | GACTGACTGACTGACTGACTGACTGACTGACTGACTGACTGACT | 148 |
| | GACTGACTGACTGACTGACTGACTGACTGACCGGAGGTTGTGAG | |
| | GC | |
| TP53_733_extR | GACTGACTGACTGACTGACTGACTGACTGACTGACTGACTCCTC | 149 |
| | CGGTTCATGC | |
| TP53_742_extF | ACTGACTGACTGACTGACTGACTGACTGACTGACTGACTGACTG | 150 |
| | ACTGACTGACTGGGGCGGCATGAAC | |
| TP53_743_extF | CTGACTGACTGACTGACTGACTGACTGACTGACTGACTGACGGC | 151 |
| | GGCATGAACC | |
| TP53_817_extF | CTGACTGACTGACTGACTGACTGACTGACTGACTGACTGACTGA | 152 |
| | CTGACTGAGGAACAGCTTTGAGGTG | |
| TP53_818_extF | ACTGACTGACTGACTGACTGACTGACTGACTGACTGACTGACTG | 153 |
| | ACTGACTGACTGACTGACTGACTGACTGAGAACAGCTTTGAGGT | |
| | GC | |
| TP53_916_extR | TGACTGACTGACTGACTGACTGACTGACTGACTGACTGACTGAC | 154 |
| | TGACTGACTGACTGACTGACTGACTGACTGACTGAGTCCTGCTT | |
| | GCTTACCTC | |
| ssIDH1.395_extR* | TGATCCCCATAAGCATGA | 155 |
| ssIDH1.394_extR* | GACTGACTGGACTGACTGACTGACTGACTGGACTGACTGACTGA | 156 |
| | GATCCCCATAAGCATGAC | |
| ssAKT1.49_extR | CTGACTGACTGACTGACTGACTGACTGACTGACTGACTGACTGA | 157 |
| | CTGACTGACTGACTGACTGACTGACTGAGCCAGGTCTTGATGTA | |
| | CT | |
| ssBRAF1397_extF | GACTGACTGACTGACTGACTGACTGACTGACTGACTGACTGACT | 158 |
| | GACTGACTGACTGACTGACTGGACAAAGAATTGGATCTG | |
| ssBRAF1406_extR | TGACTGACTGACTGACTGACTGACTGACTGACTGACTGACTGAC | 159 |
| | TGACTGACTGACTGACTGACTGACTCACTTTCCCTTGTAGACTG | |
| | TT | |
| ssBRAF1789_extF | GACTGACTACAGTAAAAATAGGTGATTTTGGT | 160 |
| ssEGFR_2582_extR* | GACTGACTGACTGACTGACTGACTGACTGACTGACTGACTGACT | 161 |
| | GACTGACTGACTGACTGACTGACTGCTTCCGCACCCAGC | |
| ssEGFR_2156_extF** | ACTGACTGACTGACTGACTGACTGACTGACTGACTGACTGACCA | 162 |
| | AAAAGATCAAAGTGCTGG | |
| ssKRAS181_extF | ATTCTCGACACAGCAGGT | 163 |
| ssKRAS182_extF | ACTGACTGACTGACTGACTGACTGACTGACTGACTGACTGACTG | 164 |
| | ATCTCGACACAGCAGGTC | |
| ssKRAS183_extR* | TGACTGACTGACTGACTGACTGACTGACTGACTGCTCATTGCAC | 165 |
| | TGTACTCCTC | |
| ssMAP2K1.167_extR* | TGACTGACTGACTGACTGACTGACTGACTGACTGACTGACTGAC | 166 |
| | TGACTGACTGACTGACTCCCACCTTCTGCTTC | |
| ssMAR2K1.171_extR | CTGACTGACTGACTGACTGACTGACTGACTGACTGACTGACTGA | 167 |
| | CTGACTGACTGACCAGTTCTCCCACCTTCTG | |
| ssMAP2K1.199_extR | ACTGACTGACTGACTGACTGACTGACTGACTGACTGACTGACTG | 168 |
| | ACTGACTGACTGACTGGCTCACTGATCTTCTCAAAGT | |

TABLE 8A
PCR Primer Mixes

| | [Stock] | Volume |
|---|---|---|
| Panel I - PCR Primers (F + R) | | |
| 4X KRAS exon 2 | 3 μM | 400 μl |
| 2X EGFR exon 19 | 3 μM | 200 μl |
| EGFR exon 20 | 3 μM | 100 μl |
| 2X NRAS exon 3 | 3 μM | 200 μl |
| PI3K exon 10 | 3 μM | 100 μl |
| 2X β-Cat exon 3 | 3 μM | 200 μl |
| Nuclease-Free dH₂O | — | 200 μl |
| Panel II - PCR Primers (F + R) | | |
| EGFR exon 19 | 3 μM | 100 μl |
| NRAS exon 2 | 3 μM | 100 μl |
| BRAF exon 15 | 3 μM | 100 μl |
| NRAS exon 3 | 3 μM | 100 μl |
| PI3K exon 2 | 3 μM | 100 μl |
| TP53 exon 7 | 3 μM | 100 μl |
| 2X β-Cat exon 3 | 3 μM | 200 μl |
| Nuclease-Free dH₂O | — | 600 μl |
| Panel III - PCR Primers (F + R) | | |
| EGFR exon 19 | 3 μM | 100 μl |
| NRAS exon 2 | 3 μM | 100 μl |
| EGFR exon 21 | 3 μM | 100 μl |
| β-Cat exon 3 | 3 μM | 100 μl |
| PI3K exon 10 | 3 μM | 100 μl |
| AKT1 exon 3 | 3 μM | 100 μl |
| IDH1 exon 4 | 3 μM | 100 μl |
| Nuclease-Free dH2O | — | 700 μl |
| Panel IV - PCR Primers (F + R) | | |
| 2X KRAS exon 2 | 3 μM | 200 μl |
| EGFR exon 19 | 3 μM | 100 μl |
| PTEN exon 6 | 3 μM | 100 μl |
| TP53 exon 7 | 3 μM | 100 μl |
| PI3K exon 21 | 3 μM | 100 μl |
| NOTCH exon 26A | 3 μM | 100 μl |
| NOTCH exon 26B | 3 μM | 100 μl |
| IDH1 exon 4 | 3 μM | 100 μl |
| Nuclease-Free dH₂O | — | 500 μl |
| Panel V - PCR Primers (F + R) | | |
| 2X b-cat exon 3 | 3 μM | 200 μl |
| 2X KRAS exon 2 | 3 μM | 200 μl |
| TP53 exon 7 | 3 μM | 100 μl |
| TP53 exon 8 | 3 μM | 100 μl |
| 2X APC exon 16D | 3 μM | 200 μl |
| BRAF exon 11 | 3 μM | 100 μl |
| KRAS exon 3 | 3 μM | 100 μl |
| Nuclease-Free dH₂O | — | 400 μl |
| Panel VI - PCR Primers (F + R) | | |
| β-Cat exon 3 | 3 μM | 100 μl |
| 2X KRAS exon 2 | 3 μM | 200 μl |
| EGFR exon 18 | 3 μM | 100 μl |
| KIT exon 17 | 3 μM | 100 μl |
| PI3K exon 21 | 3 μM | 100 μl |
| PI3K ex 10 | 3 μM | 100 μl |
| MAP2K1 exon 2 | 3 μM | 100 μl |
| APC exon 16 B | 3 μM | 100 μl |
| 2X TP53 exon 8 | 3 μM | 200 μl |
| Nuclease-Free dH₂O | — | 300 μl |
| Panel VII - PCR Primers (F + R) | | |
| 2X PI3K exon 21 | 3 μM | 200 μl |
| β-Cat exon 3 | 3 μM | 100 μl |
| 2X BRAF exon 15 | 3 μM | 200 μl |
| 2X NRAS exon 2 | 3 μM | 200 μl |
| PI3K ex 10 | 3 μM | 100 μl |
| 2X APC exon 16 C | 3 μM | 200 μl |
| 2X APC exon 16 A | 3 μM | 200 μl |
| Nuclease-Free dH₂O | — | 200 μl |
| Panel VIII - PCR Primers (F + R) | | |
| 2X NRAS exon 2 | 3 μM | 200 μl |
| 2X PTEN exon 5 | 3 μM | 200 μl |
| β-Cat exon 3 | 3 μM | 100 μl |
| 2X PTEN exon 7 | 3 μM | 200 μl |
| NRAS exon 3 | 3 μM | 100 μl |
| 2X TP53 exon 5 | 3 μM | 200 μl |
| TP53 exon 8 | 3 μM | 100 μl |
| Nuclease-Free dH₂O | — | 300 μl |
| Panel IX - PCR Primers (F + R) | | |
| MAP2K1 exon 2 | 3 μM | 100 μl |
| KRAS exon 3 | 3 μM | 100 μl |
| BRAF exon 15 | 3 μM | 100 μl |
| EGFR exon 18 | 3 μM | 100 μl |
| Nuclease-Free dH₂O | — | 1000 μl |

TABLE 8B
Extension Primer Mixes

| | Primer | Volume |
|---|---|---|
| | Stock | (8 μl Total) |
| PANEL I | | |
| Extension Primers | | |
| KRAS34_R | 10 μM | 0.8 μl |
| EGFR2235_49 del#1_F | 5 μM | 0.9 μl |
| EGFR2369_R | 10 μM | 1.0 μl |
| NRAS181 F | 10 μM | 1.3 μl |
| PI3K 1633_F | 2 μM | 0.6 μl |
| b-cat 94_F | 5 μM | 0.3 μl |
| b-cat121_R | 5 μM | 1.0 μl |
| Nuclease-Free dH₂O | — | 2.1 μl |
| PANEL II | | |
| Extension Primers | | |
| EGFR2235_49del#2_R* | 2 μM | 0.4 μl |
| NRAS38_R* | 10 μM | 1.5 μl |
| BRAF1799_F | 2 μM | 1.0 μl |
| NRAS182_F | 2 μM | 0.5 μl |
| PI3K263_F* | 10 μM | 0.3 μl |
| TP53.742_extF* | 10 μM | 1.0 μl |
| b-cat95_R* | 5 μM | 0.7 μl |
| b-cat122_R | 5 μM | 0.4 μl |
| Nuclease-Free dH₂O | — | 2.2 μl |
| PANEL III | | |
| Extension Primers | | |
| EGFR2236_50del#1_F | 5 μM | 0.5 μl |
| EGFR2573_F | 10 μM | 0.5 μl |
| b-cat133_R | 5 μM | 0.5 μl |
| PI3K1624_R* | 10 μM | 1.0 μl |
| NRAS35_F | 5 μM | 0.3 μl |
| AKT1.49_extR | 10 μM | 1.2 μl |
| IDH1.395_extR | 10 μM | 0.6 μl |
| EGFR2582_extR* | 10 μM | 0.4 μl |
| Nuclease-Free dH₂O | — | 3 μl |
| PANEL IV | | |
| Extension Primers | | |
| KRAS35_F | 5 μM | 0.2 μl |
| EGFR2236_50del#2_R | 5 μM | 0.2 μl |
| PTEN517_F | 5 μM | 1.2 μl |
| TP53.733_R* | 50 μM | 0.5 μl |
| PI3K3139_R | 2 μM | 0.3 μl |
| NOTCH1.4724_R | 50 μM | 0.5 μl |
| NOTCH1.4802_F | 50 μM | 1.0 μl |
| IDH1.394_extR* | 10 μM | 0.3 |
| Nuclease-Free dH₂O | — | 3.8 μl |
| PANEL V | | |
| Extension Primers | | |
| b-cat110_F | 2 μM | 1.5 μl |
| KRAS38_F* | 5 μM | 0.7 μl |
| b-cat134_R | 2 μM | 0.9 μl |
| TP53.743_F* | 10 μM | 1.0 μl |
| TP53.817_F* | 10 μM | 1.0 μl |
| APC4666_67insA_F | 10 μM | 1.8 μl |
| BRAF1397_extF | 10 μM | 0.3 μl |
| BRAF1406_extR | 10 μM | 0.3 μl |
| KRAS182_extF | 10 μM | 0.3 μl |
| Nuclease-Free dH₂O | — | 0.2 μl |
| PANEL VI | | |
| Extension Primers | | |
| b-cat98_F | 2 μM | 0.6 μl |
| KRAS37_F* | 5 μM | 0.2 μl |
| EGFR2155_F | 10 μM | 0.3 μl |
| KIT2447_F | 10 μM | 1.2 μl |
| PI3K3145_R | 10 μM | 1.0 μl |
| PI3K1637_F | 2 μM | 1.0 μl |
| MAP2K1.167_extR* | 10 μM | 0.3 μl |
| APC4012_F | 10 μM | 1.5 μl |
| TP53.818_F | 10 μM | 0.6 μl |
| Nuclease-Free dH₂O | — | 1.3 μl |
| PANEL VII | | |
| Extension Primers | | |
| PI3K3140_R | 2 μM | 1.4 μl |
| b-cat101_F* | 2 μM | 0.6 μl |
| BRAF1798_F | 2 μM | 0.7 μl |
| NRAS37_R | 10 μM | 2.4 μl |
| PI3K1636_F | 10 μM | 0.8 μl |
| APC4348_R* | 10 μM | 0.1 μl |
| APC3340_F | 10 μM | 0.7 μl |
| Nuclease-Free dH₂O | — | 1.3 μl |
| PANEL VIII | | |
| Extension Primers | | |
| NRAS34_R | 10 μM | 0.4 μl |
| PTEN388_F | 10 μM | 2.0 μl |
| b-cat109_F | 10 μM | 0.5 μl |
| PTEN697_R | 10 μM | 0.5 μl |
| PTEN800delA_F | 10 μM | 1.0 μl |
| NRAS183_R* | 10 μM | 1.0 μl |
| TP53.524_F | 10 μM | 2.0 μl |
| TP53.916_R | 5 μM | 0.4 μl |
| Nuclease-Free dH₂O | — | 0.2 μl |
| PANEL IX | | |
| Extension Primers | | |
| BRAF1789_extF | 10 μM | 0.3 μl |
| KRAS181_extF | 10 μM | 0.3 μl |
| BRAF1799_extR | 2 μM | 0.3 μl |
| MAP2K1.171_extR | 10 μM | 0.8 μl |
| MAP2K1.199_extR | 10 μM | 0.6 μl |
| KRAS183_extR | 10 μM | 0.3 μl |
| EGFR2156_extF | 10 μM | 0.3 μl |
| Nuclease-Free dH₂O | — | 5.1 μl |

TABLE 9
Synthetic oligonucleotides used for assay validation

| Oligonucleotide name¹ | Sequence | Amount² | SEQ ID NO: |
|---|---|---|---|
| A.ctrl_APC4348C > T | GTACTTCTCACTTGGTTTGAGCTGTTTGAGAAAAA | 40 pmol | 169 |
| A.ctrl_APC4666_67 insA | AATCAATAGTTTTTTTCTGCCTCTTTCTCTAAAAA | 3 pmol | 170 |
| A.ctrl_BRAF1798G > A | GAGATTTCATTGTAGCTAGACCAAAATCACAAAAA | 3 pmol | 171 |
| A.ctrl_CTNNB1_94G > A | ATTCCAGAGTTCAGGTAAGACTGTTGCTGCAAAAA | 3 pmol | 172 |
| A.ctrl_CTNNB1_94G > C | ATTCCAGAGTGCAGGTAAGACTGTTGCTGCAAAAA | 3 pmol | 173 |
| A.ctrl_CTNNB1_94G > T | ATTCCAGAGTACAGGTAAGACTGTTGCTGCAAAAA | 3 pmol | 174 |
| S.ctrl_CTNNB1_95A > G | GCAGCAACAGTCTTACCTGGGCTCTGGAATCCATTCTGGT | 30 pmol | 175 |
| A.ctrl_CTNNB1_98C > G | ATGGATTCCACAGTCCAGGTAAGACTGTTGAAAAA | 3 pmol | 176 |
| A.ctrl_CTNNB1_101G > A | ATGGATTTCAGAGTCCAGGTAAGACTGTTGAAAAA | 3 pmol | 177 |
| A.ctrl_CTNNB1_101G > T | ATGGATTACAGAGTCCAGGTAAGACTGTTGAAAAA | 10 pmol | 178 |
| A.ctrl_CTNNB1_109T > G | GTGGCACCAGCATGGATTCCAGAGTCCAGGAAAAA | 3 pmol | 179 |
| A.ctrl_CTNNB1_110C > A | GTGGCACCATAATGGATTCCAGAGTCCAGGAAAAA | 3 pmol | 180 |
| A.ctrl_CTNNB1_110C > G | GTGGCACCACAATGGATTCCAGAGTCCAGGAAAAA | 3 pmol | 181 |
| A.ctrl_CTNNB1_110C > T | GTGGCACCAAAATGGATTCCAGAGTCCAGGAAAAA | 3 pmol | 182 |
| S.ctrl_CTNNB1_122C > T | AATCCATTCTGGTGCCACTATCACAGCTCCTTCTCTGAGT | 30 pmol | 183 |
| S.ctrl_CTNNB1_133T > C | TGCCACTACCACAGCTCCTCCTCTGAGTGGTAAAGGCAAT | 3 pmol | 184 |
| A.ctrl_EGFR2155G > T | GCACCGGAGCACAGCACTTTGATCTTTTTGAAAAA | 3 pmol | 185 |
| A.ctrl_KIT2447A > T | TTCTTGATGACTCTGGCTAGACCAAAATCAAAAAA | 3 pmol | 186 |
| A.ctrl_KRAS35G > C | CCTACGCCAGCAGCTCCAACTACCACAAGTAAAAA | 10 pmol | 187 |
| A.ctrl_KRAS37G > T | TCTTGCCTACGCAACCAGCTCCAACTACCAAAAAA | 3 pmol | 188 |
| S.ctrl_NOTCH1_4724T > C | GAGGCTGGCGGCCGGCACGCCGGTGGTGGTGGTGCTGATG | 1 pmol | 189 |
| A.ctrl_NOTCH1_4802T > C | GAAGACCACGTTGGTGTGCGGCACGCGGCTGAGCTCCCGC | 3 pmol | 190 |
| S.ctrl_NRAS34G > A | ACTGGTGGTGGTTGGAGCAAGTGGTGTTGGGAAAAGCGCA | 3 pmol | 191 |
| S.ctrl_NRAS35G > C | ACTGGTGGTGGTTGGAGCAGCTGGTGTTGGGAAAAGCGCA | 3 pmol | 192 |
| A.ctrl_NRAS35G > C | TGCGCTTTTCCCAACACCAGCTGCTCCAACCACCACCAGT | 3 pmol | 193 |
| S.ctrl_NRAS37G > T | GGTGGTGGTTGGAGCAGGTTGTGTTGGGAAAAGCGCACTG | 1 pmol | 194 |
| S.ctrl_NRAS38G > A | GGTGGTGGTTGGAGCAGGTGATGTTGGGAAAAGCGCACTG | 3 pmol | 195 |
| S.ctrl_NRAS38G > T | GGTGGTGGTTGGAGCAGGTGTTGTTGGGAAAAGCGCACTG | 2 pmol | 196 |
| A.ctrl_NRAS182A > C | TACTCTTCTGGTCCAGCTGTATCCAGTATGAAAAA | 5 pmol | 197 |
| A.ctrl_NRAS182A > G | TACTCTTCTCGTCCAGCTGTATCCAGTATGAAAAA | 3 pmol | 198 |
| S.ctrl_NRAS183A > C | CATACTGGATACAGCTGGACACGAAGAGTACAGTGCCATG | 3 pmol | 199 |
| S.ctrl_NRAS183A > T | CATACTGGATACAGCTGGACATGAAGAGTACAGTGCCATG | 3 pmol | 200 |
| A.ctrl_PIK3CA1636C > A | TCTTTCTCCTTCTCAGTGATTTCAGAGAGAAAAAA | 3 pmol | 201 |
| S.ctrl_PIK3CA3140A > T | AAACAAATGAATGATGCACTTCATGGTGGCTGGACAACAA | 3 pmol | 202 |
| S.ctrl_PIK3CA3145G > A | AATGAATGATGCACATCATAGTGGCTGGACAACAAAAATG | 10 pmol | 203 |
| A.ctrl_PTEN388C > G | ACACCAGTTCCTCCCTTTCCAGCTTTACAGAAAAA | 1 pmol | 204 |
| A.ctrl_PTEN388C > T | ACACCAGTTCATCCCTTTCCAGCTTTACAGAAAAA | 3 pmol | 205 |
| S.ctrl_TP53_733G > A | TAACAGTTCCTGCATGGGCAGCATGAACCGGAGGCCCATC | 3 pmol | 206 |
| A.ctrl_TP53_817C > T | GCACAAACACACACCTCAAAGCTGTTCCGTAAAAA | 3 pmol | 207 |

¹S.ctrl primers were designed as the coding strand (sense) for validation of reverse orientation assays.
A.ctrl primers were designed as the non-coding strand (antisense) for validation of forward orientation assays.
²Amount of mutation control oligonucleotide used for SNaPshot assay validation.

TABLE 10
Sequencing Primers

| Sequencing | | SEQ | An- | |
|---|---|---|---|---|
| Primer | | ID | nealing | |
| Name | Sequence | NO: | Temp | MgCl₂ |
| APC_ex16A_ | TGTAAAACGACGGCCAGTGAG | 208 | 58° C. | 40 |
| Seq_a1 ^{M13} | CACTGATGATAAACACCTCAA | | | nmol |
| APC_ex16A_ | CAGGAAACAGCTATGACCATA | 209 | | |
| Seq_a2 ^{M13} | GGCTGATCCACATGACGTT | | | |
| CTNNB1_ex | GATTTGATGGAGTTGGACATG | 210 | 60° C. | 50 |
| 3_Seq_a1 | G | | | nmol |
| CTNNB1_ex | TGTTCTTGAGTGAAGGACTGA | 211 | | |
| 3_Seq_a2 | | | | |
| EGFR_ex18_ | TGTAAAACGACGGCCAGTCTG | 212 | 65° C. | 40 |
| Seq_a1 ^{M13} | AGGTGACCCTTGTCTCTG | | | nmol |
| EGFR_ex18_ | CAGGAAACAGCTATGACCTAC | 213 | | |
| Seq_a2 ^{M13} | AGCTTGCAAGGACTCTGG | | | |
| EGFR_ex19_ | TGTAAAACGACGGCCAGTGGT | 214 | 65° C. | 40 |
| Seq_a1 ^{M13} | AACATCCACCCAGATCAC | | | nmol |
| EGFR_ex19_ | CAGGAAACAGCTATGACCTGA | 215 | | |
| Seq_a2 ^{M13} | GCAGGGTCTAGAGCAGAG | | | |
| EGFR_ex20_ | TGTAAAACGACGGCCAGTCGA | 216 | 65° C. | 40 |
| Seq_a1 ^{M13} | AGCCACACTGACGTGC | | | nmol |
| EGFR_ex20_ | CAGGAAACAGCTATGACCCTC | 217 | | |
| Seq_a2 ^{M13} | CTTATCTCCCCTCCCCG | | | |
| EGFR_ex21_ | TGTAAAACGACGGCCAGTTCT | 218 | 65° C. | 40 |
| Seq_a1 ^{M13} | TCCCATGATGATCTGTCC | | | nmol |
| EGFR_ex21_ | CAGGAAACAGCTATGACCCCT | 219 | | |
| Seq_a2 ^{M13} | GGTGTCAGGAAAATGCT | | | |
| JAK2_ex12_ | GCAGCAAGTATGATGAGCAAG | 220 | 65° C. | 40 |
| Seq_a1 | CTTTC | | | nmol |
| JAK2_ex12_ | CAGATGCTCTGAGAAAGGCAT | 221 | | |
| Seq_a2 | TAG | | | |
| PIK3CA_ex21_ | TGTAAAACGACGGCCAGTCAT | 222 | 58° C. | 40 |
| Seq_a1 ^{M13} | ACATTCGAAAGACCCTAGCC | | | nmol |
| PIK3CA_ex21_ | CAGGAAACAGCTATGACCATG | 223 | | |
| Seq_a2 ^{M13} | GATTGTGCAATTCCTATGC | | | |
| TP53_ex5_ | CTTGTGCCCTGACTTTCAAC | 224 | 64° C. | 40 |
| Seq_a1 | | | | nmol |
| TP53_ex5_ | ACCAGCCCTGTCGTCTCTC | 225 | | |
| Seq_a2 | | | | |
| TP53_ex6_ | AGGCCTCTGATTCCTCACTG | 226 | 62° C. | 50 |
| Seq_a1 | | | | nmol |
| TP53_ex6_ | ACTGACAACCACCCTTAACC | 227 | | |
| Seq_a2 | | | | |
| TP53_ex7_ | TCATCTTGGGCCTGTGTTATC | 228 | 58° C. | 50 |
| Seq_a1 | | | | nmol |
| TP53_ex7_ | GAAATCGGTAAGAGGTGGGC | 229 | | |
| Seq_a2 | | | | |
| TP53_ex8_ | TTTCCTTACTGCCTCTTGCTT | 230 | 58° C. | 50 |
| Seq_a1 | C | | | nmol |
| TP53_ex8_ | GGAAAGGTGATAAAAGTGAAT | 231 | | |
| Seq_a2 | CTG | | | |
| M13_Seq_a1 | TGTAAAACGACGGCCAGT | 232 | N/A | N/A |
| M13_Seq_a2 | CAGGAAACAGCTATGACC | 233 | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of providing a genetic profile of a tumor, the method comprising:
providing a sample comprising genomic DNA from a tumor cell; and
simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, thereby providing a genetic profile of the tumor.

2. The method of claim 1, wherein the method comprises determining the identity of about 6 to 9 alleles in a single reaction.

3. The method of claim 1, wherein the method comprises determining the identity of all alleles listed in Table 3B.

4. The method of claim 3, wherein the method comprises performing a plurality of reactions as set forth in Tables 8A and 8B.

5. The method of claim 1, wherein the tumor cell is from a lung, breast, colorectal, head and neck, or ovarian tumor.

6. The method of claim 1, wherein the tumor cell is in a formalin-fixed paraffin-embedded biopsy sample.

7. A method of selecting an appropriate chemotherapy for a subject, the method comprising:
providing a sample comprising genomic DNA from a tumor cell from the subject;
simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, to provide a genetic profile of the tumor; and
selecting an appropriate chemotherapy based on the genetic profile of the tumor.

8. The method of claim 7, wherein the method comprises determining the identity of about 6 to 9 alleles in a single reaction.

9. The method of claim 7, wherein the method comprises determining the identity of all alleles listed in Table 3B.

10. The method of claim 9, wherein the method comprises performing a plurality of reactions as set forth in Tables 8A and 8B.

11. The method of claim 7, wherein if an EGFR 2369C>T, KRAS 34G>T, KRAS 34G>C, KRAS 34G>A, KRAS 35G>T, KRAS 35G>C, KRAS 35G>A, KRAS 37G>T, KRAS 37G>C, KRAS 37G>A, KRAS 38G>T, KRAS 38G>C, or KRAS 38G>A mutation is present, then a therapy comprising an EGFR inhibitor is not selected.

12. The method of claim 7, further comprising administering the selected chemotherapy to the subject.

13. The method of claim 12, wherein the chemotherapeutic agent is erlotinib or gefitinib.

14. The method of claim 7, wherein the subject has lung cancer, breast cancer, colorectal cancer, head and neck cancer, or ovarian cancer.

15. A method of determining a prognosis for a subject diagnosed with cancer, the method comprising:
providing a sample comprising genomic DNA from a tumor cell from the subject;
simultaneously determining the identity of one or more alleles listed in Table 3B for each of EGFR and KRAS plus one or more alleles from one or more of AKT1, APC, BRAF, CTNNB1, FLT3, IDH1, JAK2, KIT, MAP2K1, NOTCH1, NRAS, PIK3CA, PTEN, and TP53 in the genomic DNA, wherein the method comprises determining the identity of each allele using a single base extension reaction, to provide a genetic profile of the tumor; and
determining a prognosis for the subject based on the genetic profile of the tumor.

16. The method of claim 15, wherein the method comprises determining the identity of about 6 to 9 alleles in a single reaction.

17. The method of claim 15, wherein the method comprises determining the identity of all alleles listed in Table 3B.

18. The method of claim 17, wherein the method comprises performing a plurality of reactions as set forth in Tables 8A and 8B.

19. The method of claim 15, wherein the subject has a plurality of tumors and the method comprises determining the genetic profile of more than one tumor in the subject.

20. The method of claim 19, wherein the presence of an identical profile in each tumor indicates that the cancer is metastatic, and the presence of a different profile in each tumor indicates that the cancer is not metastatic.

21. The method of claim 15, wherein the subject has lung cancer, breast cancer, colorectal cancer, head and neck cancer, or ovarian cancer.

22. A kit comprising the primers listed in Table 7.

23. The kit of claim 22, wherein the primers are provided in a container in the combinations as listed in Tables 8A and 8B.


SEQUENCE LISTING (248 sequences)

SEQ ID NO: 1 - DNA, 1443 bp, Homo sapiens
  CDS (1)..(1440)
atgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctga

SEQ ID NO: 2 - protein, 480 aa, Homo sapiens [sequence omitted]

SEQ ID NO: 3 - DNA, 8532 bp, Homo sapiens
  CDS (1)..(8529)
atggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaa

SEQ ID NO: 4 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKDFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 5 - DNA, 2301 bp, Homo sapiens
  CDS (1)..(2298)
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 6 - protein, 766 aa, Homo sapiens [sequence omitted]

SEQ ID NO: 7 - DNA, 2346 bp, Homo sapiens
  CDS (1)..(2343)
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 8 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 9 - DNA, 3633 bp, Homo sapiens
  CDS (1)..(3630)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaagcttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 10 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 11 - DNA, 2982 bp, Homo sapiens
  CDS (1)..(2979)
atgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaacgtgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggatggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtag

SEQ ID NO: 12 - protein, 993 aa, Homo sapiens [sequence omitted]

SEQ ID NO: 13 - DNA, 1245 bp, Homo sapiens
  CDS (1)..(1242)
atgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaa

SEQ ID NO: 14 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 15 - DNA, 3399 bp, Homo sapiens
  CDS (1)..(3396)
atgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatga

SEQ ID NO: 16 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 17 - DNA, 2931 bp, Homo sapiens
  CDS (1)..(2928)
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctga

SEQ ID NO: 18 - protein, 976 aa, Homo sapiens [sequence omitted]

SEQ ID NO: 19 - DNA, 567 bp, Homo sapiens
  CDS (1)..(564)
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 20 - protein, 188 aa, Homo sapiens [sequence omitted]

SEQ ID NO: 21 - DNA, 1182 bp, Homo sapiens
  CDS (1)..(1179)
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 22 - protein, 393 aa, Homo sapiens [sequence omitted]

SEQ ID NO: 23 - DNA, 7671 bp, Homo sapiens
  CDS (1)..(7668)
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaatgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccgggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgccgctgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttctactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgccacgggggcacctgccaggaccgcgacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacatcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaatgagtgtgaatccaacccttgtgtcaacggcggcacctgcaaagacatgaccagtggctacgtgtgcacctgccgggagggcttcagcggtcccaactgccagaccaacatcaacgagtgtgcgtccaacccatgtctgaaccagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacgggctggcaagcagggcagacctgtgaggtcgacatcaacgagtgcgttctgagcccgtgccggcacggcgcatcctgccagaacacccacggcggctaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgatgtcaatgagtgcgactcacagccctgcctgcatggcggcacctgtcaggacggctgcggctcctacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggacggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctcaccccgtcccctgagtcccctgaccagtggtccagctcgtccccgcattccaacgtctccgactggtccgagggcgtctccagccctcccaccagcatgcagtcccagatcgcccgcattccggaggccttcaagtaa

SEQ ID NO: 24 - protein, 2556 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQAGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 25 - DNA, 570 bp, Homo sapiens
  CDS (1)..(567)
atgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaa

SEQ ID NO: 26 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 27 - DNA, 3207 bp, Homo sapiens
  CDS (1)..(3204)
atgcctccaagaccatcatcaggtgaactgtggggcatccacttgatgcccccaagaatcctagtggaatgtttactaccaaatggaatgatagtgactttagaatgcctccgtgaggctacattagtaactataaagcatgaactatttaaagaagcaagaaaataccctctccatcaacttcttcaagatgaatcttcttacattttcgtaagtgttacccaagaagcagaaagggaagaattttttgatgaaacaagacgactttgtgatcttcggctttttcaaccatttttaaaagtaattgaaccagtaggcaaccgtgaagaaaagatcctcaatcgagaaattggttttgctatcggcatgccagtgtgcgaatttgatatggttaaagatcctgaagtacaggacttccgaagaaatattcttaatgtttgtaaagaagctgtggatcttagggatcttaattcacctcatagtagagcaatgtatgtctatccgccacatgtagaatcttcaccagagctgccaaagcacatatataataaattggatagaggccaaataatagtggtgatttgggtaatagtttctccaaataatgacaagcagaagtatactctgaaaatcaaccatgactgtgtgccagaacaagtaattgctgaagcaatcaggaaaaaaactagaagtatgttgctatcatctgaacaattaaaactctgtgttttagaatatcagggcaagtacattttaaaagtgtgtggatgtgatgaatacttcctagaaaaatatcctctgagtcagtataagtatataagaagctgtataatgcttgggaggatgcccaatttgaagatgatggctaaagaaagcctttattctcaactgccaatggactgttttacaatgccatcttattccagacgcatttccacagctacaccatatatgaatggagaaacatctacaaaatccctttgggttataaatagagcactcagaataaaaattctttgtgcaacctacgtgaatctaaatattcgagacattgacaagatttatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaggaaggatgaaacacaaaaggtacagatgaagtttttagttgagcaaatgaggcgaccagatttcatggatgccctacagggcttgctgtctcctctaaaccctgctcatcaactaggaaacctcaggcttaaagagtgtcgaattatgtcttctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactga

SEQ ID NO: 28 - protein, 1068 aa, Homo sapiens
MPPRPSSGELWGIHLMPPRILVECLLPNGMIVTLECLREATLVTIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPHVESSPELPKHIYNKLDRGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLKMMAKESLYSQLPMDCFTMPSYSRRISTATPYMNGETSTKSLWVINRALRIKILCATYVNLNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQERKDETQKVQMKFLVEQMRRPDFMDALQGLLSPLNPAHQLGNLRLKECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 29 - DNA, 1212 bp, Homo sapiens
  CDS (1)..(1209)
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 30 - protein, 403 aa, Homo sapiens [sequence omitted]

SEQ ID NO: 31 - DNA, 1182 bp, Homo sapiens
  CDS (1)..(1179)
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 32 - protein, 393 aa, Homo sapiens [sequence omitted]

SEQ ID NO: 33 - DNA, 31 bp, Artificial Sequence
  Description of Artificial Sequence Synthetic primer
acgttggatgagccaatggttcagaaacaaa

SEQ ID NO: 34 - DNA, 31 bp, Artificial Sequence
  Description of Artificial Sequence Synthetic primer
acgttggatgtgacacaaagactggcttaca

SEQ ID NO: 35 - DNA, 30 bp, Artificial Sequence
  Description of Artificial Sequence Synthetic primer
acgttggatgagcagtgtcacagcacccta

SEQ ID NO: 36 - DNA, 30 bp, Artificial Sequence
  Description of Artificial Sequence Synthetic primer
acgttggatgctttgtgcctggctgattct

SEQ ID NO: 37 - DNA, 30 bp, Artificial Sequence
  Description of Artificial Sequence Synthetic primer
acgttggatgtcctcaaacagctcaaacca

SEQ ID NO: 38 - DNA, 30 bp, Artificial Sequence
  Description of Artificial Sequence Synthetic primer
acgttggatggcagcatttactgcagcttg

SEQ ID NO: 39 - DNA, 30 bp, Artificial Sequence
  Description of Artificial Sequence Synthetic primer
acgttggatgccaagagaaagaggcagaaa

SEQ ID NO: 40 - DNA, 30 bp, Artificial Sequence
  Description of Artificial Sequence Synthetic primer
acgttggatgtgttggcatggcagaaataa

SEQ ID NO: 41 - DNA, 32 bp, Artificial Sequence
  Description of Artificial Sequence Synthetic primer
acgttggatgtgcttgctctgataggaaaatg

SEQ ID NO: 42 - DNA, 29 bp, Artificial Sequence
  Description of Artificial Sequence Synthetic primer
acgttggatgctgatgggacccactccat

SEQ ID NO: 43 - DNA, 30 bp, Artificial Sequence
  Description of Artificial Sequence Synthetic primer
acgttggatgtcactggcagcaacagtctt

SEQ ID NO: 44 - DNA, 31 bp, Artificial Sequence
  Description of Artificial Sequence Synthetic primer
acgttggatgcaggattgcctttaccactca

SEQ ID NO: 45 - DNA, 30 bp, Artificial Sequence
  Description of Artificial Sequence Synthetic primer
acgttggatgccaaccaagctctcttgagg

SEQ ID NO: 46 - DNA, 30 bp, Artificial Sequence
  Description of Artificial Sequence Synthetic primer
acgttggatgccttatacaccgtgccgaac

SEQ ID NO: 47 - DNA, 30 bp, Artificial Sequence
  Description of Artificial Sequence Synthetic primer
acgttggatgtcgaggatttccttgttggc

SEQ ID NO: 48 - DNA, 30 bp, Artificial Sequence
  Description of Artificial Sequence Synthetic primer
acgttggatggatcccagaaggtgagaaag

SEQ ID NO: 49 - DNA, 30 bp, Artificial Sequence
  Description of Artificial Sequence Synthetic primer
acgttggatgtgttcccggacatagtccag

SEQ ID NO: 50 - DNA, 30 bp, Artificial Sequence
  Description of Artificial Sequence Synthetic primer
acgttggatgatctgcctcacctccaccgt

SEQ ID NO: 51 - DNA, 30 bp, Artificial Sequence
  Description of Artificial Sequence Synthetic primer
acgttggatgcctccttctgcatggtattc

SEQ ID NO: 52 - DNA, 30 bp, Artificial Sequence
  Description of Artificial Sequence Synthetic primer
acgttggatggcagcatgtcaagatcacag

SEQ ID NO: 53 - DNA, 30 bp, Artificial Sequence
  Description of Artificial Sequence Synthetic primer
acgttggatgcacgggaaagtggtgaagat

SEQ ID NO: 54 - DNA, 30 bp, Artificial Sequence
  Description of Artificial Sequence Synthetic primer
acgttggatgcattgcccctgacaacatag

SEQ ID NO: 55 - DNA, 31 bp, Artificial Sequence
  Description of Artificial Sequence Synthetic primer
acgttggatgagctttctcacaagcatttgg

SEQ ID NO: 56 - DNA, 31 bp, Artificial Sequence
  Description of Artificial Sequence Synthetic primer
acgttggatggctctgagaaaggcattagaa

SEQ ID NO: 57 - DNA, 30 bp, Artificial Sequence
  Description of Artificial Sequence Synthetic primer
acgttggatgtcatggtcggatcacaaaga

SEQ ID NO: 58 - DNA, 32 bp, Artificial Sequence
  Description of Artificial Sequence Synthetic primer
acgttggatggagaatgggtactcacgtttcc

SEQ ID NO: 59 - DNA, 37 bp, Artificial Sequence
  Description of Artificial Sequence Synthetic primer
acgttggatgtcattatttttattataaggcctgctg

SEQ ID NO: 60 - DNA, 31 bp, Artificial Sequence
  Description of Artificial Sequence Synthetic primer
acgttggatgagaatggtcctgcaccagtaa

SEQ ID NO: 61 - DNA, 29 bp, Artificial Sequence
  Description of Artificial Sequence Synthetic primer
acgttggatgggagcatgtacccgagagg

SEQ ID NO: 62 - DNA, 30 bp, Artificial Sequence
  Description of Artificial Sequence Synthetic primer
acgttggatggaagtggaaggagctgttgc

SEQ ID NO: 63 - DNA, 30 bp, Artificial Sequence
  Description of Artificial Sequence Synthetic primer
acgttggatgcaacagctccttccacttcc

SEQ ID NO: 64 - DNA, 28 bp, Artificial Sequence
  Description of Artificial Sequence Synthetic primer
acgttggatgatcatctgctggccgtgt

SEQ ID NO: 65 - DNA, 30 bp, Artificial Sequence
  Description of Artificial Sequence Synthetic primer
acgttggatgcaacaggttcttgctggtgt

SEQ ID NO: 66 - DNA, 31 bp, Artificial Sequence
  Description of Artificial Sequence Synthetic primer
acgttggatggagagacaggatcaggtcagc

SEQ ID NO: 67 - DNA, 30 bp, Artificial Sequence
  Description of Artificial Sequence Synthetic primer
acgttggatgtggtgaaacctgtttgttgg

SEQ ID NO: 68 - DNA, 34 bp, Artificial Sequence
  Description of Artificial Sequence Synthetic primer
acgttggatgcctttcagagaaaataatgctcct

SEQ ID NO: 69 - DNA, 30 bp, Artificial Sequence
  Description of Artificial Sequence Synthetic primer
acgttggatgcccctccatcaacttcttca

SEQ ID NO: 70 - DNA, 30 bp, Artificial Sequence
  Description of Artificial Sequence Synthetic primer
acgttggatgaaaagccgaaggtcacaaag

SEQ ID NO: 71 - DNA, 32 bp, Artificial Sequence
  Description of Artificial Sequence Synthetic primer
acgttggatggacaaagaacagctcaaagcaa

SEQ ID NO: 72 - DNA, 33 bp, Artificial Sequence
  Description of Artificial Sequence Synthetic primer
acgttggatgtttagcacttacctgtgactcca

SEQ ID NO: 73 - DNA, 30 bp, Artificial Sequence
  Description of Artificial Sequence Synthetic primer
acgttggatggagcaagaggctttggagta

SEQ ID NO: 74 - DNA, 32 bp, Artificial Sequence
  Description of Artificial Sequence Synthetic primer
acgttggatgatccaatccatttttgttgtcc

SEQ ID NO: 75 - DNA, 37 bp, Artificial Sequence
  Description of Artificial Sequence Synthetic primer
acgttggatgcttattctgaggttatctttttaccac

SEQ ID NO: 76 - DNA, 33 bp, Artificial Sequence
  Description of Artificial Sequence Synthetic primer
acgttggatgtgcacatatcattacaccagttc

SEQ ID NO: 77 - DNA, 30 bp, Artificial Sequence
  Description of Artificial Sequence Synthetic primer
acgttggatgttttctgtccaccagggagt

SEQ ID NO: 78 - DNA, 35 bp, Artificial Sequence
  Description of Artificial Sequence Synthetic primer
acgttggatgtccagatgattctttaacaggtagc

SEQ ID NO: 79 - DNA, 35 bp, Artificial Sequence
  Description of Artificial Sequence Synthetic primer
acgttggatgggtgaagatatattcctccaattca

SEQ ID NO: 80 - DNA, 36 bp, Artificial Sequence
  Description of Artificial Sequence Synthetic primer
acgttggatgttctcccaatgaaagtaaagtacaaa

SEQ ID NO: 81 - DNA, 30 bp, Artificial Sequence
  Description of Artificial Sequence Synthetic primer
acgttggatgcaagcagtcacagcacatga

SEQ ID NO: 82 - DNA, 30 bp, Artificial Sequence
  Description of Artificial Sequence Synthetic primer
acgttggatgctgctcaccatcgctatctg

SEQ ID NO: 83 - DNA, 30 bp, Artificial Sequence
  Description of Artificial Sequence Synthetic primer
acgttggatgtggctctgactgtaccacca

SEQ ID NO: 84 - DNA, 30 bp, Artificial Sequence
  Description of Artificial Sequence Synthetic primer
acgttggatgccagtgtgatgatggtgagg

SEQ ID NO: 85 - DNA, 30 bp, Artificial Sequence
  Description of Artificial Sequence Synthetic primer
acgttggatgctactgggacggaacagctt

SEQ ID NO: 86 - DNA, 30 bp, Artificial Sequence
  Description of Artificial Sequence Synthetic primer
acgttggatggcttcttgtcctgcttgctt

SEQ ID NO: 87 - DNA, 30 bp, Artificial Sequence
  Description of Artificial Sequence Synthetic primer
acgttggatgggcttgtgagtggatgggta

SEQ ID NO: 88 - DNA, 32 bp, Artificial Sequence
  Description of Artificial Sequence Synthetic primer
acgttggatggcaaaatcacattattgccaac

SEQ ID NO: 89 - DNA, 30 bp, Artificial Sequence
  Description of Artificial Sequence Synthetic primer
acgttggatgggtagagtgtgcgtggctct

SEQ ID NO: 90 - DNA, 30 bp, Artificial Sequence
  Description of Artificial Sequence Synthetic primer
acgttggatgaggtgccatcattcttgagg

SEQ ID NO: 91 - DNA, 32 bp, Artificial Sequence
  Description of Artificial Sequence Synthetic primer
acgttggatgtctgtttggcttgacttgactt

SEQ ID NO: 92 - DNA, 35 bp, Artificial Sequence
  Description of Artificial Sequence Synthetic primer
acgttggatgtcaccacattacatacttaccatgc

SEQ ID NO: 93 - DNA, 31 bp, Artificial Sequence
  Description of Artificial Sequence Synthetic primer
acgttggatggtttctcccttctcaggattc

SEQ ID NO: 94 - DNA, 35 bp, Artificial Sequence
  Description of Artificial Sequence Synthetic primer
acgttggatgcccacctataatggtgaatatcttc

SEQ ID NO: 95 - DNA, 30 bp, Artificial Sequence
  Description of Artificial Sequence Synthetic primer
acgttggatgattgacttgtgctccccact

SEQ ID NO: 96 - DNA, 30 bp, Artificial Sequence
  Description of Artificial Sequence Synthetic primer
acgttggatgccccagctcactgatcttct

SEQ ID NO: 97 - DNA, 83 bp, Artificial Sequence
  Description of Artificial Sequence Synthetic primer
actgactgactgactgactgactgactgactgactgactgactgactgactgactgactgatcccaatggttcagaaacaaat

SEQ ID NO: 98 - DNA, 83 bp, Artificial Sequence
  Description of Artificial Sequence Synthetic primer
tgactgactgactgactgactgactgactgactgactgactgactgactgactgactgactgatcaccaaatccagcagactg

SEQ ID NO: 99 - DNA, 76 bp, Artificial Sequence
  Description of Artificial Sequence Synthetic primer
gactgactgactgactgactgactgactgactgactgactgactgactgatcgtgctttatttttaggtacttctc

SEQ ID NO: 100 - DNA, 76 bp, Artificial Sequence
  Description of Artificial Sequence Synthetic primer
gactgactgactgactgactgactgactgactgactgactgactgactgactgatcagagaaagaggcagaaaaaa

SEQ ID NO: 101 - DNA, 54 bp, Artificial Sequence
  Description of Artificial Sequence Synthetic primer
tgactgactgactgactgactgactgactgactggtgattttggtctagctaca

SEQ ID NO: 102 - DNA, 45 bp, Artificial Sequence
  Description of Artificial Sequence Synthetic primer
gactgactgactgactgactgactgtgattttggtctagctacag

SEQ ID NO: 103 - DNA, 75 bp, Artificial Sequence
  Description of Artificial Sequence Synthetic primer
gactgactgactgactgactgactgactgactgactgactgactgactgactgactgcagcaacagtcttacctg

SEQ ID NO: 104 - DNA, 75 bp, Artificial Sequence
  Description of Artificial Sequence Synthetic primer
ctgactgactgactgactgactgactgactgactgactgactgactgactgactgaaccagaatggattccagag

SEQ ID NO: 105 - DNA, 25 bp, Artificial Sequence
  Description of Artificial Sequence Synthetic primer
gactggcaacagtcttacctggact

SEQ ID NO: 106 - DNA, 39 bp, Artificial Sequence
  Description of Artificial Sequence Synthetic primer
actgactgactgactgataacagtcttacctggactctg

SEQ ID NO: 107 - DNA, 45 bp, Artificial Sequence
  Description of Artificial Sequence Synthetic primer
ctgactgactgactgactgactgactgctggactctggaatccat

SEQ ID NO: 108 - DNA, 25 bp, Artificial Sequence
  Description of Artificial Sequence Synthetic primer
ctgactgtggactctggaatccatt

SEQ ID NO: 109 - DNA, 85 bp, Artificial Sequence
  Description of Artificial Sequence Synthetic primer
ctgactgactgactgactgactgactgactgactgactgactgactgactgactgactgactgactgacagagaaggagctgtgg

SEQ ID NO: 110 - DNA, 85 bp, Artificial Sequence
  Description of Artificial Sequence Synthetic primer
gactgactgactgactgactgactgactgactgactgactgactgactgactgactgactgactgacctcagagaaggagctgtg

SEQ ID NO: 111 - DNA, 55 bp, Artificial Sequence
  Description of Artificial Sequence Synthetic primer
gactgactgactgactgactgactgactgactgactgtgcctttaccactcagag

SEQ ID NO: 112 - DNA, 45 bp, Artificial Sequence
  Description of Artificial Sequence Synthetic primer
ctgactgactgactgactgactgactgttgcctttaccactcaga

SEQ ID NO: 113 - DNA, 45 bp, Artificial Sequence
  Description of Artificial Sequence Synthetic primer
gactgactgactgactgactgactgtcaaaaagatcaaagtgctg

SEQ ID NO: 114 - DNA, 35 bp, Artificial Sequence
  Description of Artificial Sequence Synthetic primer
ctgactgactgactgactgttcccgtcgctatcaa

SEQ ID NO: 115 - DNA, 17 bp, Artificial Sequence
  Description of Artificial Sequence Synthetic primer
tggctttcggagatgtt

SEQ ID NO: 116 - DNA, 35 bp, Artificial Sequence
  Description of Artificial Sequence Synthetic primer
ctgactgactgactgactgtcccgtcgctatcaag

SEQ ID NO: 117 - DNA, 35 bp, Artificial Sequence
  Description of Artificial Sequence Synthetic primer
gactgactgactgactgattggctttcggagatgt

SEQ ID NO: 118 - DNA, 45 bp, Artificial Sequence
  Description of Artificial Sequence Synthetic primer
ctgactgactgactgactgactgactgactaagggcatgagctgc

SEQ ID NO: 119 - DNA, 45 bp, Artificial Sequence
  Description of Artificial Sequence Synthetic primer
gactgactgactgactgactgactgacagatcacagattttgggc

SEQ ID NO: 120 - DNA, 69 bp, Artificial Sequence
  Description of Artificial Sequence Synthetic primer
gactgactgactgactgactgactgactgactgactgactgactgactgactactttggattggctcga

SEQ ID NO: 121 - DNA, 45 bp, Artificial Sequence
  Description of Artificial Sequence Synthetic primer
actgactgactgactgactgtttggttttaaattatggagtatgt

SEQ ID NO: 122 - DNA, 54 bp, Artificial Sequence
  Description of Artificial Sequence Synthetic primer
gactgactgactgactgactgactgactgactgacgattttggtctagccagag

SEQ ID NO: 123 - DNA, 25 bp, Artificial Sequence
  Description of Artificial Sequence Synthetic primer
gactgactgctcttgcctacgccac

SEQ ID NO: 124 - DNA, 25 bp, Artificial Sequence
  Description of Artificial Sequence Synthetic primer
ctgactcttgtggtagttggagctg

SEQ ID NO: 125 - DNA, 28 bp, Artificial Sequence
  Description of Artificial Sequence Synthetic primer
tgactgactgatggtagttggagctggt

SEQ ID NO: 126 - DNA, 28 bp, Artificial Sequence
  Description of Artificial Sequence Synthetic primer
gactgactgacggtagttggagctggtg

SEQ ID NO: 127 - DNA, 83 bp, Artificial Sequence
  Description of Artificial Sequence Synthetic primer
ctgactgactgactgactgactgactgactgactgactgactgactgactgactgactgactgactgacgcaccaccaccacc

SEQ ID NO: 128 - DNA, 90 bp, Artificial Sequence
  Description of Artificial Sequence Synthetic primer
actgactgactgactgactgactgactgactgactgactgactgactgactgactgactgactgactgactgactgactcagccgcgtgc

SEQ ID NO: 129 - DNA, 25 bp, Artificial Sequence
  Description of Artificial Sequence Synthetic primer
gactgactgcttttcccaacaccac

SEQ ID NO: 130 - DNA, 72 bp, Artificial Sequence
  Description of Artificial Sequence Synthetic primer
ctgactgactgactgactgactgactgactgactgactgactgactgactgactgagtggtggttggagcag

SEQ ID NO: 131 - DNA, 63 bp, Artificial Sequence
  Description of Artificial Sequence Synthetic primer
gactgactgactgactgactgactgactgactgactgactgactgactcgcttttcccaacac

SEQ ID NO: 132 - DNA, 35 bp, Artificial Sequence
  Description of Artificial Sequence Synthetic primer
actgactgactgactgactggcgcttttcccaaca

SEQ ID NO: 133 - DNA, 55 bp, Artificial Sequence
  Description of Artificial Sequence Synthetic primer
gactgactgactgactgactgactgactgactgacacatactggatacagctgga

SEQ ID NO: 134 - DNA, 55 bp, Artificial Sequence
  Description of Artificial Sequence Synthetic primer
ctgactgactgactgactgactgactgactgactgcatactggatacagctggac

SEQ ID NO: 135 - DNA, 69 bp, Artificial Sequence
  Description of Artificial Sequence Synthetic primer
gactgactgactgactgactgactgactgactgactgactgactgactgctcatggcactgtactcttc

SEQ ID NO: 136 - DNA, 62 bp, Artificial Sequence
  Description of Artificial Sequence Synthetic primer
ctgactgactgactgactgactgactgactgactgactaagaattttttgatgaaacaagac

SEQ ID NO: 137 - DNA, 63 bp, Artificial Sequence
  Description of Artificial Sequence Synthetic primer
tgactgactgactgactgactgactgactgactgactgactgacttctcctgctcagtgattt

SEQ ID NO: 138 - DNA, 65 bp, Artificial Sequence
  Description of Artificial Sequence Synthetic primer
gactgactgactgactgactgactgactgactgactgactgactgatcctctctctgaaatcact

SEQ ID NO: 139 - DNA, 69 bp, Artificial Sequence
  Description of Artificial Sequence Synthetic primer
actgactgactgactgactgactgactgactgactgactgactgactgcctctctctgaaatcactgag

SEQ ID NO: 140 - DNA, 69 bp, Artificial Sequence
  Description of Artificial Sequence Synthetic primer
actgactgactgactgactgactgactgactgactgactgactgactgctctctctgaaatcactgagc

SEQ ID NO: 141 - DNA, 76 bp, Artificial Sequence
  Description of Artificial Sequence Synthetic primer
tgactgactgactgactgactgactgactgactgactgactgactgactgactgactgacgtccagccaccatgat

SEQ ID NO: 142 - DNA, 15 bp, Artificial Sequence
  Description of Artificial Sequence Synthetic primer
gtccagccaccatga

SEQ ID NO: 143 - DNA, 62 bp, Artificial Sequence
  Description of Artificial Sequence Synthetic primer
actgactgactgactgactgactgactgactgactgactgactgattttgttgtccagccac

SEQ ID NO: 144 - DNA, 35 bp, Artificial Sequence
  Description of Artificial Sequence Synthetic primer
tgactgactgactgacttgtaaagctggaaaggga

SEQ ID NO: 145 - DNA, 45 bp, Artificial Sequence
  Description of Artificial Sequence Synthetic primer
actgactgactgactgactgactagtaactattcccagtcagagg

SEQ ID NO: 146 - DNA, 54 bp, Artificial Sequence
  Description of Artificial Sequence Synthetic primer
actgactgactgactgactgactgactgactgactgtgaacttgtcttcccgtc

SEQ ID NO: 147 - DNA, 62 bp, Artificial Sequence
  Description of Artificial Sequence Synthetic primer
gactgactgactgactgactgactgactgactgactgactaaacagaacaagatgctaaaaa

SEQ ID NO: 148 - DNA, 90 bp, Artificial Sequence
  Description of Artificial Sequence Synthetic primer
gactgactgactgactgactgactgactgactgactgactgactgactgactgactgactgactgactgactgaccggaggttgtgaggc

SEQ ID NO: 149 - DNA, 54 bp, Artificial Sequence
  Description of Artificial Sequence Synthetic primer
gactgactgactgactgactgactgactgactgactgactcctccggttcatgc

SEQ ID NO: 150 - DNA, 69 bp, Artificial Sequence
  Description of Artificial Sequence Synthetic primer
actgactgactgactgactgactgactgactgactgactgactgactgactgactggggcggcatgaac

SEQ ID NO: 151 - DNA, 54 bp, Artificial Sequence
  Description of Artificial Sequence Synthetic primer
ctgactgactgactgactgactgactgactgactgactgacggcggcatgaacc

SEQ ID NO: 152 - DNA, 69 bp, Artificial Sequence
  Description of Artificial Sequence Synthetic primer
ctgactgactgactgactgactgactgactgactgactgactgactgactgaggaacagctttgaggtg

SEQ ID NO: 153 - DNA, 90 bp, Artificial Sequence
  Description of Artificial Sequence Synthetic primer
actgactgactgactgactgactgactgactgactgactgactgactgactgactgactgactgactgactgagaacagctttgaggtgc

SEQ ID NO: 154 - DNA, 97 bp, Artificial Sequence
  Description of Artificial Sequence Synthetic primer
tgactgactgactgactgactgactgactgactgactgactgactgactgactgactgactgactgactgactgactgagtcctgcttgcttacctc

SEQ ID NO: 155 - DNA, 18 bp, Artificial Sequence
  Description of Artificial Sequence Synthetic primer
tgatccccataagcatga

SEQ ID NO: 156 - DNA, 62 bp, Artificial Sequence
  Description of Artificial Sequence Synthetic primer
gactgactggactgactgactgactgactggactgactgactgagatccccataagcatgac

SEQ ID NO: 157 - DNA, 90 bp, Artificial Sequence
  Description of Artificial Sequence Synthetic primer
ctgactgactgactgactgactgactgactgactgactgactgactgactgactgactgactgactgactgagccaggtcttgatgtact

SEQ ID NO: 158 - DNA, 83 bp, Artificial Sequence
  Description of Artificial Sequence Synthetic primer
gactgactgactgactgactgactgactgactgactgactgactgactgactgactgactgactggacaaagaattggatctg

SEQ ID NO: 159 - DNA, 90 bp, Artificial Sequence
  Description of Artificial Sequence Synthetic primer
tgactgactgactgactgactgactgactgactgactgactgactgactgactgactgactgactgactcactttcccttgtagactgtt

SEQ ID NO: 160 - DNA, 32 bp, Artificial Sequence
  Description of Artificial Sequence Synthetic primer
gactgactacagtaaaaataggtgattttggt

SEQ ID NO: 161 - DNA, 83 bp, Artificial Sequence
  Description of Artificial Sequence Synthetic primer
gactgactgactgactgactgactgactgactgactgactgactgactgactgactgactgactgactgcttccgcacccagc

SEQ ID NO: 162 - DNA, 62 bp, Artificial Sequence
  Description of Artificial Sequence Synthetic primer
actgactgactgactgactgactgactgactgactgactgaccaaaaagatcaaagtgctgg

SEQ ID NO: 163 - DNA, 18 bp, Artificial Sequence
  Description of Artificial Sequence Synthetic primer
attctcgacacagcaggt

SEQ ID NO: 164 - DNA, 62 bp, Artificial Sequence
  Description of Artificial Sequence Synthetic primer
actgactgactgactgactgactgactgactgactgactgactgatctcgacacagcaggtc

SEQ ID NO: 165 - DNA, 54 bp, Artificial Sequence
  Description of Artificial Sequence Synthetic primer
tgactgactgactgactgactgactgactgactgctcattgcactgtactcctc

SEQ ID NO: 166 - DNA, 76 bp, Artificial Sequence
  Description of Artificial Sequence Synthetic primer
tgactgactgactgactgactgactgactgactgactgactgactgactgactgactgactcccaccttctgcttc

SEQ ID NO: 167 - DNA, 75 bp, Artificial Sequence
  Description of Artificial Sequence Synthetic primer
ctgactgactgactgactgactgactgactgactgactgactgactgactgactgaccagttctcccaccttctg

SEQ ID NO: 168 - DNA, 81 bp, Artificial Sequence
  Description of Artificial Sequence Synthetic primer
actgactgactgactgactgactgactgactgactgactgactgactgactgactgactggctcactgatcttctcaaagt

SEQ ID NO: 169 - DNA, 35 bp, Artificial Sequence
  Description of Artificial Sequence Synthetic primer
gtacttctcacttggtttgagctgtttgagaaaaa

SEQ ID NO: 170 - DNA, 35 bp, Artificial Sequence
  Description of Artificial Sequence Synthetic primer
aatcaatagtttttttctgcctctttctctaaaaa

SEQ ID NO: 171 - DNA, 35 bp, Artificial Sequence
  Description of Artificial Sequence Synthetic primer
gagatttcattgtagctagaccaaaatcacaaaaa

SEQ ID NO: 172 - DNA, 35 bp, Artificial Sequence
  Description of Artificial Sequence Synthetic primer
attccagagttcaggtaagactgttgctgcaaaaa

SEQ ID NO: 173 - DNA, 35 bp, Artificial Sequence
  Description of Artificial Sequence Synthetic primer
attccagagtgcaggtaagactgttgctgcaaaaa

SEQ ID NO: 174 - DNA, 35 bp, Artificial Sequence
  Description of Artificial Sequence Synthetic primer
attccagagtacaggtaagactgttgctgcaaaaa

SEQ ID NO: 175 - DNA, 40 bp, Artificial Sequence
  Description of Artificial Sequence Synthetic primer
gcagcaacagtcttacctgggctctggaatccattctggt

SEQ ID NO: 176 - DNA, 35 bp, Artificial Sequence
  Description of Artificial Sequence Synthetic primer
atggattccacagtccaggtaagactgttgaaaaa

SEQ ID NO: 177 - DNA, 35 bp, Artificial Sequence
  Description of Artificial Sequence Synthetic primer
atggatttcagagtccaggtaagactgttgaaaaa

SEQ ID NO: 178 - DNA, 35 bp, Artificial Sequence
  Description of Artificial Sequence Synthetic primer
atggattacagagtccaggtaagactgttgaaaaa

SEQ ID NO: 179 - DNA, 35 bp, Artificial Sequence
  Description of Artificial Sequence Synthetic primer
gtggcaccagcatggattccagagtccaggaaaaa

SEQ ID NO: 180 - DNA, 35 bp, Artificial Sequence
  Description of Artificial Sequence Synthetic primer
gtggcaccataatggattccagagtccaggaaaaa

SEQ ID NO: 181 - DNA, 35 bp, Artificial Sequence
  Description of Artificial Sequence Synthetic primer
gtggcaccacaatggattccagagtccaggaaaaa

SEQ ID NO: 182 - DNA, 35 bp, Artificial Sequence
  Description of Artificial Sequence Synthetic primer
gtggcaccaaaatggattccagagtccaggaaaaa

SEQ ID NO: 183 - DNA, 40 bp, Artificial Sequence
  Description of Artificial Sequence Synthetic primer
aatccattctggtgccactatcacagctccttctctgagt

SEQ ID NO: 184 - DNA, 40 bp, Artificial Sequence
  Description of Artificial Sequence Synthetic primer
tgccactaccacagctcctcctctgagtggtaaaggcaat

SEQ ID NO: 185 - DNA, 35 bp, Artificial Sequence
  Description of Artificial Sequence Synthetic primer
gcaccggagcacagcactttgatctttttgaaaaa

SEQ ID NO: 186 - DNA, 35 bp, Artificial Sequence
  Description of Artificial Sequence Synthetic primer
ttcttgatgactctggctagaccaaaatcaaaaaa

SEQ ID NO: 187 - DNA, 35 bp, Artificial Sequence
  Description of Artificial Sequence Synthetic primer
cctacgccagcagctccaactaccacaagtaaaaa

SEQ ID NO: 188 - DNA, 35 bp, Artificial Sequence
  Description of Artificial Sequence Synthetic primer
tcttgcctacgcaaccagctccaactaccaaaaaa

SEQ ID NO: 189 - DNA, 40 bp, Artificial Sequence
  Description of Artificial Sequence Synthetic primer
gaggctggcggccggcacgccggtggtggtggtgctgatg

SEQ ID NO: 190 - DNA, 40 bp, Artificial Sequence
  Description of Artificial Sequence Synthetic primer
gaagaccacgttggtgtgcggcacgcggctgagctcccgc

SEQ ID NO: 191 - DNA, 40 bp, Artificial Sequence
  Description of Artificial Sequence Synthetic primer
actggtggtggttggagcaagtggtgttgggaaaagcgca

SEQ ID NO: 192 - DNA, 40 bp, Artificial Sequence
  Description of Artificial Sequence Synthetic primer
actggtggtggttggagcagctggtgttgggaaaagcgca

SEQ ID NO: 193 - DNA, 40 bp, Artificial Sequence
  Description of Artificial Sequence Synthetic primer
tgcgcttttcccaacaccagctgctccaaccaccaccagt

SEQ ID NO: 194 - DNA, 40 bp, Artificial Sequence
  Description of Artificial Sequence Synthetic primer
ggtggtggttggagcaggttgtgttgggaaaagcgcactg

SEQ ID NO: 195 - DNA, 40 bp, Artificial Sequence
  Description of Artificial Sequence Synthetic primer
ggtggtggttggagcaggtgatgttgggaaaagcgcactg

SEQ ID NO: 196 - DNA, 40 bp, Artificial Sequence
  Description of Artificial Sequence Synthetic primer
ggtggtggttggagcaggtgttgttgggaaaagcgcactg

SEQ ID NO: 197 - DNA, 35 bp, Artificial Sequence
  Description of Artificial Sequence Synthetic primer
tactcttctggtccagctgtatccagtatgaaaaa

SEQ ID NO: 198 - DNA, 35 bp, Artificial Sequence
  Description of Artificial Sequence Synthetic primer
tactcttctcgtccagctgtatccagtatgaaaaa

SEQ ID NO: 199 - DNA, 40 bp, Artificial Sequence
  Description of Artificial Sequence Synthetic primer
catactggatacagctggacacgaagagtacagtgccatg

SEQ ID NO: 200 - DNA, 40 bp, Artificial Sequence
  Description of Artificial Sequence Synthetic primer
catactggatacagctggacatgaagagtacagtgccatg

SEQ ID NO: 201 - DNA, 35 bp, Artificial Sequence
  Description of Artificial Sequence Synthetic primer
tctttctccttctcagtgatttcagagagaaaaaa

SEQ ID NO: 202 - DNA, 40 bp, Artificial Sequence
  Description of Artificial Sequence Synthetic primer
aaacaaatgaatgatgcacttcatggtggctggacaacaa

SEQ ID NO: 203 - DNA, 40 bp, Artificial Sequence
  Description of Artificial Sequence Synthetic primer
aatgaatgatgcacatcatagtggctggacaacaaaaatg

SEQ ID NO: 204 - DNA, 35 bp, Artificial Sequence
  Description of Artificial Sequence Synthetic primer
acaccagttcctccctttccagctttacagaaaaa

SEQ ID NO: 205 - DNA, 35 bp, Artificial Sequence
  Description of Artificial Sequence Synthetic primer
acaccagttcatccctttccagctttacagaaaaa

SEQ ID NO: 206 - DNA, 40 bp, Artificial Sequence
  Description of Artificial Sequence Synthetic primer
taacagttcctgcatgggcagcatgaaccggaggcccatc

SEQ ID NO: 207 - DNA, 35 bp, Artificial Sequence
  Description of Artificial Sequence Synthetic primer
gcacaaacacacacctcaaagctgttccgtaaaaa

SEQ ID NO: 208 - DNA, 42 bp, Artificial Sequence
  Description of Artificial Sequence Synthetic primer
tgtaaaacgacggccagtgagcactgatgataaacacctcaa

SEQ ID NO: 209 - DNA, 40 bp, Artificial Sequence
  Description of Artificial Sequence Synthetic primer
caggaaacagctatgaccataggctgatccacatgacgtt

SEQ ID NO: 210 - DNA, 22 bp, Artificial Sequence
  Description of Artificial Sequence Synthetic primer
gatttgatggagttggacatgg

SEQ ID NO: 211 - DNA, 21 bp, Artificial Sequence
  Description of Artificial Sequence Synthetic primer
tgttcttgagtgaaggactga

SEQ ID NO: 212 - DNA, 39 bp, Artificial Sequence
  Description of Artificial Sequence Synthetic primer
tgtaaaacgacggccagtctgaggtgacccttgtctctg

SEQ ID NO: 213 - DNA, 39 bp, Artificial Sequence
  Description of Artificial Sequence Synthetic primer
caggaaacagctatgacctacagcttgcaaggactctgg

SEQ ID NO: 214 - DNA, 39 bp, Artificial Sequence
  Description of Artificial Sequence Synthetic primer
tgtaaaacgacggccagtggtaacatccacccagatcac

SEQ ID NO: 215 - DNA, 39 bp, Artificial Sequence
  Description of Artificial Sequence Synthetic primer
caggaaacagctatgacctgagcagggtctagagcagag

SEQ ID NO: 216 - DNA, 37 bp, Artificial Sequence
  Description of Artificial Sequence Synthetic primer
tgtaaaacgacggccagtcgaagccacactgacgtgc

SEQ ID NO: 217 - DNA, 38 bp, Artificial Sequence
  Description of Artificial Sequence Synthetic primer
caggaaacagctatgaccctccttatctcccctccccg

SEQ ID NO: 218 - DNA, 39 bp, Artificial Sequence
  Description of Artificial Sequence Synthetic primer
tgtaaaacgacggccagttcttcccatgatgatctgtcc

SEQ ID NO: 219 - DNA, 38 bp, Artificial Sequence
  Description of Artificial Sequence Synthetic primer
caggaaacagctatgacccctggtgtcaggaaaatgct

SEQ ID NO: 220 - DNA, 26 bp, Artificial Sequence
  Description of Artificial Sequence Synthetic primer
gcagcaagtatgatgagcaagctttc

SEQ ID NO: 221 - DNA, 24 bp, Artificial Sequence
  Description of Artificial Sequence Synthetic primer
cagatgctctgagaaaggcattag

SEQ ID NO: 222 - DNA, 41 bp, Artificial Sequence
  Description of Artificial Sequence Synthetic primer
tgtaaaacgacggccagtcatacattcgaaagaccctagcc

SEQ ID NO: 223 - DNA, 40 bp, Artificial Sequence
  Description of Artificial Sequence Synthetic primer
caggaaacagctatgaccatggattgtgcaattcctatgc

SEQ ID NO: 224 - DNA, 20 bp, Artificial Sequence
  Description of Artificial Sequence Synthetic primer
cttgtgccctgactttcaac

SEQ ID NO: 225 - DNA, 19 bp, Artificial Sequence
  Description of Artificial Sequence Synthetic primer
accagccctgtcgtctctc

SEQ ID NO: 226 - DNA, 20 bp, Artificial Sequence
  Description of Artificial Sequence Synthetic primer
aggcctctgattcctcactg

SEQ ID NO: 227 - DNA, 20 bp, Artificial Sequence
  Description of Artificial Sequence Synthetic primer
actgacaaccacccttaacc

SEQ ID NO: 228 - DNA, 21 bp, Artificial Sequence
  Description of Artificial Sequence Synthetic primer
tcatcttgggcctgtgttatc

SEQ ID NO: 229 - DNA, 20 bp, Artificial Sequence
  Description of Artificial Sequence Synthetic primer
gaaatcggtaagaggtgggc

SEQ ID NO: 230 - DNA, 22 bp, Artificial Sequence
  Description of Artificial Sequence Synthetic primer
tttccttactgcctcttgcttc

SEQ ID NO: 231 - DNA, 24 bp, Artificial Sequence
  Description of Artificial Sequence Synthetic primer
ggaaaggtgataaaagtgaatctg

SEQ ID NO: 232 - DNA, 18 bp, Artificial Sequence
  Description of Artificial Sequence Synthetic primer
tgtaaaacgacggccagt

SEQ ID NO: 233 - DNA, 18 bp, Artificial Sequence
  Description of Artificial Sequence Synthetic primer
caggaaacagctatgacc

SEQ ID NO: 234 - DNA, 24 bp, Artificial Sequence
  Description of Artificial Sequence Synthetic primer
gcaccatctcacaattgccagtta

SEQ ID NO: 235 - DNA, 21 bp, Artificial Sequence
  Description of Artificial Sequence Synthetic primer
aaaaggtgggcctgaggttca

SEQ ID NO: 236 - DNA, 10 bp, Homo sapiens
acgttggatg

SEQ ID NO: 237 - DNA, 10 bp, Homo sapiens
ccaccagctc

SEQ ID NO: 238 - DNA, 10 bp, Homo sapiens
ccaycagctc

SEQ ID NO: 239 - DNA, 10 bp, Homo sapiens
tcatcacgca

SEQ ID NO: 240 - DNA, 10 bp, Homo sapiens
tcatcaygca

SEQ ID NO: 241 - DNA, 10 bp, Homo sapiens
gggctggcca

SEQ ID NO: 242 - DNA, 10 bp, Homo sapiens
gggckggcca

SEQ ID NO: 243 - DNA, 10 bp, Homo sapiens
gccaccagct

SEQ ID NO: 244 - DNA, 10 bp, Homo sapiens
gccamcagct

SEQ ID NO: 245 - DNA, 10 bp, Homo sapiens
tacagtgaaa

SEQ ID NO: 246 - DNA, 10 bp, Homo sapiens
tacagwgaaa

SEQ ID NO: 247 - DNA, 10 bp, Homo sapiens
gggctggcca

SEQ ID NO: 248 - DNA, 10 bp, Homo sapiens
gggckggcca